(19) United States
(12) Patent Application Publication
(10) Pub. No.: US 2024/0009290 A1
(43) Pub. Date: Jan. 11, 2024

(54) NOVEL VAR2CSA IMMUNOGENS AND METHODS OF USE THEREOF

(71) Applicant: The United States of America,as represented by the Secretary,Department of Health and Human Services, Bethesda, MD (US)

(72) Inventors: Niraj H. Tolia, Bethesda, MD (US); Rui Ma, Bethesda, MD (US); Patrick E. Duffy, Washington, DC (US); Jonathan P. Renn, Falls Church, VA (US)

(73) Assignee: The United States of America,as represented by the Secretary,Department of Health and Human Services, Bethesda, MD (US)

(21) Appl. No.: 18/037,837

(22) PCT Filed: Nov. 18, 2021
(86) PCT No.: PCT/US2021/059941

§ 371 Date: May 19, 2023

Related U.S. Application Data
(60) Provisional application No. 63/115,729, filed on Nov. 19, 2020.

(51) Int. Cl. A61K 39/015; C07K 14/445; A61P 33/06

(52) U.S. Cl. CPC A61K 39/015; C07K 14/445; A61P 33/06; A61K 2039/55566

(57) ABSTRACT

The disclosure provides immunogen polypeptides comprising fragments of VAR2CSA protein expressed by P. falciparum. Aspects of the disclosed immunogen polypeptides comprise all or portions of the CSA binding regions of VAR2CSA as identified by a structural study of VAR2CSA conducted by the inventors. Also provided are compositions comprising such immunogen polypeptides, and methods of using the immunogen polypeptides for vaccination and treatment of disease.

### CROSS-REFERENCE TO RELATED APPLICATION

This patent application claims the benefit of U.S. Provisional Patent Application No. 63/115,729 filed Nov. 19, 2020, which was filed with color figures, and which is incorporated by reference in its entirety herein.

### STATEMENT REGARDING FEDERALLY SPONSORED RESEARCH OR DEVELOPMENT

This invention was made with Government support under project numbers ZIAAI001237 and ZIAAI001237 by the National Institutes of Health, National Institute of Allergy and Infectious Diseases. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,085,939 Byte ASCII (Text) file named “758113_ST25,” created on Nov. 18, 2021.

### BACKGROUND OF THE INVENTION

The ability to sequester in different organs, combined with sophisticated antigenic diversity, has made P. falciparum the deadliest malaria species that infects humans. Malaria during pregnancy is a major problem in sub-Saharan Africa, affecting an estimated 150 million pregnant women annually. Women can become susceptible to malaria infection during pregnancy despite the immunity that might have developed from prior P. falciparum infections. Pregnant women may also serve as a reservoir for parasites, which poses challenges to malaria eradication.

Placental malaria is caused by the accumulation of P. falciparum-infected erythrocytes in the placenta of pregnant women, resulting in high rates of maternal anemia, low birth weight, stillbirth and spontaneous pregnancy loss. Each year, up to 200,000 infant deaths and 10,000 maternal deaths are attributed to malaria infection in pregnancy globally. However, women naturally acquire resistance to placental malaria over successive pregnancies, providing a strong basis for the development of vaccines to prevent placental malaria.

P. falciparum expresses a family of proteins, referred to as erythrocyte membrane protein 1 (PfEMP1) that are translocated to the surface of the infected erythrocyte to enable adherence to different host organs and to evade the host immune response. VAR2CSA is a member of the PfEMP1 family that specifically binds to the syncytiotrophoblast surface receptor chondroitin sulfate A (CSA) leading to placental malaria. Additionally, diverse cancer cells express and present the form of chondroitin sulfate that is typically found in the placenta.

Due to its large size production of VAR2CSA protein for vaccine development and scientific study has proven challenging. Furthermore, the highly polymorphic nature of the extracellular domain of VAR2CSA in parasite isolates may hinder the development of a strain-transcending vaccine and effective therapeutics.

Despite the importance of VAR2CSA in both malaria and cancer, critical information has been lacking about the specific recognition mechanism for VAR2CSA binding CSA. Accordingly, improved vaccines and therapeutics based on a more complete picture of VAR2CSA structure and function are needed.

### BRIEF SUMMARY OF THE INVENTION

Aspects of the present disclosure provide an immunogen polypeptide, wherein the immunogen polypeptide comprises, consists essentially of, or consists of:
a) all or portions of the major CSA binding channels of VAR2CSA;
b) all or portions of the minor CSA binding channels of VAR2CSA;
c) all or portions of the arm segment of VAR2CSA; or
d) combinations thereof.

In other aspects, the present disclosure provides a pharmaceutical composition comprising the immunogen polypeptide, and a pharmaceutically acceptable carrier.

In other aspects, the present disclosure provides a pharmaceutical composition comprising a nucleic acid comprising a region encoding the immunogen polypeptide.

In other aspects, the present disclosure provides compositions for use in vaccinating a subject against malaria, wherein the composition comprises the disclosed immunogen polypeptides or nucleic acids encoding the disclosed immunogen polypeptides.

In other aspects, the disclosure provides a method of vaccinating a subject against malaria, the method comprising:
a) obtaining an immunogen polypeptide, wherein the immunogen polypeptide comprises, consists essentially of, or consists of:
   1) all or portions of the major CSA binding channels of VAR2CSA;
   2) all or portions of the minor CSA binding channels of VAR2CSA;
   3) all or portions of the arm of VAR2CSA; or
   4) combinations thereof;
b) preparing a pharmaceutical composition comprising an effective amount of the immunogen polypeptide and a pharmaceutically acceptable carrier; and
c) administering the pharmaceutical composition to the subject.

In other aspects, the present disclosure provides compositions for use in treating or preventing cancer in a subject, wherein the composition comprises the disclosed immunogen polypeptides or nucleic acids encoding the disclosed immunogen polypeptides.

In other aspects, the disclosure provides a method of treating or preventing cancer in a subject, the method comprising:
a) obtaining an immunogen polypeptide, wherein the immunogen polypeptide comprises, consists essentially of, or consists of:
   1) all or portions of the major CSA binding channels of VAR2CSA;
   2) all or portions of the minor CSA binding channels of VAR2CSA;
   3) all or portions of the arm of VAR2CSA; or
   4) combinations thereof;
b) preparing a pharmaceutical composition comprising an effective amount of the immunogen polypeptide and a pharmaceutically acceptable carrier; and
c) administering the pharmaceutical composition to the subject.

In another aspect, the disclosure provides a nucleic acid, optionally in a vector, encoding an immunogen polypeptide, wherein the immunogen polypeptide comprises, consists essentially of, or consists of:
a) all or portions of the major CSA binding channels of VAR2CSA;
b) all or portions of the minor CSA binding channels of VAR2CSA;
c) all or portions of the arm segment of VAR2CSA; or
d) combinations thereof.

In another aspect, the disclosure provides a method for producing an immunogen polypeptide in a cell in a subject, comprising administering to the subject a pharmaceutical composition comprising a modified messenger RNA (mmRNA) such that the mmRNA is introduced into the cell, wherein the mmRNA comprises a translatable region encoding the immunogen polypeptide, and wherein the immunogen polypeptide is produced in the cell. Additional aspects of the disclosure are as described herein.

### BRIEF DESCRIPTION OF THE DRAWINGS

FIG. 1A depicts a schematic of VAR2CSA NF54 primary structure colored by domain. Domains that were excluded from the ectodomain expression construct or could not be visualized in the final map are colored white. TM: transmembrane domain. ATS: Acidic terminal sequence. The alignments of PRIMVAC, PAMVAC and rVAR2 polypeptide are indicated below.

FIG. 1B depicts left two views of the Cryo-EM density for the 3.36 Å core structure. Right: the same two views of the atomic model corresponding to the map. Each domain is colored as in a. The CSA major and minor binding channel are highlighted by arrows.

FIG. 1C depicts two views of the Cryo-EM density for the entire complex with the model docked inside. The full-length density is the combination of the core and arm after local refinement.

FIG. 1D depicts a schematic drawing of the CSA-VAR2CSA NF54 complex. Each line indicates interactions between the connecting domains. The major binding channel and minor binding channel are highlighted by the hexagon and triangle.

FIG. 2A depicts the models of the NTS, six DBL domains and 2 ID regions are shown according to the order of the protein sequence. Each domain is colored according to FIG. 1a.

FIG. 2B depicts NTS unites DBL1X and DBL4ε. NTS is shown in surface while DBL1X and DBL4ε are shown in ribbon. All the domains are colored according to FIG. 1a.

FIG. 2C depicts a sequence alignment of NTS among different VAR2CSA variants. The range of the final model of the NTS is highlighted by the bar above the sequences. The alignment provides a comparison of the indicated amino acids from wild-type VAR2CSA variants from various strains (SEQ ID NOS: 1, 2 and 55-66).

FIG. 2D depicts ID3 is an a helix that connects DBL4ε and DBL5ε. ID3 is shown in surface while the rest of the molecule is shown in ribbon.

FIG. 2E depicts the models of VAR2CSA NF54 are colored in grey shown in two different views. The disulfides bonds are shown as dark circles.

FIG. 3A depicts two views of the structure showing a dodecamer of CSA is bound in the major binding channel. The Cryo-EM map density of NTS, DBL1X, DBL2X and DBL4≥ are shown in solid with transparency. The Cryo-EM density of CSA is shown in mesh overlaid on the CSA model in stick. The left and right monosaccharide are BDP-12 and BDP-2 indicated by the numbers 12 and 2, respectively. Density for the first monosaccharide of the chain is also observed and labeled with 1. Binding site 1 and 2 are highlighted by the dashed oval and rectangle respectively.

FIG. 3B depicts an electrostatic surface of VAR2CSA showing the positive charged binding channel of CSA.

FIG. 3C depicts detailed interactions between CSA and binding site 1. Each monosaccharide is numbered. The protein sequence number and side chains of the residues involved in CSA recognition are shown.

FIG. 3D depicts detailed interactions between CSA and binding site 1. Each monosaccharide is numbered. The protein sequence number and side chains of the residues involved in CSA recognition are shown.

FIG. 3E depicts detailed interactions between BDP-2 to ASG-5 and binding site 2. Each monosaccharide is numbered. The protein sequence number and side chains of the residues involved in the CSA recognition are shown.

FIG. 3F depicts the CSA molecule in the major binding channel is positioned as in FIG. 2A with numbering of each of the monosaccharide. The domains that each monosaccharide interact are indicated below.

FIG. 3G depicts partial sequence alignment of the residues involved in binding CSA in the major binding channel, the residues in major binding sites 1 and 2 are indicated by light and dark circles respectively. The surface exposed binding site on DBL2 is indicated by the dark line above the table. The alignment provides a comparison of the indicated amino acids from wild-type VAR2CSA variants from various strains (SEQ ID NOS: 1, 2 and 55-66).

FIG. 4A depicts domain boundaries of VAR2CSA NF54 and VAR2CSA FCR3 ectodomains. The protein sequence identity between the two is labeled.

FIG. 4B depicts two views of the Cryo-EM density for the 3.38 Å core region of VAR2CSA FCR3.

FIG. 4C depicts structural alignment of apo VAR2CSA FCR3, cross-linked VAR2CSA FCR3 and the CSA-VAR2CSA NF54 complex.

FIG. 4D depicts the Electrostatic surface of VAR2CSA FCR3 is shown on the left with a zoom-in view of the CSA binding sites on the right. The major and minor binding channel are indicated by arrows.

FIG. 5A depicts fourteen sequences of VAR2CSA that represent the diversity were analyzed using ConSurf Surface residues on a space filled model are shaded according to degree of conservation. The color key is shown below. Four different views are illustrated.

FIG. 5B depicts left: the atomic model of CSA-VAR2CSA NF54 complex. Right: space filling models of the CSA-VAR2CSA binding interface. Surface residues are shaded according to degree of conservation. The color key is shown below. The surface exposed major binding site 1 is highlighted by a black dotted circle.

FIG. 5C depicts left: the structural model of sequences comprising PRIMVAC and are shown in ribbon. The remainder of the VAR2CSA protein is shown in surface. Right: based on the variability analysis in a, PRIMVAC is shown in bold while the rest of the VAR2CSA molecule is shown in transparent.

FIG. 5D depicts left: the structural model of sequences comprising PAMVAC and are shown in ribbon. The remainder of the VAR2CSA protein is shown in surface. Right: based on the variability analysis in a, PAMVAC are shown in bold while the rest of the VAR2CSA molecule is shown in transparent.

FIG. 6 depicts human antibodies epitopes mapped on VAR2CSA. VAR2CSA structure is shown in surface. The characterized neutralizing epitopes are colored as illustrated: P57, P54 and P23-P25 are epitopes on DBL4ε. PAM8.1 epitope is a flexible loop on DBL3X that is missing the final structure and is colored by pink and illustrated by dash line. P62 on DBL3X and P63 on DBL5ε are shown. The cryptic epitopes P20 and P23 on DBL5ε are also shown.

FIG. 7A depicts domain boundaries of VAR2CSA NF54 and VAR2CSA FCR3 ectodomains that were used in the structural analysis.

FIG. 7B depicts Top: Size Exclusion Chromatography (SEC) profile of the VAR2CSA NF54 and VAR2CSA FCR3 proteins. Bottom: SDS PAGE analysis of the corresponding SEC fractions of VAR2CSA NF54 (left) and VAR2CSA FCR3 (right).

FIG. 8A depicts, a flow chart showing the image-processing pipeline for the cryo-EM data of VAR2CSA starting with 6,196 dose-fractionated movies collected on a 300-keV Titan Krios (FEI) equipped with a K2 Summit direct electron detector (Gatan). Data were processed in cryoSPARC. Full frame motion correction was done by cryoSPARC's own implementation. CTF estimation for each micrograph was calculated with Gctf. Particles were autopicked from each micrograph with the blob picker from cryoSPARC and then sorted by 2D classification for two rounds. The twelve highest-populated classes with clear features from the 2D classification are shown. The dataset contained 858,299 particles. A subset of particles was used to generate an ab initio map in cryoSPARC. Particles were classified into 5 classes using the low-pass-filtered (30 Å) ab initio map as a template. Class 1 was selected with 157,702 particles to conduct NU-refinement and generated a 3.87 Å map. A mask covering DBL5ε and DBL6ε domains were then used to perform local refinement and generated a 4.88 Å map. Class 1 and Class 4 which have a clear core density were selected again with 299,571 particles to conduct NU-refinement and generated a 3.5 Å map, local refinement improved the resolution of the core to 3.36 Å.

FIG. 8B depicts gold-standard FSC curves. The dotted line represents the 0.143 FSC cut-off.

FIG. 8C depicts angular distribution calculated in cryoSPARC for particle projections of the full-length protein (right) and the core (left). Heat map shows number of particles for each viewing angle.

FIG. 8D depicts local resolution of the core in two views. The representation of colors for different resolution are shown on the right.

FIG. 8E depicts local resolution of full length VAR2CSA in two views. The representation of colors for different resolution are shown on the right.

FIG. 8F depicts local resolution of the arm region in two views. The representation of colors for different resolution are shown on the right.

FIG. 8G depicts FSC calculated between the refined structures and the full map.

FIG. 8H depicts representative cryo-EM densities from the core machinery map.

FIG. 8I depicts representative cryo-EM densities from the arm with DBL5ε and DBL6ε model docked in.

FIG. 9A depicts a structural alignment of DBL1X with VarO_DBL1α1 (PDB: 2YK0, RMSD:3.18) and IT4var13 DBLβ (PDB:6s8t, RMSD: 2.94).

FIG. 9B depicts a structural alignment of DBL2X with varO_DBL1α1 (PDB: 2YKO, RMSD:5.75) and PF11_0521 DBLβ (PDB: 5mza, RMSD:4.85).

FIG. 9C depicts a structural alignment of DBL5ε with IT4var13 DBLβ (PDB: 6s8t, RMSD:8.37) and EBA-175 F2 domain (PDB: 1ZRO, RMSD:4.24).

FIG. 9D depicts DBL3X-4ε DBL domains are colored according to FIG. 1a.

FIG. 9E depicts DBL 5ε-6ε, DBL domains are colored according to FIG. 1a.

FIG. 9F depicts the crystal structure of EBA-175 (PDB: 1ZRO). The F1 and F2 domain are colored in light and dark grey respectively.

FIG. 9G depicts the crystal structure of EBA-140 (PDB: 4JNO).

FIG. 9H depicts a structural comparison of VAR2CSA DBL2X-ID and PfEMP1-VarO DBL1α-CIDR. Upper: atomic model of VAR2CSA DBL2X-ID2; Lower: Crystal structure of varO_DBL1α1-CIDRγ.

FIG. 9I depicts a structural alignment of VAR2CSA_ID2 with varO_CIDRγ subdomain2, RMSD=4.

FIG. 9J depicts a sequence alignment of the ID2 portion of wild-type VAR2CSA from P. falciparum strain NF54 (SEQ ID NO: 1) and varO_CIDRγ (SEQ ID NO 67).

FIG. 10A depicts one ASG monosaccharide could be built in a weak density found in the minor binding channel sandwiched by DBL2X and ID2a. The density is shown in mesh. The residues that involve in forming hydrogen bonds with the ASG monosaccharide are illustrated.

FIG. 10B depicts electrostatic surface of the proteins showing both major binding channel and minor binding channel are positively charged.

FIG. 10C depicts a partial sequence alignment of the residues involved in the minor binding channel. The residues that interact with the monosaccharide from DBL2X and ID2a are indicated by dark and light circles respectively. The alignment provides a comparison of the indicated amino acids from wild-type VAR2CSA variants from various strains (SEQ ID NOS: 1, 2 and 55-66).

FIG. 11A depicts flow chart showing the image-processing pipeline for the cryo-EM data of VAR2CSA starting with 100,108 dose-fractionated movies collected on a 300-keV Titan Krios (FEI) equipped with a K2 Summit direct electron detector (Gatan). Processing was done within cryoSPARC. Full frame motion correction was done by cryoSPARC's own implementation. CTF estimation for each micrograph was calculated with Gctf. Particles were autopicked from each micrograph with the blob picker from cryoSPARC and then sorted by 2D classification for two rounds to exclude bad particles. The twelve highest-populated classes with clear features from the 2D classification are shown. The dataset contained 783,088 particles. A subset of particles was used to generate an ab initio map in cryoSPARC. Particles were classified into 10 classes using the low-pass-filtered (30 Å) ab initio map as a template. Class 4 with a total of 271,442 particles was selected to conduct NU-refinement and generated a 4 Å map. A mask covering the arm region were then used to perform local refinement and generated a 4.69 Å map. The angular distribution calculated in cryoSPARC for particle projections are shown in heat map which shows number of particles for each viewing angle.

FIG. 11B depicts gold-standard FSC curves. The dotted line represents the 0.143 FSC cut-off, which indicates a nominal resolution of 4 Å (dashed) and 4.69 Å (solid) for the full length protein and arm region respectively.

FIG. 11C depicts local resolution of the full length VAR2CSA map in two views. The representation of colors for different resolution are shown on the right.

FIG. 11D depicts local resolution of the arm map in two views. The representation of colors for different resolution are shown on the right.

FIG. 11E depicts flow chart showing the image-processing pipeline for the cryo-EM data of cross-linked VAR2CSA starting with 4,739 dose-fractionated movies collected on a 300-keV Titan Krios (FEI) equipped with a K2 Summit direct electron detector (Gatan). All processing was done within cryoSPARC. Full frame motion correction was done by cryoSPARC's own implementation and a sample. CTF estimation for each micrograph was calculated with Gctf. Particles were auto picked from each micrograph with the blob picker from cryoSPARC and then sorted by 2D classification for two rounds to exclude bad particles. The twelve highest-populated classes with clear features from the 2D classification are shown. The dataset contained 505,409 particles. A subset of particles was used to generate an ab initio map in cryoSPARC. Particles were classified into 3 classes using the low-pass-filtered (30 Å) ab initio map as a template. Class 3 was selected to conduct NU-refinement and generated a 3.52 Å map. A mask covering the core was then used to perform local refinement and generated a 3.38 Å map. The angular distribution calculated in cryoSPARC for particle projections are shown in heat map which shows number of particles for each viewing angle.

FIG. 11F depicts gold-standard FSC curves. The dotted line represents the 0.143 FSC cut-off, which indicates a nominal resolution of 3.38 Å of the core.

FIG. 11G depicts local resolution of the cross-linked VAR2CSA FCR3 core map in two views. The representation of colors for different resolution are shown on the right.

FIG. 12A depicts an atomic model of the core of the cross-linked VAR2CSA FCR3.

FIG. 12B depicts FSC calculated between the refined structure and the full map.

FIG. 12C depicts a Representative cryo-EM densities from the core.

FIG. 12D depicts an Atomic model of full length VAR2CSA FCR3 docked in the 4.06 Å map.

FIG. 12E depicts FSC calculated between the refined core structure and the full map.

FIG. 12F depicts a representative cryo-EM densities from the core.

FIG. 12G depicts a representative cryo-EM densities from the arm with DBL5ε and DBL6ε model docked in.

FIG. 13A depicts the mechanism of placental sequestration of Plasmodium falciparum. In the placenta, the parasite expresses VAR2CSA on the surface of the infected erythrocytes. VAR2CSA specifically binds to the CSA on the placental synctiotrophoblast through two channels in its core domain with high affinity, leading to the sequestration of the parasite in the placenta and threatening the health of mother and baby.

FIG. 13B illustrates that cancer cells of various types harbor the same type of CSA on their surfaces. Conjugated VAR2CSA immunogen peptides can therefore be used to deliver drugs or labels specifically to tumor cells.

FIG. 14 depicts the structural model of rVAR2, which is shown in ribbon. The remainder of the VAR2CSA protein is shown in surface.

FIG. 15 depicts the results of electrophoresis for the NF54 Core ΔDBL3 (“major and minor channels”, SEQ ID NO: 3), NF54 Core (SEQ ID NO: 5), NF54 DBL56 (“arm”, SEQ ID NO: 7), and FCR3 DBL56 (“arm”, SEQ ID NO: 8).

FIGS. 16A and 16B depict domain structures of disclosed immunogen polypeptides. Each immunogen polypeptide is illustrated as a schematic representation pair which either highlight the deleted domains (FIG. 17A) or the final construct (FIG. 17B). The “min” refers to constructs that are designed by deleting ID1 and some long flexible loops.

FIG. 16C depicts schematic diagrams of the structures of disclosed immunogen polypeptides.

FIG. 17 is a photograph depicting SDS PAGE analysis of the VAR2CSA NF54 immunogens. The purified yield (mg/l) of each immunogen are indicated below the gel.

FIG. 18 is a graph depicting data demonstrating immunogen polypeptides retain CSA binding affinity. CSA was coated on ELISA plates. After blocking, 10 ug/ml immunogens were added into the wells and detected by HRP conjugated anti-His antibody after washing. MSP1 was used as a negative control.

FIG. 19 is a graph depicting antibody titers from rats after three immunizations with recombinant immunogen polypeptides adjuvated with CFA/IFA. The rat serum was collected on day 63 after three immunizations (administered on days 0, 21 and 42). Serum pooled from ten rats were used to generate the standard curve. The antibody titers of all disclosed immunogens show statistically significant differences from the control.

FIG. 20A and 20B are graphs depicting data demonstrating the disclosed immunogen polypeptides induce homologous inhibitory antibodies. Binding inhibition assay using purified IgGs (FIG. 21A: 1 mg/ml; FIG. 21B: 4 mg/ml) from pooled rat serum of each group after three vaccinations adjuvated with CFA/IFA. The 50% inhibition level used as a cutoff for inhibitory or non-inhibitory activity is highlighted as a dash line.

FIG. 21 is a graph depicting antibody titers from rats after three immunizations with recombinant immunogens adjuvated with AddaS03. The rat serum was collected on day 63 after three immunizations (administered on days 0, 21 and 42). A pool serum of 10 rats were used to generate the standard curve. The antibody titers of all disclosed immunogen polypeptides show statistically significant differences from the control.

FIG. 22A and 22B are graphs depicting data demonstrating that three specific immunogen polypeptides induce homologous inhibitory antibodies with AddaS03 adjuvant. Binding inhibition assay using IgGs (FIG. 23A: 1 mg/ml; FIG. 23B: 4 mg/ml) purified from the rat's pooled serum from each group after three vaccinations with the CFA/IFA adjuvants. The 50% inhibition level used as a cutoff for inhibitory or non-inhibitory activity is highlighted as a dash line.

FIG. 23 is a graph depicting a statistical comparison of the two adjuvants CFA/IFA and AddaS03 showing no difference between the groups. The p values are indicated above the table.

FIG. 24 is a graph depicting a statistical comparison of the antibody titers between the second (day 35) and third (day 63) immunization.

FIG. 25 is a graph depicting data demonstrating that three specific immunogen polypeptides formulated with AddaS03 induce strong homologous inhibitory antibodies upon either two or three vaccinations. The 50% inhibition level used as a cutoff for inhibitory or non-inhibitory activity is indicated as a dashed line.

Color versions of many of the figures described above were filed with Provisional Patent Application No. 63/115,729, filed Nov. 19, 2020, which is incorporated by reference in its entirety.

### DETAILED DESCRIPTION OF THE INVENTION

### Immunogen Polypeptides

As used herein, VAR2CSA refers to VAR2CSA proteins expressed by Plasmodium falciparum and variants of VAR2CSA expressed by Plasmodium falciparum. VAR2CSA is a member of the PfEMP1 family that specifically binds to the syncytiotrophoblast surface receptor chondroitin sulfate A (CSA). One variant of VAR2CSA according to the present disclosure is the wild-type VAR2CSA protein from P. falciparum strain NF54 (SEQ ID NO: 1). Another variant according to the present disclosure is the VAR2SCA protein from P. falciparum strain FCR3 (SEQ ID NO: 2). Another variant according to the present disclosure is the wild-type VAR2SCA protein from P. falciparum strain 7G8 (SEQ ID NO: 55). Another variant according to the present disclosure is the wild-type VAR2SCA protein from P. falciparum strain HB3 (SEQ ID NO: 56). Another variant according to the present disclosure is the wild-type VAR2SCA protein from P. falciparum strain CD01 (SEQ ID NO: 57). Another variant according to the present disclosure is the wild-type VAR2SCA protein from P. falciparum strain Dd2 (SEQ ID NO: 58). Another variant according to the present disclosure is the wild-type VAR2SCA protein from P. falciparum strain GA01 (SEQ ID NO: 59). Another variant according to the present disclosure is the wild-type VAR2SCA protein from P. falciparum strain GB4 (SEQ ID NO: 60). Another variant according to the present disclosure is the wild-type VAR2SCA protein from P. falciparum strain KE01 (SEQ ID NO: 61). Another variant according to the present disclosure is the wild-type VAR2SCA protein from P. falciparum strain KH01 (SEQ ID NO: 62). Another variant according to the present disclosure is the wild-type VAR2SCA protein from P. falciparum strain ML01 (SEQ ID NO: 63). Another variant according to the present disclosure is the wild-type VAR2SCA protein from P. falciparum strain SNO1 (SEQ ID NO: 64). Another variant according to the present disclosure is the wild-type VAR2SCA protein from P. falciparum strain TG01 (SEQ ID NO: 65). Another variant according to the present disclosure is the wild-type VAR2SCA protein from P. falciparum strain GN01 (SEQ ID NO: 66).

As described in detail in Example 1, the inventors have elucidated the cryo-EM structures of VAR2CSA from P. falciparum strain NF54 in complex with CSA and VAR2CSA from a second P. falciparum strain FCR3.

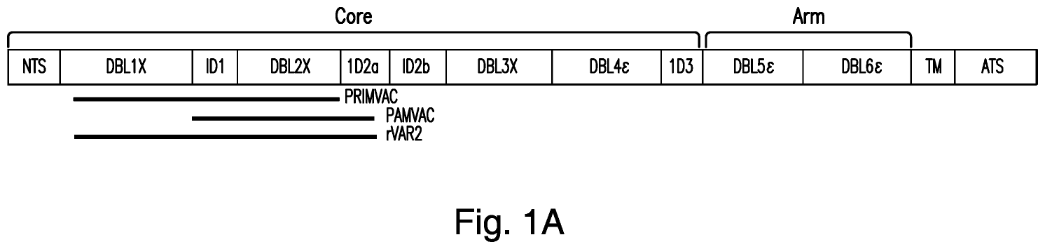

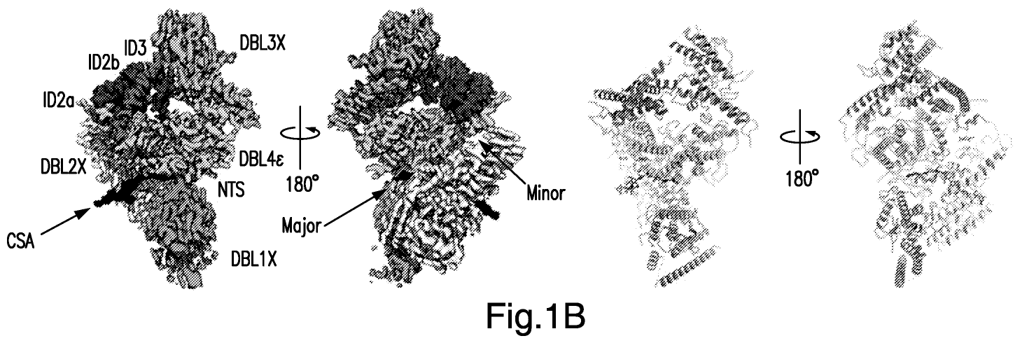

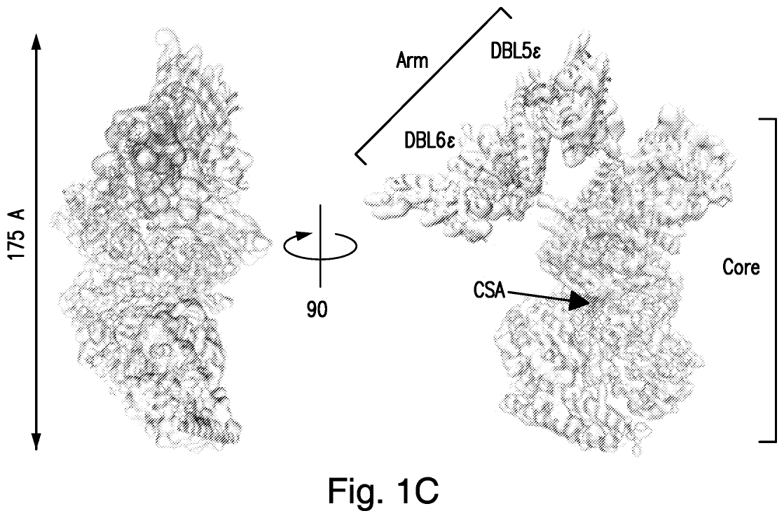

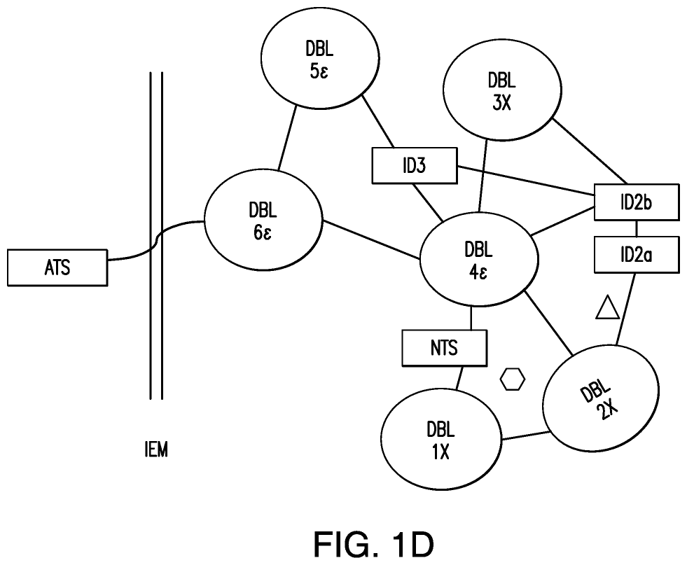

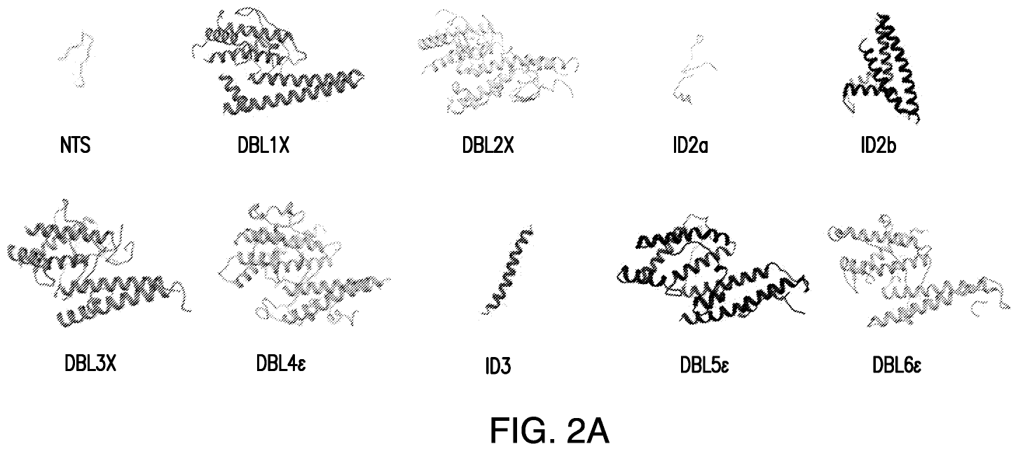

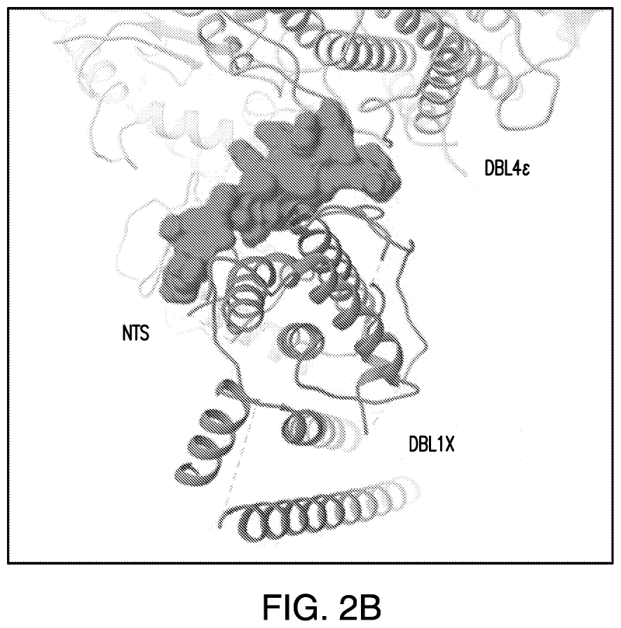

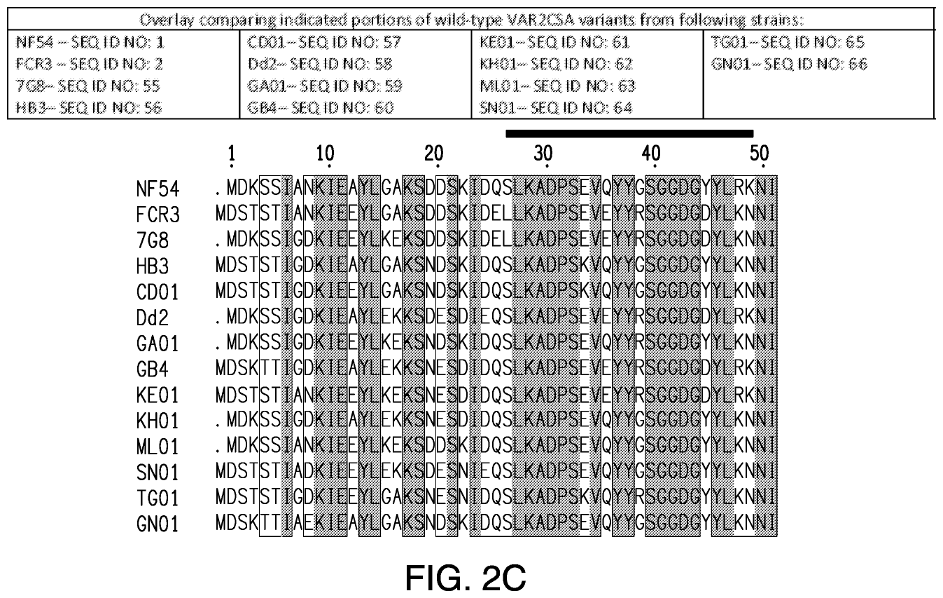

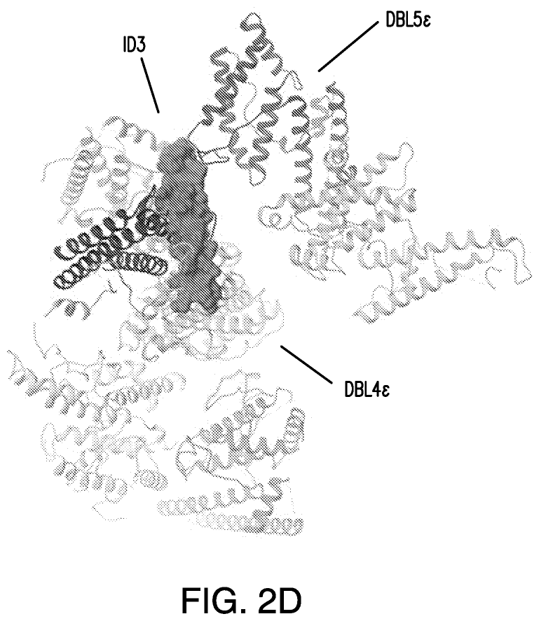

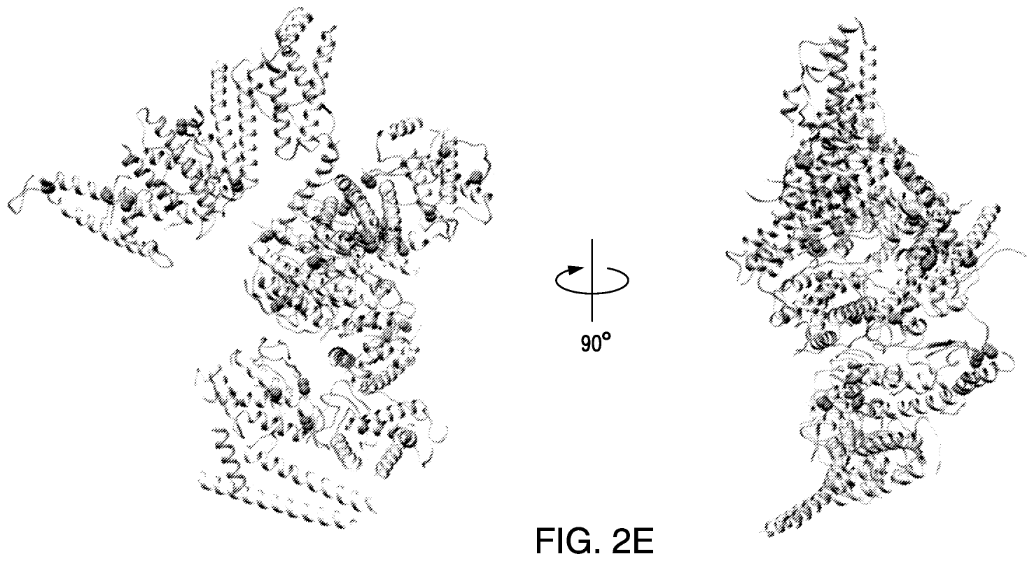

The inventors have discovered that VAR2CSA is primarily composed of α-helices and extensive loops that adopt an overall shape resembling the number 7 (FIG. 1c). The region composed of DBL2X to ID3 forms a relatively stable core, while DBL5ε-DBL6ε forms a flexible arm and DBL1X exhibits some structural flexibility. The 6 individual DBL domains of VAR2CSA adopt the classical DBL domain fold, consisting of an α-helical core decorated by extensive loops (FIG. 2a). The individual domains interact in an interwoven manner to stabilize the compact tertiary structure (FIG. 1c, d). DBL4ε, is located at the center of VAR2CSA and unites the whole structure by directly interacting with all the other domains except DBL1X and DBL5ε (FIG. 1c, d). DBL1X and DBL5ε are connected to DBL4ε via the NTS and ID3, respectively (FIG. 1c, d). The NTS (residues 32-49) is a twisted loop surrounding DBL1X and serves as the mortar holding together DBL1X and DBL4ε, with high conservation among diverse VAR2CSA strains (FIG. 2a-c). ID3 is a long helix that closely interacts with ID2 and connects DBL5ε with the core (FIG. 2a, d). A total of 31 pairs of disulfide bridges were identified in the final model (FIG. 2e and Table 3, below).

When bound by VAR2CSA, the inventors have further discovered that CSA traverses the core domain by binding within two channels, which are referred to herein as the “major CSA binding channels” and the “minor CSA binding channels”.

Aspects of the disclosure comprises, consists essentially of, or consists of an immunogen polypeptide, wherein the immunogen polypeptide comprises, consists essentially of, or consists of portions of a wildtype VAR2CSA protein, for example a VAR2CSA protein obtained from P. falciparum strain N5F4 or FCR3. The disclosed immunogen polypeptides may encompass important regions of VAR2CSA for binding, conservation and immunogenicity as informed by the structure of VAR2CSA as elucidated by the inventors. For example, the immunogen polypeptide may include regions of wild-type VAR2CSA associated with CSA binding.

The sequence of the disclosed immunogen polypeptides may be based on the sequence of any suitable VAR2CSA variant. For example, the sequence of the immunogen polypeptide may be derived from the sequences of the VAR2CSA protein from P. falciparum strain 7G8 (SEQ ID NO: 55), the VAR2SCA protein from P. falciparum strain HB3 (SEQ ID NO:56), the VAR2SCA protein from P. falciparum strain CD01 (SEQ ID NO: 57), the VAR2SCA protein from P. falciparum strain Dd2 (SEQ ID NO: 58), the VAR2SCA protein from P. falciparum strain GA01 (SEQ ID NO: 59), the VAR2SCA protein from P. falciparum strain GB4 (SEQ ID NO:60), the VAR2SCA protein from P. falciparum strain KE01 (SEQ ID NO: 61), the VAR2SCA protein from P. falciparum strain KHO1 (SEQ ID NO: 62), the VAR2SCA protein from P. falciparum strain ML01 (SEQ ID NO: 63), the VAR2SCA protein from P. falciparum strain SNO1 (SEQ ID NO: 64), the VAR2SCA protein from P. falciparum strain TGO1 (SEQ ID NO: 65), and/or the VAR2SCA protein from P. falciparum strain GN01 (SEQ ID NO: 66).

Included in the disclosure are immunogen polypeptides that are functional variants of portions of a wildtype VAR2CSA protein. The term “functional variant,” as used herein, refers to a polypeptide, or protein having substantial or significant sequence identity or similarity to a parent polypeptide, which functional variant retains the biological activity of the immunogen polypeptide of which it is a variant. Functional variants encompass, for example, those variants of the immunogen polypeptides described herein that retain the ability to bind CSA to a similar extent, the same extent, or to a higher extent, as the parent immunogen polypeptide. In reference to the parent polypeptide, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent polypeptide.

In general, a functional variant according to the disclosure can, for example, comprise the amino acid sequence of the parent polypeptide with at least one conservative amino acid substitution. Alternatively or additionally, the functional variants can comprise the amino acid sequence of the parent polypeptide with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent polypeptide.

The immunogen polypeptide may also comprise functional portions of a wildtype VAR2CSA protein. In general, a “functional portion” according to the disclosure is part or fragment of a parent polypeptide that retains the biological activity of the parent polypeptide. Functional portions encompass, for example, those parts of a wildtype VAR2CSA protein that bind CSA. In reference to the parent polypeptide, the functional portion can comprise, for instance, about 10%, about 25%, about 30%, about 50%, about 70%, about 80%, about 90%, about 91%, about 92%, about 93%, about 94%, about 95%, about 96%, about 97%, about 98%, about 99% or more of the parent polypeptide.

The functional portion can comprise additional amino acids at the amino or carboxy terminus of the portion, or at both termini, which additional amino acids are not found in the amino acid sequence of the parent polypeptide. Desirably, the additional amino acids do not interfere with the biological function of the functional portion, e.g., binding to CSA. More desirably, the additional amino acids enhance the biological activity, as compared to the biological activity of the parent polypeptide.

In aspects of the disclosure, the immunogen polypeptide comprises, consists essentially of, or consists of a) all or portions of the major CSA binding channel of VAR2CSA; b) all or portions of the minor CSA binding channel of VAR2CSA; and/or all or portions of the arm segment of VAR2CSA, or functional portions or variants thereof.

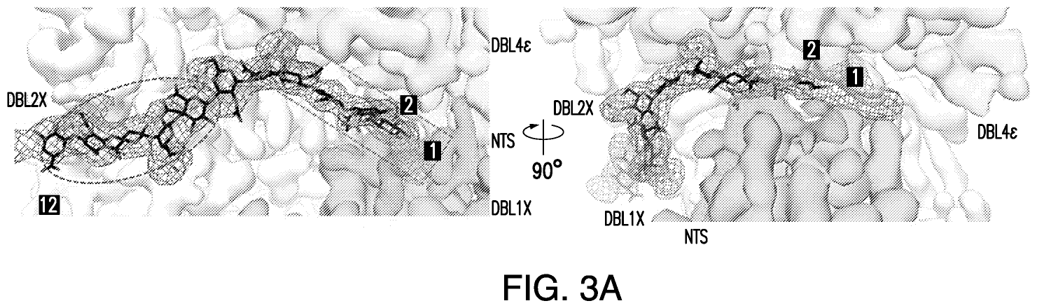

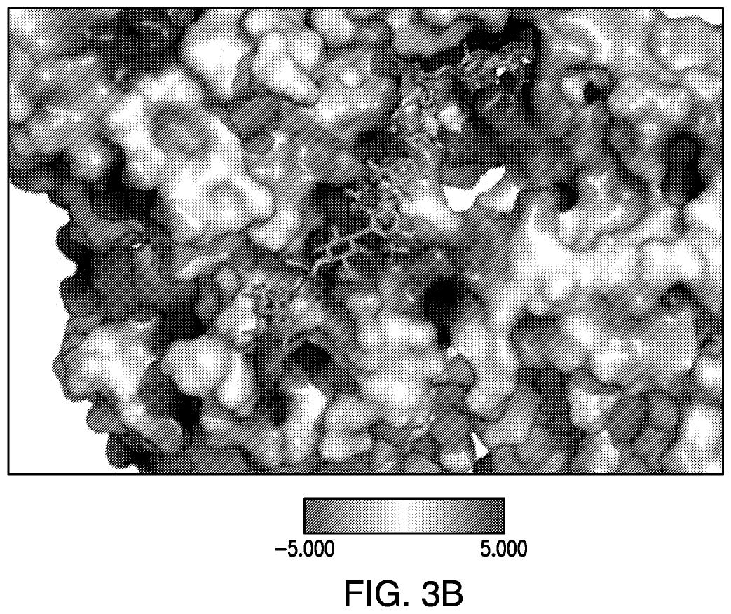

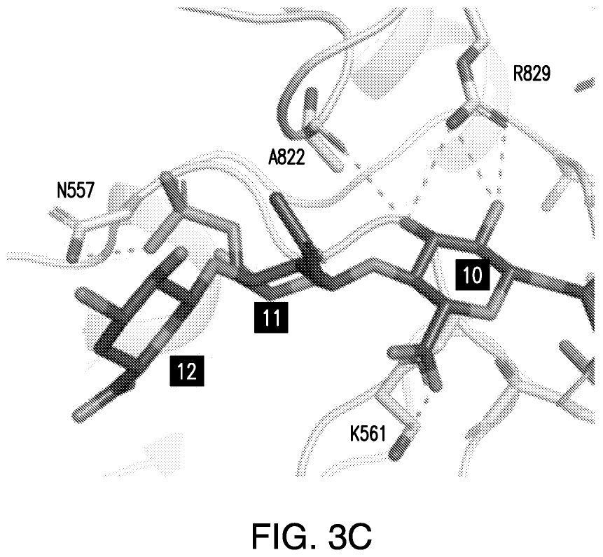

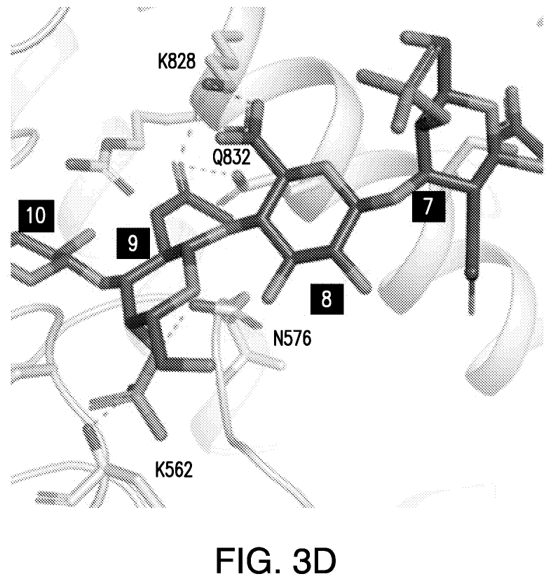

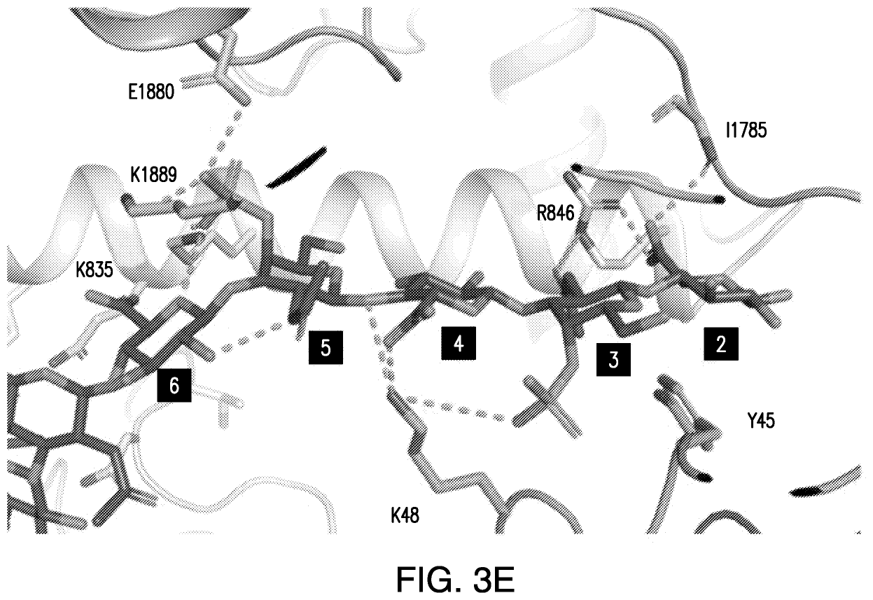

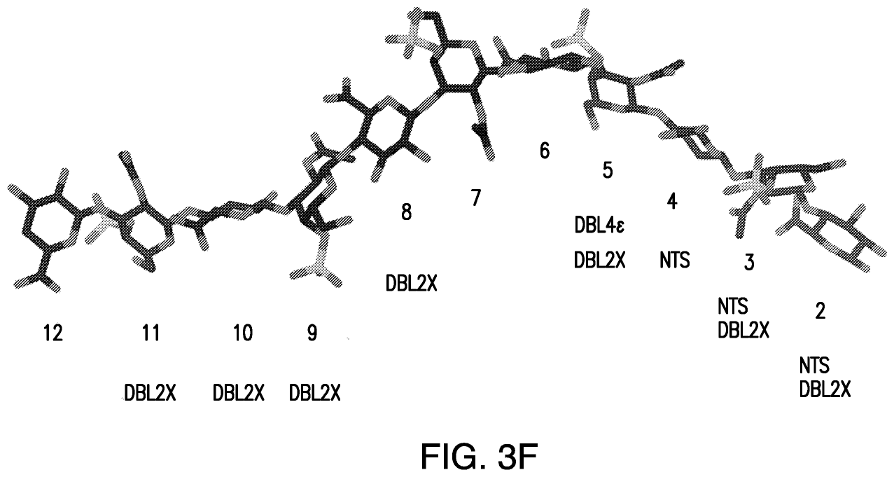

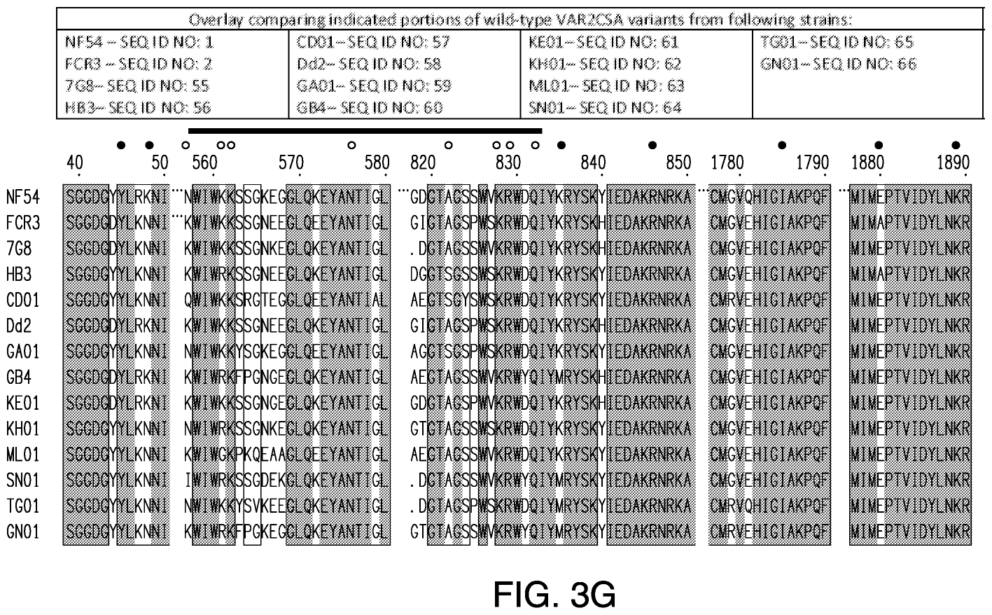

The “major CSA binding channel” refers to a charged channel that is formed by NTS, DBL1X, DBL2X and DBL4ε. (FIG. 3a, b). The major binding channel can be separated into two non-continuous CSA-binding sites (FIG. 3a). The first binding site (“major binding site 1”) is located on the surface of DBL2X and binds CSA residues BDP-8 to ASG-11 (FIG. 3a, c, d). The sulfate group of ASG-11 forms hydrogen bonds with N557 while BDP-10 has interactions with R829, K561 and the main chain of A822 (FIG. 3c). ASG-9 forms multiple hydrogen bonds with K562, N576, K828 and Q832 (FIG. 3d). The interaction of CSA with major binding site 1 is further strengthened by the hydrogen bonds between BDP-8 and K828 (FIG. 3d).

The second binding site within the major CSA binding channel (“major binding site 2”) lies deep in the hole of the funnel-shaped channel and is surrounded by NTS, DBL1X, DBL2X and DBL4ε (FIG. 3a,b). Multiple hydrogen bonds are also formed in this region: ASG-5 with K835, E1880 and K1889; BDP-4 with K48; ASG-3 with K48 and R846; BDP-2 with R846 and the main chain of 11785 (FIG. 3e). Y45 further stabilizes the interaction by packing tightly with BDP-2 (FIG. 3e). BDP-6 and ASG-7 do not exhibit direct interactions with CSA and without being bound by a particular theory, may serve to link the two major binding sites together (FIG. 3f).

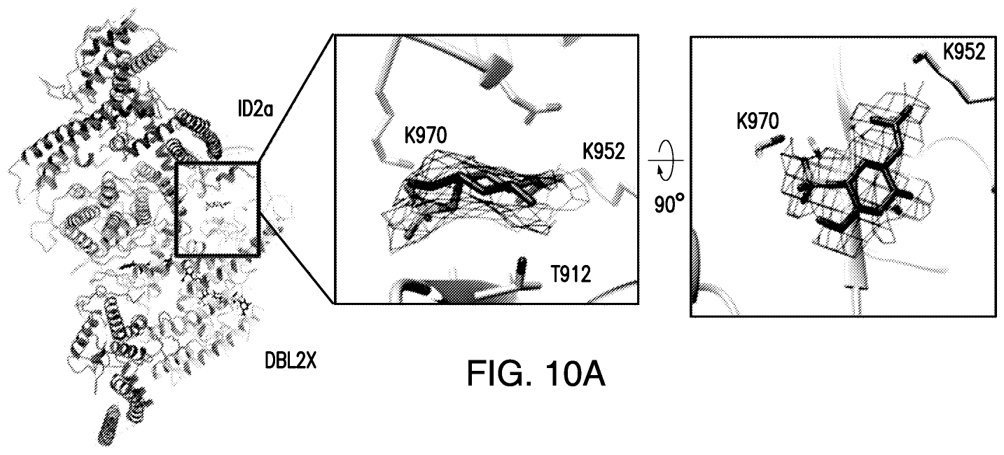

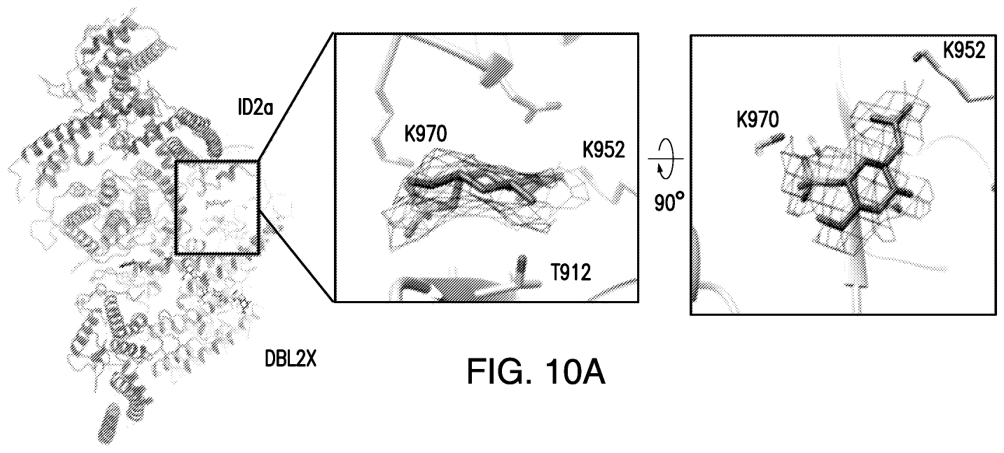

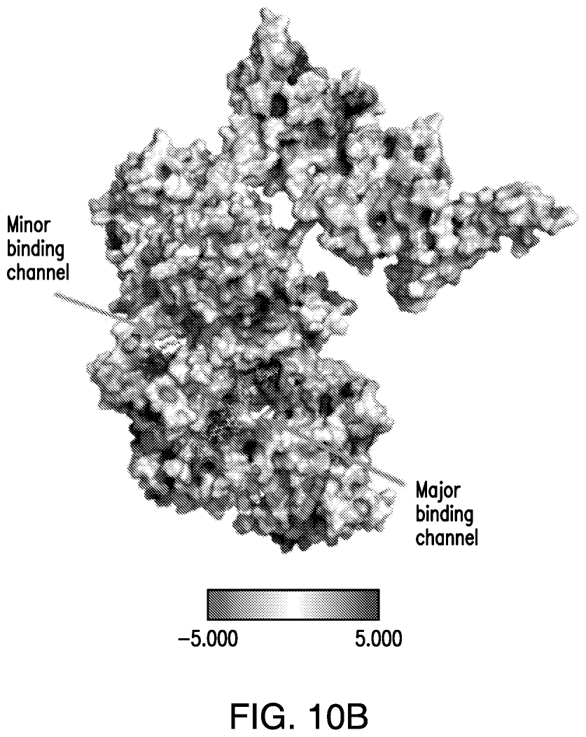

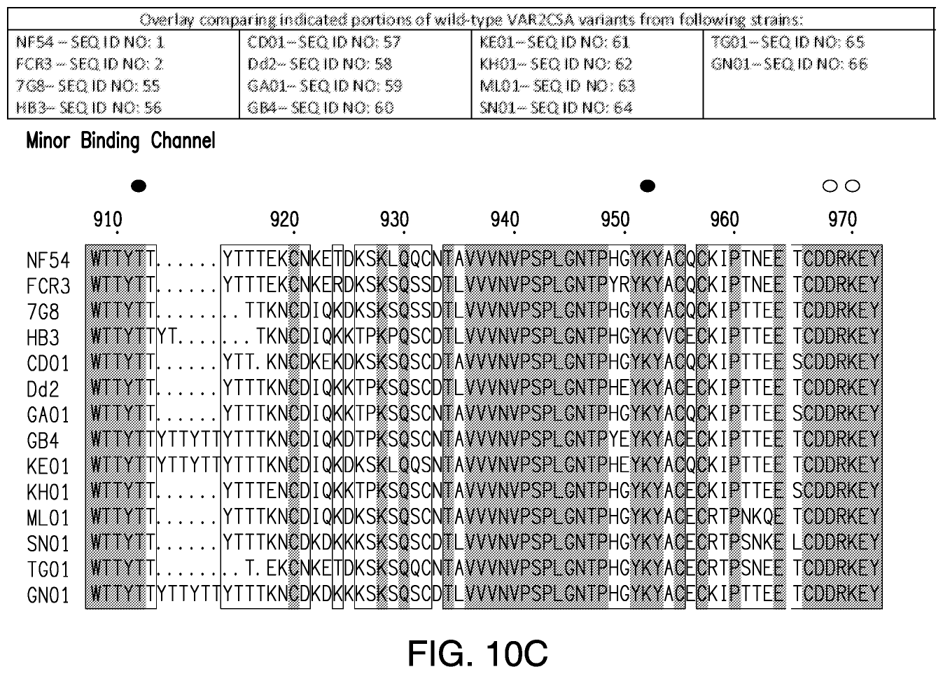

The “minor CSA binding channel” refers to an area of weak density that the inventors have identified in a separate region of VAR2CSA. The minor CSA binding channel forms a potential second binding site (FIG. 10a). The minor binding channel is made up of the residues from the C-terminus of DBL2X and N-terminus of ID2a, two regions previously implicated in CSA binding. Similar to the major binding channel, the minor channel is rich in positively charged residues (FIG. 10b).

The “arm segment VAR2CSA” refers to the flexible arm region of VAR2CSA, distinct from the core region, which is formed by DBL5ε-DBL6ε (see, e.g., FIG. 1C, 8F, 8I.)

In aspects, the immunogen polypeptide comprises NTS, DBL1X, ID1, DBL2X, ID2a, ID2b, DBL3X, DBL4ε, ID3, DBL5ε and DBL6ε. In aspects if the disclosure, this configuration may be referred to as “full length”. In aspects, the immunogen polypeptide comprises NTS, DBL1X, ID1, DBL2X, ID2a, ID2b, DBL3X, DBL4ε, and ID3. In aspects of the disclosure, this configuration may be referred to as the “core”. In aspects, the immunogen polypeptide comprises NTS, DBL1X, DBL2X, ID2a, ID2b, DBL3X, DBL4ε, and ID3. In aspects of the disclosure, this configuration may be referred to as the “core min”. In aspects, the immunogen polypeptide comprises NTS, DBL1X, ID1, DBL2X, ID2a, ID2b, DBL4ε, and ID3. In aspects of the disclosure, this configuration may be referred to as the “major and minor channels”. In aspects, the immunogen polypeptide comprises NTS, DBL1X, DBL2X, ID2a, ID2b DBL4ε, and ID3. In aspects of the disclosure, this configuration may be referred to as the “major and minor channels min”. In aspects, the immunogen polypeptide comprises NTS, DBL1X, ID1, DBL2X, DBL4ε, and ID3. In aspects of the disclosure, this configuration may be referred to as the “major channel”. In aspects, the immunogen polypeptide comprises NTS, DBL1X, DBL2X, DBL4ε, and ID3. In aspects of the disclosure, this configuration may be referred to as the “Major channel min”. In aspects, the immunogen polypeptide comprises DBL5ε and DBL6ε. In aspects of the disclosure this configuration may be referred to as the “Arm”.

In aspects, the immunogen polypeptide comprises, consists essentially of, or consists of any one of SEQ ID NOS: 3-54. SEQ ID NOS: 3-54 are described in Table 1.

TABLE 1

| SEQ ID NO: | P. falciparum strain | Configuration Description |
|---|---|---|
| 3 | NF54 | Major and minor channels |
| 4 | FCR3 | Major and minor channels |
| 5 | NF54 | Core |
| 6 | FCR3 | Core |
| 7 | NF54 | Arm |
| 8 | FCR3 | Arm |
| 9 | NF54 | Major channel |
| 10 | FCR3 | Major Channel |
| 11 | NF54 | Major channel min |
| 12 | FCR3 | Major channel min |
| 13 | NF54 | DBL2 |
| 14 | FCR3 | DBL2 |
| 15 | NF54 | Core min |
| 16 | FCR3 | Core min |
| 17 | NF54 | Major and minor channels min |
| 18 | FCR3 | Major and minor channels min |
| 19 | 7G8 | Core |
| 20 | HB3 | Core |
| 21 | CD01 | Core |
| 22 | DD2 | Core |
| 23 | GA01 | Core |
| 24 | GB4 | Core |
| 25 | KE01 | Core |
| 26 | KH01 | Core |
| 27 | ML01 | Core |
| 28 | SN01 | Core |
| 29 | TG01 | Core |
| 30 | GN01 | Core |
| 31 | 7G8 | Major and minor channels |
| 32 | HB3 | Major and minor channels |
| 33 | CD01 | Major and minor channels |
| 34 | DD2 | Major and minor channels |
| 35 | GA01 | Major and minor channels |
| 36 | GB4 | Major and minor channels |
| 37 | KE01 | Major and minor channels |
| 38 | KH01 | Major and minor channels |
| 39 | ML01 | Major and minor channels |
| 40 | SN01 | Major and minor channels |
| 41 | TG01 | Major and minor channels |
| 42 | GN01 | Major and minor channels |
| 43 | 7G8 | Major channel |
| 44 | HB3 | Major channel |
| 45 | CD01 | Major channel |
| 46 | DD2 | Major channel |
| 47 | GA01 | Major channel |
| 48 | GB4 | Major channel |
| 49 | KE01 | Major channel |
| 50 | KH01 | Major channel |
| 51 | ML01 | Major channel |
| 52 | SN01 | Major channel |
| 53 | TG01 | Major channel |
| 54 | GN01 | Major channel |

In aspects, the immunogen polypeptide comprises, consists essentially of, or consists of a polypeptide having at least about 80% identity with any one of SEQ ID NOs: 3-54. In aspects, the immunogen polypeptide comprises, consists essentially of, or consists of a polypeptide having at least about 85% identity with any one of SEQ ID NOs: 3-54. In aspects, the immunogen polypeptide comprises, consists essentially of, or consists of a polypeptide having at least about 90% identity with any one of SEQ ID NOs: 3-54. In aspects, the immunogen polypeptide comprises, consists essentially of, or consists of a polypeptide having at least about 90%, about 91%, about 92%, about 93%, about 94%, about 95%, about 96%, about 97%, about 98%, about 99%, or more identity with any one of SEQ ID NOs: 3-54.

“Conserved CSA binding channel residues” refers to residues within the major and minor CSA binding channels that are highly conserved among different VAR2CSA alleles and among different strains of P. falciparum. Conserved CSA binding channel residues, discovered by the inventors, are presented in FIG. 3g and FIG. 10c. Without being bound by a particular theory, the high conservation of the residues within both channels that directly bind CSA indicate these residues are under selective pressure to be maintained across strains.

In aspects, “conserved CSA binding channel residues” refers to one or more amino acids corresponding to the following amino acids in the major CSA binding channel of full length VAR2CSA from P. falciparum strain NF54 (SEQ ID NO:1): 44-46, 47-49, 556-558, 560-563, 575-577, 821-823, 827-836, 1784-1786, 1879-1881, and 1888-1890. In aspects, the “conserved CSA binding channel residues” refers to one or more amino acids corresponding to the following amino acids in the major CSA binding channel of full length VAR2CSA from P. falciparum strain NF54 (SEQ ID NO:1): 45, 48, 557, 561, 562, 576, 822, 828, 829, 832, 835, 1785, 1880, and 1889. In aspects, “conserved CSA binding channel residues” refers to one or more amino acids corresponding to the following amino acids in the minor CSA binding channel of full length VAR2CSA from P. falciparum strain NF54 (SEQ ID NO:1): 911-913, 951-953, and 967-971. In aspects, the “conserved CSA binding channel residues” refers to one or more amino acids corresponding to the following amino acids in the minor CSA binding channel of full length VAR2CSA from P. falciparum strain NF54 (SEQ ID NO:1): 912, 952, 968, and 970.

One skilled in the art will recognize that the above listed amino acid numbers refer to the VAR2CSA protein expressed by the NF54 strain of P. falciparum. One skilled in the art will further understand that different strains of P. falciparum may express different VAR2CSA alleles or variants. One skilled in the art will be able to identify the amino acids in such different VAR2CSA alleles or variants that correspond with the above listed amino acids in the NF54 VAR2CSA protein by, for example, analyzing sequence and crystallographic data. For example, corresponding amino acids may be identified by performing sequence alignments comparing the sequence of the NF54 VAR2CSA to other VAR2CSA alleles or variants. Examples of such alignments are depicted in FIGS. 3G and 10C.

Amino acid substitutions of the disclosed immunogen polypeptides are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The immunogen polypeptide can consist essentially of the specified amino acid sequence or sequences described herein, such that other components, e.g., other amino acids, do not materially change the biological activity of the functional variant.

In aspects, the immunogen polypeptide of the disclosure (including functional portions and functional variants) can be of any length, i.e., can comprise any number of amino acids, provided that the immunogen polypeptides (or functional portions or functional variants thereof) retain their biological activity, e.g., the ability to specifically bind to antigen, detect diseased cells in a mammal, or treat or prevent disease in a mammal, etc. For example, the immunogen polypeptide can be about 50 to about 5000 amino acids long, such as 50, 70, 75, 100, 125, 150, 175, 200, 300, 400, 500, 600, 700, 800, 900, 1000 or more amino acids in length.

The immunogen polypeptide of aspects of the disclosure (including functional portions and functional variants of the disclosure) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

In aspects, the immunogen polypeptides of the disclosure can be glycosylated, amidated, carboxylated, phosphorylated, esterified, N-acylated, cyclized via, e.g., a disulfide bridge, or converted into an acid addition salt and/or optionally dimerized or polymerized, or conjugated.

The immunogen polypeptides of the disclosure may be conformationally stabilized in any of a number of ways, including for, example, the use of disulfide bonds or lactam bridges.

The immunogen polypeptides of the present disclosure can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques. Both post-translational modifications and chemical modification techniques are well described in the art. Modifications can occur anywhere in a polypeptide, including the peptide backbone, the amino acid side-chains and the amino or carboxyl termini. The same type of modification may be present in the same or varying degrees at several sites in a given polypeptide, and a given polypeptide may contain many types of modifications. Polypeptides may be branched, for example, as a result of ubiquitination, and they may be cyclic, with or without branching. Cyclic, branched, and branched cyclic polypeptides may result from posttranslational natural processes or may be made by synthetic methods.

In aspects of the disclosure, the immunogen polypeptides may be modified. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins-Structure and Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., 1990, Meth. Enzymol. 182:626-646; Rattan et al., 1992, Ann. 1VY Acad. Sci. 663:48-62).

In aspects, the immunogen polypeptides of the disclosure (including functional portions and functional variants thereof) can be obtained by methods known in the art. The immunogen polypeptides may be made by any suitable method of making polypeptides or proteins, including de novo synthesis. Also, the immunogen polypeptides can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, NY 2012. Further, portions of some of the immunogen polypeptides of the disclosure (including functional portions and functional variants thereof) can be isolated and/or purified from a source, such as a plant, a bacterium, an insect, a mammal, e.g., a rat, a human, etc. Methods of isolation and purification are well-known in the art. Alternatively, the immunogen polypeptides described herein (including functional portions and functional variants thereof) can be commercially synthesized by companies, such as Synpep (Dublin, CA), Peptide Technologies Corp. (Gaithersburg, MD), and Multiple Peptide Systems (San Diego, CA). In this respect, the disclosed immunogen polypeptides can be synthetic, recombinant, isolated, and/or purified. In aspects, the immunogen polypeptides are prepared by recombinant production using overexpression of desired nucleic acid sequences (i.e., sequences encoding the immunogen polypeptides) from plasmids in mammalian cell culture expression systems such as, e.g., Expi293.

In aspects of the disclosure, the immunogen polypeptide is isolated or purified. “Isolated” refers to the removal of a substance (e.g., a protein or nucleic acid) from its natural environment. “Purified” means that a given substance (e.g., a protein or nucleic acid), whether one that has been removed from nature (e.g., genomic DNA and mRNA) or synthesized (e.g., cDNA) and/or amplified under laboratory conditions, has been increased in purity, wherein “purity” is a relative term, not “absolute purity.” It is to be understood, however, that nucleic acids and proteins may be formulated with diluents or adjuvants and still for practical purposes be isolated. For example, proteins typically are mixed with an acceptable carrier or diluent when used for introduction into cells.

In other aspects of the disclosure, the immunogen polypeptides may not be isolated. In aspects of the disclosure, the immunogen polypeptides may not be purified.

When the immunogen polypeptides, are in the form of a salt, preferably, the polypeptides are in the form of a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

In aspects of the disclosure, the immunogen polypeptide can consist essentially of the immunogen polypeptides as described herein, such that other components, e.g., other amino acids, do not materially change the biological activity of the immunogen polypeptide. In other aspects of the disclosure, the immunogen polypeptide can consist of the immunogen polypeptides as described herein. Pharmaceutical Compositions

The disclosed immunogen polypeptides can be formulated into a composition, such as a pharmaceutical composition. In this regard, aspects of the disclosure provide a pharmaceutical composition comprising any of the disclosed immunogen polypeptides described herein and a pharmaceutically acceptable carrier. The pharmaceutical composition can comprise an disclosed immunogen polypeptide in combination with other pharmaceutically active agents or drugs, such as chemotherapeutic agents. A pharmaceutical composition according to the present disclosure may comprise immunogen polypeptides comprising immunogen polypeptides based on one or more wild type VAR2CSA variants.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which has no detrimental side effects or toxicity under the conditions of use. The pharmaceutical carrier can be water.

The choice of carrier will be determined in part by the particular disclosed immunogen polypeptides, as well as by the particular method used to administer the disclosed immunogen polypeptides. Accordingly, there are a variety of suitable formulations of the pharmaceutical composition of the disclosure. Methods for preparing administrable (e.g., parenterally administrable) compositions are known or apparent to those skilled in the art and are described in more detail in, for example, Remington: The Science and Practice of Pharmacy, Pharmaceutical Press; 22nd ed. (2012).

The disclosed immunogen polypeptides may be administered in any suitable manner. Preferably, the immunogen polypeptides are administered by injection, (e.g., subcutaneously, intravenously, intratumorally, intraarterially, intramuscularly, intradermally, interperitoneally, or intrathecally). In aspects, the disclosed immunogen polypeptides are administered intravenously. In other aspects, the disclosed immunogen polypeptides are administered subcutaneously. A suitable pharmaceutically acceptable carrier for the disclosed immunogen polypeptides for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In aspects, the pharmaceutically acceptable carrier is supplemented with human serum albumen.

The compositions may contain any suitable amount of the disclosed immunogen polypeptides. For example, the compositions may contain from about 0.5% to about 25% by weight of the immunogen polypeptide in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Alternatively, the composition can be modified into a depot form, such that the manner in which the vaccine protein is released into the body to which it is administered is controlled with respect to time and location within the body (see, for example, U.S. Pat. No. 4,450,150). Depot forms of immunogen polypeptides can be, for example, an implantable composition comprising the vaccine proteins and a porous or non-porous material, such as a polymer, wherein the immunogen polypeptide is encapsulated by or diffused throughout the material and/or degradation of the non-porous material. The depot is then implanted into the desired location within the body and the immunogen polypeptides are released from the implant at a predetermined rate.

Aspects of the disclosure include compositions comprising multimers of the disclosed immunogen polypeptides. As used herein, “multimer” refers to polymer comprising 2 or more immunogen polypeptides. The term multimer includes immunogen peptide dimers, immunogen peptide trimers, immunogen peptide tetramers and higher immunogen peptide multimers. Multimers of the immunogen polypeptides may have improved binding characteristics, e.g. to CSA, and may have improved immunogenicity relative to immunogen polypeptide monomers.

Aspects of the disclosure include compositions comprising the disclosed immunogen polypeptides in nanoparticle form. As used herein, “nanoparticle” refers to a particle having a nanoscale size in at least one dimension (e.g., one, two, or three dimensions), e.g., about 1 nm, about 10 nm, or about 100 nm. Immunogen polypeptides formulated as nanoparticles may have improved binding characteristics, e.g. to CSA, and may have improved immunogenicity relative to immunogen polypeptides not formulated as nano particles.

In aspects, the composition may further comprise an additional therapeutic agent. The additional therapeutic agents can be, for example, plant, fungal, or bacterial molecules (e.g., a protein toxin), small molecule chemotherapeutics, or biological therapeutics. In aspects, the additional therapeutic agents comprise chemotherapeutic agents or anticancer agents. Suitable chemotherapeutic agents or other anticancer agents for use in accordance with the disclosure include but are not limited to tyrosine kinase inhibitors (genistein), biologically active agents (TNF, of tTF), radionuclides (1311, 90Y, 111In, 211At, 32P and other known therapeutic radionuclides), adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus) and derivatives, mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, vinblastine, vincristine, vinorelbine, taxol, combretastatins, discodermolides, and transplatinum. Accordingly, suitable chemotherapeutic agents for use in accordance with disclosure include, without limitation, antimetabolites (e.g., asparaginase), antimitotics (e.g., vinca alkaloids), DNA damaging agents (e.g., cisplatin), proapoptotics (agents which induce programmed-cell-death or apoptosis) (e.g, epipodophyllotoxins), differentiation inducing agents (e.g., retinoids), antibiotics (e.g., bleomycin), and hormones (e.g., tamoxifen, diethylstilbestrol).

Further, suitable chemotherapeutic agents for use in accordance with the disclosure include antiangiogenesis agents (angiogenesis inhibitors) such as, e.g., INF-alpha, fumagillin, angiostatin, endostatin, thalidomide, and the like. “Other anticancer agents” also include, without limitation, biologically active polypeptides, antibodies, lectins, and toxins. Suitable antibodies for use in accordance with the disclosure include, without limitation, conjugated (coupled) or unconjugated (uncoupled) antibodies, monoclonal or polyclonal antibodies, humanized or unhumanized antibodies, as well as Fab′, Fab, or Fab2 fragments, single chain antibodies and the like.

In aspects, the disclosure comprises, consists essentially of, or consists of a pharmaceutical composition comprising an additional therapeutic agent coupled, conjugated or fused to an immunogen polypeptide. The chemotherapeutic agent can be coupled to the immunogen polypeptide using any suitable method. Preferably, the additional therapeutic agent is chemically coupled to the compound via covalent bonds including, for example, disulfide bonds. Other methods for coupling or conjugation of suitable therapeutics, chemotherapeutics, ra.dionuclides, polypeptides, and the disclosed immunogen polypeptides are well described in the art. Any suitable method can he used in accordance with the disclosure to form the immunogen polypeptide conjugates. For example, without limitation, free amino groups in the immunogen polypeptide, such the epsilon-amino group of lysine, can be conjugated with reagents such as carbodiimides or heterobifunctional agents. In aspects, e.g., sulfhydryl groups can be used for conjugation. In addition, sugar moieties bound to immunogen polypeptides, can be oxidized to foam aldehydes groups useful in a number of coupling procedures known in the art. The conjugates formed in accordance with the disclosure can be stable in vivo or labile, such as enzymatically degradable tetrapeptide linkages or acid-labile cis-aconityl or hydrazone linkages.

In aspects of the disclosure, the pharmaceutical composition can consist essentially of the pharmaceutical compositions as described herein, such that other components, e.g., other excipients, do not materially change the characteristics of the pharmaceutical composition. In other aspects of the disclosure, the pharmaceutical composition can consist of the pharmaceutical compositions as described herein.

### Methods of Vaccination

In aspects, the disclosure comprises, consists essentially of, or consists of a method of vaccinating a subject against malaria. The method comprises, consists essentially of, or consists of obtaining an isolated or purified immunogen polypeptide.

Immunogen polypeptides suitable for the methods of vaccinating are described above. Such immunogen polypeptides comprise, consist essentially of, or consist of a) all or portions of the major CSA binding channel of VAR2CSA; and/or b) all or portions of the minor CSA binding channel of VAR2CSA. In aspects the immunogen polypeptide comprises, consists essentially of, or consists of all or portions of the major CSA binding channel. In aspects the immunogen polypeptide comprises, consists essentially of, or consists of all or portions of the minor CSA binding channel. In aspects the immunogen polypeptide comprises, consists essentially of, or consists of all or portions of the arm segment. The immunogen polypeptide may comprise one or more conserved VAR2CSA binding channel residues as described above. In certain aspects, the conserved VAR2CSA binding residues are located in the major CSA binding channel of VAR2CSA.

In aspects, the method of vaccinating a subject against malaria comprises, consists essentially of, or consists of obtaining an immunogen polypeptide having at least 80% identity with any one of SEQ ID NOs: 3-54. In aspects, the immunogen polypeptide comprises, consists essentially of, or consists of a polypeptide having at least 85% identity with any one of SEQ ID NOs: 3-54. In aspects, the immunogen polypeptide comprises, consists essentially of, or consists of a polypeptide having at least 90% identity with any one of SEQ ID NOs: 3-54. In aspects, the immunogen polypeptide comprises, consists essentially of, or consists of a polypeptide having at least 95% identity with any one of SEQ ID NOs: 3-54. In some aspects the immunogen polypeptide comprises, consists essentially of, or consists of any one of SEQ ID Nos: 3-54.

In aspects, the method of vaccinating a subject against malaria comprises, consists essentially of, or consists of administering an effective amount of a pharmaceutical composition. Examples of suitable pharmaceutical formulations are provided above. For the purposes of vaccination, compositions can be administered in dosages and by techniques well known to those skilled in the medical or veterinary arts, taking into consideration such factors as the age, sex, weight, species and condition of the recipient subject, and the route of administration.

In aspects, the method of vaccinating a subject against malaria comprises, consists essentially of, or consists of administering an effective amount of the composition to the subject. In the context of methods of vaccinating a subject against malaria, “effective amount” refers to the amount of a composition necessary to provide at least a partial protective effect against natural malaria infection, as evidenced by a reduction in the mortality and morbidity associated with natural malaria infection.

The effective dosage of the composition made according to the present disclosure will depend on the species, age, size, vaccination history, and health status of the subject to be vaccinated. Other factors like immunogen polypeptide concentration, and route of administration (i.e., subcutaneous, intradermal, oral, intramuscular or intravenous administration) will also impact the effective dosage. One skilled in the art will be able to readily determine the effective dosage based on the antigen concentration of the vaccine, the route of administration, and the age and condition of the subject. In aspects, each batch of antigen may be individually calibrated. Alternatively, methodical immunogenicity trials of different dosages, as well as LD5o studies and other screening procedures can be used to determine the effective dosage. Such methods are well known to those skilled in the art and require no undue experimentation. The effective dose of the immunogen polypeptide also will be determined by the existence, nature and extent of any adverse side effects that might accompany the administration of a particular immunogen polypeptide. By way of example, and without limitation, the dose of the immunogen polypeptide can be about 0.001 to about 1000 mg/kg body weight of the subject being treated/day, from about 0.01 to about 10 mg/kg body weight/day, about 0.01 mg to about 1 mg/kg body weight/day.

The route of administration of the pharmaceutical composition in accordance with the methods of vaccination may be percutaneous, via mucosal administration (e.g., oral, nasal, anal, vaginal) or via a parenteral route (intradermal, intramuscular, subcutaneous, intravenous, or intraperitoneal). Vaccine compositions can be administered alone, or can be co-administered or sequentially administered with other treatments or therapies.

In aspects, forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray or mixed in food and/or water or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired.

In aspects, the administration of the composition elicits an immune response in the subject. As used herein, the term “immune response” refers to a response elicited in a subject. An immune response may refer to cellular immunity; humoral immunity or both. The present disclosure also contemplates a response limited to a part of the immune system. In aspects, the immune response elicited by the administration of the disclosed pharmaceutical compositions is sufficient to provide at least a partial protective effect against natural malaria infection, as evidenced by a reduction in the mortality and morbidity associated with natural malaria infection. In aspects, the immune response is sufficient to provide at least a partial protective effect against natural placental malaria infection, as evidenced by a reduction in the mortality and/or morbidity associated with natural placental malaria infection. In aspects, the reduction in mortality and morbidity is strain transcending. In other words, aspects of the disclosed methods of vaccination provide a protective effect against natural infection by more than one strain of P. falciparum.

In aspects of the disclosure, the methods of vaccination can consist essentially of the methods of vaccination as described herein, such that any variation does not materially change the characteristics of the method. In other aspects of the disclosure, the methods of vaccination consist of the methods of vaccination as described herein.

### Methods of Treatment

In aspects, the disclosure provides a method for treating a disease in a subject. The subject may be any organism. In certain aspects the subject is a mammal. In preferred aspects the subject is human.

In aspects, the disease may include abnormal conditions of proliferation, tissue remodeling, hyperplasia, and exaggerated wound healing in bodily tissue (e.g., soft tissue, connective tissue, bone, solid organs, blood vessel and the like). In aspects, the disclosure comprises, consists essentially of, or consists of a method of treating or preventing cancer.

Non-limiting examples of specific types of cancers include cancer of the head and neck, eye, skin, mouth, throat, esophagus, chest, bone, lung, colon, sigmoid, rectum, stomach, prostate, breast, ovaries, uterus (e.g., endometrium), kidney, liver, pancreas, brain, intestine, heart or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, meningioma, melanoma, neuroblastoma, retinoblastoma, a blood-born tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia, chronic lymphocytic leukemia, hairy cell leukemia, or multiple myeloma. See, e.g., Harrison's Principles of Internal Medicine, Eugene Braunwald et al., eds., pp.491 762 (15th ed. 2001).

In aspects the cancer is a CSA expressing cancer. A non-limiting list of cancers known to express CSA is provided as Table 2.

TABLE 2

| Disease/cancer line studied | Potential usage | Reference |
|---|---|---|
| Colorectal cancer | Treatment, Diagnosis | Mette Ø. Agerbaek, et al., Nature Communications, 2018; Nicolai T. |
| (CDLO205, HT-29, SW480) | | Sand, et al., Int. J. Mol. Sci. 2020. |
| Lung cancer | Treatment, Diagnosis | Nicolai T. Sand, et al., Int. J. Mol. Sci. 2020. |
| (A549) | | |
| Breast cancer | Treatment, Diagnosis | Mette Ø. Agerbaek, et al., Nature Communications, 2018; Nicolai T. |
| (MDA-MB-231, MCF7, Hs578T, SK-BR-3) | | Sand, et al., Int. J. Mol. Sci. 2020. |
| Prostate cancer | Treatment, Diagnosis | Mette Ø. Agerbaek, et al., Nature Communications, 2018; Nicolai T. |
| (LNCaP, PC3, DU145) | | Sand, et al., Int. J. Mol. Sci. 2020. |
| Osteosarcoma | Treatment, Diagnosis | Mette Ø. Agerbaek, et al., Nature Communications, 2018 |
| (U2OS) | | |
| Melanoma | Treatment, Diagnosis | Mette Ø. Agerbaek, et al., Nature Communications, 2018 |
| (C32) | | |
| Diffuse gliomas/brain cancer | Diagnosis | Sara R. Beng-Christensen, et al., Cells, 2019 |
| (KNS-42, Res259, U87 mg, and U118 mg) | | |

The terms “treating,” and “preventing,” as used herein, refer to curative therapy, prophylactic therapy, or preventative therapy. An example of “preventing” or “preventative therapy” is the prevention or lessening of the chance of a targeted disease (e. g., cancer or other proliferative disease), or related condition thereto, occurring in a subject. Those in need of such preventative therapy may include those already with the disease or condition as well as those prone to have the disease or condition to be prevented. The terms “treating,” “treatment,” “therapy,” and “therapeutic treatment” as used herein also describe the management and care of a subject (e.g. a human) for the purpose of combating a disease, or related condition, and includes the administration of a composition to alleviate the symptoms, side effects, or other complications of the disease, condition. Therapeutic treatment for cancer includes, but is not limited to, surgery, chemotherapy, radiation therapy, gene therapy, and immunotherapy.

The methods of treating or preventing cancer comprise obtaining an isolated or purified immunogen polypeptide. Immunogen polypeptides suitable for the methods of treating or preventing cancer are described above. Such immunogen polypeptides may comprise, consist essentially of, or consist of a) all or portions of the major CSA binding channel of VAR2CSA; b) all or portions of the minor CSA binding channel of VAR2CSA; and/or c) all or portions of the arm segment of VAR2CSA. In aspects the immunogen polypeptide comprises, consists essentially of, or consists of all or portions of the major CSA binding channel. In aspects the immunogen polypeptide comprises, consists essentially of, or consists of all or portions of the minor CSA binding channel. In aspects the immunogen polypeptide comprises, consists essentially of, or consists of all or portions of the arm segment. The immunogen polypeptide may comprise one or more conserved VAR2CSA binding channel residues as described above. In certain aspects, the conserved VAR2CSA binding channel residues are located in the major CSA binding channel of VAR2CSA.

In aspects, the method of treating or preventing cancer in a subject comprises, consists essentially of, or consists of obtaining an immunogen polypeptide having at least 80% identity with any one of SEQ ID NOs: 3-54. In aspects, the immunogen polypeptide comprises, consists essentially of, or consists of a polypeptide having at least 85% identity with any one of SEQ ID NOs: 3-54. In aspects, the immunogen polypeptide comprises, consists essentially of, or consists of a polypeptide having at least 90% identity with any one of SEQ ID NOs: 3-54. In aspects, the immunogen polypeptide comprises, consists essentially of, or consists of a polypeptide having at least 95% identity with any one of SEQ ID NOs: 3-54. In some aspects the immunogen polypeptide comprises, consists essentially of, or consists of any one of SEQ ID Nos: 3-54.

In aspects, the method of treating or preventing cancer in a subject comprises, consists essentially of, or consists of administering an effective amount of a pharmaceutical composition comprising the immunogen polypeptide. Examples of suitable pharmaceutical formulations are provided above. For the purposes of cancer treatment, compositions can be administered in dosages and by techniques well known to those skilled in the medical or veterinary arts, taking into consideration such factors as the age, sex, weight, species and condition of the recipient subject, and the route of administration.

In the context of methods of treating or preventing a disease (e.g. cancer), “therapeutically effective amount” refers to an amount that relieves (to some extent, as judged by a skilled medical practitioner) one or more symptoms of the disease or condition in a mammal, e.g. the symptoms of cancer. Additionally, by “therapeutically effective amount” is meant an amount that returns to normal, either partially or completely, physiological or biochemical parameters associated with or causative of a disease or condition. A clinician skilled in the art can determine the therapeutically effective amount of a composition in order to treat or prevent a particular disease condition. The precise amount of the composition required to be therapeutically effective will depend upon numerous factors, e. g., such as the specific activity of the active agent, the delivery device employed, physical characteristics of the agent, purpose for the administration, in addition to many patient specific considerations. The determination of amount of a composition that must be administered to be therapeutically effective is routine in the art and within the skill of an ordinarily skilled clinician.

The route of administration of the pharmaceutical composition in accordance with the methods of treating or preventing disease may be percutaneous, via mucosal administration (e.g., oral, nasal, anal, vaginal) or via a parenteral route (intradermal, intramuscular, subcutaneous, intravenous, or intraperitoneal). Compositions can be administered alone, or can be co-administered or sequentially administered with other treatments or therapies.

In aspects, forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray or mixed in food and/or water or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired.

In aspects the composition used in the methods of treatment includes an additional therapeutic agent. Suitable additional therapeutic agents are described above. In aspects the additional therapeutic agent is an anti-cancer agent. In aspects, the additional therapeutic agent is conjugated or coupled to the immunogen polypeptide as described above. Accordingly, the disclosure also provides a method for delivering the additional therapeutic agent to the site of a disease in the subject. The disease site may be, for example, a tumor. In such aspects, the site of disease may be a tumor that expresses CSA.

In aspects of the disclosure, the methods of treatment can consist essentially of the methods of treatment as described herein, such that any variation does not materially change the characteristics of the method. In other aspects of the disclosure, the methods of treatment consist of the methods of treatment as described herein.

### Nucleic Acids

Aspects of the present disclosure include nucleic acids comprising regions encoding the disclosed immunogen polypeptides. Such nucleic acids may comprise DNA or RNA. In aspects, the nucleic acids may be isolated or purified. In other aspects the nucleic acids may not be isolated or purified. In aspects, the nucleic acid encodes an immunogen polypeptide having at least 80% identity with any one of SEQ ID NOs: 3-54. In other aspects, the nucleic acid may encode and immunogen polypeptide comprising any one of SEQ ID NOs: 3-54.

Aspects of the disclosure comprise a biological vector comprising nucleic acids encoding the immunogen polypeptide.

In aspects of the disclosure, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises, consists essentially of, or consists of a nucleotide sequence encoding the mRNA, protein, polypeptide, or peptide, and the vector is contacted with the cell under conditions sufficient to have the mRNA, protein, polypeptide, or peptide expressed within the cell. The vectors of the disclosure are not naturally-occurring as a whole. However, parts of the vectors can be naturally-occurring. The disclosed recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vectors of the disclosure can be prepared using standard recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the disclosed expression vectors include, for instance, neomycin/G418 resistance genes, hygromycin resistance genes, histidinol resistance genes, tetracycline resistance genes, and ampicillin resistance genes.

The vector may further comprise regulatory sequences which are operably linked to the nucleotide sequence encoding the protein constructs which permits one or more of the transcription, translation, and expression protein constructs in a cell transfected with the vector or infected with a virus that comprises, consists essentially of, or consists of the vector. As used herein, “operably linked” sequences include both regulatory sequences that are contiguous with the nucleotide sequence encoding the protein construct and regulatory sequences that act in trans or at a distance to control the nucleotide sequence encoding the protein construct.

The regulatory sequences may include appropriate transcription initiation, termination, promoter and enhancer sequences; RNA processing signals such as splicing and polyadenylation (polyA) signal sequences; sequences that stabilize cytoplasmic mRNA; sequences that enhance translation efficiency (i.e., Kozak consensus sequence); sequences that enhance protein stability.

In aspects, the biological vector comprises, consists essentially of, or consists of a promotor that drives expression of the protein construct. The promoter may be any promoter suitable for expressing the protein construct in a target cell, e.g., a mammalian cell. The promoter may be inducible or constitutive. In aspects of the disclosure, the promoter is suitable for expressing the protein construct in a particular cell type. In this regard, the promoter may be cell-specific.

In some aspects the vector is a pcDNA3.1 vector.

In some aspects the vector is a viral vector. Examples of suitable viral vectors include retroviral vectors, lentiviral vectors, adenoviral vectors, adeno-associated viral (AAV) vectors.

In aspects of the disclosure, a nucleic acid with regions encoding the disclosed immunogen polypeptides may be formulated into a pharmaceutical composition. In aspects, such formulations may comprise a pharmaceutically acceptable carrier. Aspects of the disclosure include nucleic acid vaccine formulations comprising plasmid DNA, viral vectors or messenger RNA (mRNA) or modified messenger RNA (mmRNA) encoding the disclosed immunogen polypeptides.

Certain aspects of the disclosure include mRNA or mmRNA vaccine formulations wherein the mRNA or mmRNA contains a translatable region encoding the disclosed immunogen polypeptides. mmRNA may be modified in any suitable manner. Such modifications may include chemical modifications such as, for example, the inclusion of the modified nucleoside 1-methyl-pseudouridine. The modifications in mmRNA may function, e.g., to provide for increased polypeptide production and substantially reduced innate immune response in the cell, as compared to a composition comprising a corresponding unmodified mRNA. Methods for preparing and using mRNA, mmRNA and similar vaccine formulations are known in the art, and are described in, for example, U.S. Pat. No. 10,898,574, which is incorporated by reference in its entirety.

Aspects of the disclosure also include methods for producing the disclosed immunogen polypeptides in a cell in a subject, comprising administering to the subject a pharmaceutical composition comprising a modified messenger RNA (mmRNA) such that the mmRNA is introduced into the cell, wherein the mmRNA comprises a translatable region encoding the immunogen polypeptide.

Aspects of the disclosure also include methods of vaccinating subjects comprising administering a vaccine formulation comprising a nucleic acid with regions encoding the disclosed immunogen polypeptides, e.g., an mRNA or mmRNA vaccine formulation.

In aspects of the disclosure, the nucleic acids can consist essentially of the nucleic acids as described herein, such that any variation does not materially change the characteristics of the nucleic acids. In other aspects of the disclosure, the nucleic acids consist of the nucleic acids as described herein.

The following are aspects of the present disclosure:
1. An immunogen polypeptide comprising:
a) all or a portion of the major CSA binding channels of VAR2CSA;
b) all or a portion of the minor CSA binding channels of VAR2CSA;
c) all or a portion of the arm segment of VAR2CSA; or
d) combinations thereof.
2. The immunogen polypeptide of aspect 1, wherein the immunogen polypeptide comprises all or a portion of each of the NTS, DBL1X, DBL2X and DBL4ε regions of VAR2CSA.
3. The immunogen polypeptide of aspects 1 or 2, wherein the VAR2CSA fragment comprises all or a portion of ID1.
4. The immunogen polypeptide of any one of aspects 1-3, wherein the immunogen polypeptide comprises all or a portion of each of the ID2a and ID2b regions of VAR2CSA.
5. The immunogen polypeptide of any one of aspect 1-4, wherein the immunogen polypeptide comprises all or a portion of each of the DBL3X region of VAR2CSA.
6. The immunogen polypeptide of any one of aspects 1-5, wherein the immunogen polypeptide comprises all or a portion of the ID3 region of VAR2CSA.
7. The immunogen polypeptide of aspect 1, wherein the immunogen polypeptide comprises one or more conserved CSA binding channel residues.
8. The immunogen polypeptide of aspect 7, wherein the conserved CSA binding channel residues are located in the major CSA binding channels of VAR2CSA.
9. The immunogen polypeptide of aspect 1, wherein the immunogen polypeptide comprises one or more amino acids corresponding to amino acids 44-46, 47-49, 556-558, 560-563, 575-577, 821-823, 827-836, 911-913, 951-953, 967-971, 1784-1786, 1879-1881, or 1888-1890 of SEQ ID NO: 1.
10. The immunogen polypeptide of aspect 9, wherein the immunogen polypeptide comprises one or more amino acids corresponding to amino acids 45, 48, 557, 561, 562, 576, 822, 828, 829, 832, 835, 912, 952, 968, 970, 1785, 1880, 1889 of SEQ ID NO: 1.
11. The immunogen polypeptide of aspect 1, wherein the immunogen polypeptide has at least 80% identity with any one of SEQ ID NOs: 3-54, and a pharmaceutically acceptable carrier.
12. The immunogen polypeptide of aspect 11, wherein the immunogen polypeptide comprises any one of SEQ ID Nos: 3-54.
13. The immunogen polypeptide of any one of aspects 1-12, wherein the immunogen polypeptide is isolated or purified.
14. A pharmaceutical composition comprising one or more immunogen polypeptides according to any one of aspects 1-13, and a pharmaceutically acceptable carrier.
15. The pharmaceutical composition of aspect 14, wherein the composition comprises multimers of the one or more immunogen polypeptides.
16. The pharmaceutical composition of aspect 14 or 15, wherein the one or more immunogen polypeptides are in nanoparticle form.
17. The pharmaceutical composition of any one of aspects 14-16, further comprising one or more additional therapeutic agents.
18. The pharmaceutical composition of aspect 17, wherein the one or more additional therapeutic agents are coupled to the one or more immunogen polypeptides.
19. The pharmaceutical composition of aspect 17 or 18, wherein the one or more additional therapeutic agents comprise an anti-cancer agent.
20. The pharmaceutical composition of any one of aspects 14-19, wherein the composition is suitable for parenteral administration to a subject.
21. A nucleic acid encoding one or more immunogen polypeptides according to any one of aspects 1-11, wherein the nucleic acid is isolated or purified.
22. The nucleic acid of aspect 21, wherein the immunogen polypeptide has at least 80% identity with any one of SEQ ID NOs: 3-54.
23. The nucleic acid of aspect 22, wherein the immunogen polypeptide comprises any one of SEQ ID NOs: 3-54.
24. A vector comprising the nucleic acid of any one of aspects 21-23.
25. A pharmaceutical composition comprising one or more nucleic acids according to any one of aspects 21-23, and a pharmaceutically acceptable carrier.
26. The pharmaceutical composition of aspect 25, wherein the composition is suitable for parenteral administration to a subject.
27. A composition for use in vaccinating a subject against malaria, the composition comprising:
a) one or more immunogen polypeptides according to any one of aspects 1-13, or one or more nucleic acids according to any one of aspects 21-23; and
b) a pharmaceutically acceptable carrier;
   wherein the pharmaceutical composition is administered to the subject.
28. The composition for use of aspect 27, wherein the malaria is placental malaria.
29. The composition for use of aspect 27 or 28, wherein the subject is a mammal.
30. The composition for use of aspect 29, wherein the mammal is a human.
31. The composition for use of any one of aspects 27-30 wherein the pharmaceutical composition is administered to the subject parenterally.
32. The composition for use of any one of aspects 27-31, wherein the administration results in an immune response in the subject, and wherein the immune response in the subject is sufficient to reduce risk of malaria infection.
33. The composition for use of aspect 32, wherein the immune response in the subject is strain transcending.
34. A composition for use in treating or preventing cancer in a subject, the composition comprising:
a) an immunogen polypeptide according to any one of aspects 1-13; and
b) a pharmaceutically acceptable carrier;
   wherein the pharmaceutical composition is administered to the subject.
35. The composition for use of aspect 34, wherein the subject is a mammal.
36. The composition for use of aspect 35, wherein the mammal is a human.
37. The composition for use of any one of aspects 34-36, wherein the pharmaceutical composition further comprises one or more additional therapeutic agents.
38. The composition for use of aspect 37, wherein the one or more additional therapeutic agents are coupled to the immunogen polypeptide.
39. The composition for use of aspect 37 or 38, wherein the one or more additional therapeutic agents comprises an anti-cancer agent.
40. The composition for use of any one of aspects 34-39, wherein the pharmaceutical composition is administered to the subject parenterally.
41. The composition for use of any one of aspects 34-40, wherein the cancer is a CSA expressing cancer.
42. The composition for use of aspect 41, wherein the CSA expressing cancer is selected from colorectal cancer, lung cancer, breast cancer, prostate cancer, osteosarcoma, melanoma, diffuse gliomas and brain cancer.
43. A method of producing an immunogen polypeptide of any one of aspects 1-13 in a cell in a subject in need thereof, comprising administering to the subject a pharmaceutical composition comprising a modified messenger RNA (mmRNA) such that the mmRNA is introduced into the cell, wherein the mmRNA comprises a translatable region encoding the immunogen polypeptide, and wherein the immunogen polypeptide is produced in the cell.

The following examples further illustrate the invention but, of course, should not be construed as in any way limiting its scope.

### EXAMPLE 1

This example describes a study identifying three dimensional structural characteristics of VAR2C SA and associated analysis.

The following methods were used in this Example:

### Purification of VAR2CSA NF54 and FCR3 in Expi293 Cells

The wild type VAR2CSA NF54 and VAR2CSA FCR3 were expressed in Expi293 (Thermo Fisher) cells according to the manufacturer's protocols. In brief, the cells were grown shaking at 37° C. and 8% CO2, maintaining cultures at continuous log phase growth (3.0-5×10⁶) for 3-4 passages after thawing. The day before transfection, 500 mL of culture was seeded at a density of 2.5-3×10⁶ cells/mL in a 2 L flask. The day of transfection, cells were diluted back to 2.5-3×10⁶ prior to transfection. The plasmid DNA was diluted with 25 mL of Opti-MEM I medium (Thermo Fisher) to a final concentration of 1 μg/mL.

Then, 1.4 ml ExpiFectamin™ 293 Reagent (Thermo Fisher) was diluted with 25 ml Opti-MEM I medium, gently mixed and incubated at room temperature for 5 minutes. The diluted ExpiFectamine™ 293 Reagent was then added to the diluted plasmid DNA, mixed by swirling, and incubated at room temperature for 20 minutes. The mixture was added to the cells slowly while swirling the flask. The flask was returned to the incubator at 37° C. and 8% CO2. After 20 hours of incubation, ExpiFectamine™ 293 Transfection Enhancer 1 (Thermo Fisher) and ExpiFectamine™ 293 Transfection Enhancer 2 (Thermo Fisher) were added to the transfection flask.

### Purification of VAR2CSA NF54 and FCR3

The cultures were centrifuged at 5000 rpm for 15 min five days post-transfection. The supernatant was collected and loaded onto Ni Sepharose® Excel columns (GE Healthcare), which were manually packed in a glass gravity column. The column was washed twice with 10 column volumes of wash buffer (25 mM HEPES, pH 7.4, 150 mM NaCl, 25 mM imidazole) and eluted with 5 column volumes of elution buffer (25 mM Tris-HCl, pH 7.4, 150 mM NaCl, 250 mM imidazole). The elutes were concentrated with a 100 kDa cutoff centrifugal filter unit (Millipore Sigma) to 1 ml and further purified by size-exclusion chromatography (Superose 6 Increase 10/300, GE Healthcare) in buffer A (10 mM HEPES, pH 7.4, 100 mM NaCl ). The peak fractions were collected and verified by SDS-PAGE before EM grids preparation.

### On-Column Crosslinking of VAR2CSA FCR3

In order to mildly stabilize the protein, on-column crosslinking of VAR2CSA FCR3 was performed as described. First, a bolus of glutaraldehyde (200 μl 0.25% v/v) was injected to a pre-equilibrated Superose 6 Increase 10/300 column in buffer A and run at 0.25 ml/min for 16 min (a total of 4 ml buffer). Then, the column flow was paused, and the injection loop was flushed using buffer followed by injection of purified VAR2CSA FCR3 (200 μl volume, at 3 μM concentration). Subsequently, the column was run at 0.25 ml/min and 0.3 ml fractions were collected for EM grids preparation.

### VAR2CSA-CSA Complex Reconstruction

The Chondroitin sulfate A sodium salt from bovine trachea (Sigma) was dissolved in buffer A to 10 mg/ml. Then VAR2CSA was mixed with CSA at a molar ratio of 1:4. The mixture was incubated on ice for 30 min before EM grids preparation.

### Cryo-EM Grid Preparation and Data Collection

The homogeneity of samples was first assessed by negative-stain EM with 0.7% (w/v) uranyl formate or 1% uranyl acetate as described. Before preparing grids for Cryo-EM, the freshly purified protein sample was centrifuged at 13,000 g for 2 min to remove potential protein aggregates, and the protein concentration was measured with a NanoDrop spectrophotometer (Thermos Fisher Scientific). The final protein concentration used for Cryo-EM grid preparation is 0.8 mg/ml.

The protein sample was kept on ice before grid preparation. A 3.5 μL aliquot of protein was applied to a glow-discharged Quantifoil 300 mesh 1.2/1.3 carbon grid (Quantifoil) that had been glow-discharged for 90 s at 10 mA with PELCO easiGlow Glow Discharge Set. VAR2CSA FCR3 and the VAR2CSA FCR3 cross-linked samples were blotted for 3 s and VAR2CSA NF54 CSA complex was blotted for 2 s with a blot force of 3 and 55/20 mm filter paper (TED PELLA) before plunged into liquid ethane with a Vitrobot Mark VI (FEI) set at 16° C. and 100% humidity. After screening multiple grids, three grids made with the samples VAR2CSA NF54 in complex with CSA, VAR2CSA FCR3 alone and the cross-linked VAR2CSA FCR3 were chosen for data collection based on the evaluation of data quality.

The NF54+CSA and FCR3 dataset were collected on the 300 keV Titan Krios with Gatan BioQuantum Image Filter in NIH National Cancer Institute (NCI)/NICE facility. The images were recorded with a 20 eV slit post-GIF K2 Summit camera in super-resolution counting mode at a nominal magnification of 130,000× and a defocus range from −0.7 to −2.0 μm. Exposures of 8 s were dose-fractionated into 40 frames (200 ms per frame), with an exposure rate of 8 electrons·pixel−1·s−1, resulting in a total exposure of 57 electrons·A−2, The data collection was automated using the SerialEM software package.

The FCR3 crosslink dataset was collected on Titan Krios electron microscopes in NIH Multi-Institute Cryo-EM Facility (MICEF). The images were recorded with a K2 Summit camera equipped with a Gatan Quantum LS imaging energy filter with the slit width set at 20 eV in counting mode at a nominal magnification of 130,000× and a defocus range from −1.0 to −2.0 μm. Exposures of lOs were dose-fractionated into 50 frames (200 ms per frame), with an exposure rate of 71.2 electrons·Å−2, The data collection was automated using the Leginon software package.

### Image Processing

6,196 dose-fractionated movies of VAR2CSA NF54+CSA were collected. The processing was done within cryoSPARC (v2.14.2). Motion correction was done by cryoSPARC's Patch motion correction with an output F-crop factor of ½. CTF estimation for each micrograph was calculated with Patch CTF estimation. Particles were autopicked from each micrograph with the blob picker from cryoSPARC and then sorted by 2D classification for two rounds to exclude bad particles. 858,299 particles were selected. The particles were used to generate an ab initio map in cryoSPARC. Particles were classified into 5 classes using the low-pass-filtered (30 Å) ab initio map as a template. Class 1 and Class 4 with a total of 299,571 particles, which has a clear map of the core region, was selected to conduct NU-refinement and generated a 3.5 Å map. A mask covering core regions were then used to perform local refinement and generated a 3.36 Å map. The map of the core is local filtered with a b-factor of −76.4 in FIG. 1c. Class 1 which has a clear density of the whole protein were selected solely with 157,702 particles to perform NU-Refinement and generated a 3.87 Å map of the full length complex. A mask covering DBL5ε and 6ε regions were then used to perform local refinement and generated a 4.88 Å map.

100,108 dose-fractionated movies of VAR2CSA FCR3 were collected on a 300-kV Titan Krios (FEI) equipped with a K2 Summit direct electron detector (Gatan). Similarly, the processing is done within cryoSPARC (v2.14.2). Motion correction was done by cryoSPARC's Patch motion correction with an output F-crop factor of ½. CTF estimation for each micrograph was calculated with Gctf (v1.06, https://www2.mrc-lmb.cam.ac.uk/research/locally-developed-software/zhang-software/). Particles were auto picked from each micrograph with the blob picker from cryoSPARC and then sorted by 2D classification for two rounds to exclude bad particles. 783,088 particles were selected. The dataset contained 783,088 particles. The particles were used to generate an ab initio map in cryoSPARC. Particles were classified into 10 classes using the low-pass-filtered (30 Å) ab initio map as a template. Class 4 with a total of 271,442 particles was selected to conduct NU-refinement and generated a 4 Å map. A mask covering DBL5ε and DBL6ε domains were then used to perform local refinement and generated a 4.69 Å map

4,739 dose-fractionated movies of VAR2CSA FCR3 were collected. The processing was also done within cryoSPARC. Full frame motion correction was done by cryoSPARC's own implementation. CTF estimation for each micrograph was calculated with Gctf. 2,010,465 articles were auto picked from each micrograph with the blob picker from cryoSPARC and then sorted by 2D classification for two rounds to exclude bad particles. 505,409 particles were selected. The particles were used to generate an ab initio map in cryoSPARC. Particles were classified into 3 classes using the low-pass-filtered (30 Å) ab initio map as a template. Class 1 with a total of 319,520 particles was selected to conduct NU-refinement and generated a 3.52 Å map. A mask covering the core regions were then used to perform local refinement and generated a 3.38 Å map.

### Model Building and Refinement

First the model was built for the core of VAR2CSA FCR3 cross-linked. The crystal structures of DBL3X+4ε (PDB ID 4P1T) was used as a starting model and was fitted and refined into the Cryo-EM density map with PHENIX (v1.18.2) ‘Dock in map’ and ‘Real-space refinement. The successful docking and the clear fitting of the DBL3X+4 side chains with the density indicated the fitting was correct. The clear density of a α-helix (ID3) that connects the C-terminus of DBL4ε with the flexible arm which has a density of two tandem DBL domains helped us confirm the core is made up of DBL1X to DBL4ε while the arm is consists of DBL5ε and 6ε. The structures of DBL1X, DBL2X and ID2b were predicted from Phyre2 (http://www.sbg.bio.ic.ac.uk/˜phyre2/html/page.cgi?id=index) and then fitted and refined into the map by PHENIX (v1.18.2). The missing regions were manually built in COOT (v0.9). The atomic model for the core was refined using phenix.real_space_refine global minimization (default), morphing and simulated annealing rama potential.

The model of the VAR2CSA crosslink core was used to build the VAR2CSA FCR3 structure by docking the model into the VAR2CSA FCR3 map and auto refined by PHENIX. To build the arm region of VAR2CSA, the crystal structure of DBL6ε (PDB ID 2Y8D) and a predicted DBL5ε structure with Phyre2 was used. The structures were fit in the Cryo-EM density map from local refinement with Chimera using the “fit in map” tool. The atomic model for the arm was refined using phenix.real_space_refine global minimization (default), morphing and simulated annealing rama potential.

The model of core and arm region of CSA-VAR2CSA NF54 complex was built separately by fitting the corresponding model of VAR2CSA FCR3 into the map and manually mutating the residues and fragments adjustment. The CSA model was built with the C4S tetrasaccharide from the structure of the Shh-Chondroitin-4-Sulfate (C4S) Complex (PDB ID 4C4M). The atomic models was refined using phenix.real_space_refine global minimization (default), morphing and simulated annealing rama potential. The models of the core and arm were combined by fitting both of the maps together.

Structural and map figures were prepared in Chimera (v1.13.1, https://www.cgl.ucsf.edu/chimera/), ChimeraX (v1.0, https://www.rbvi.ucsf.edu/chimerax/) which are developed by UCSF, and PyMOL (v2.1, https://pymol.org /2/).

### Overall Structure of the CSA-VAR2CSA Complex

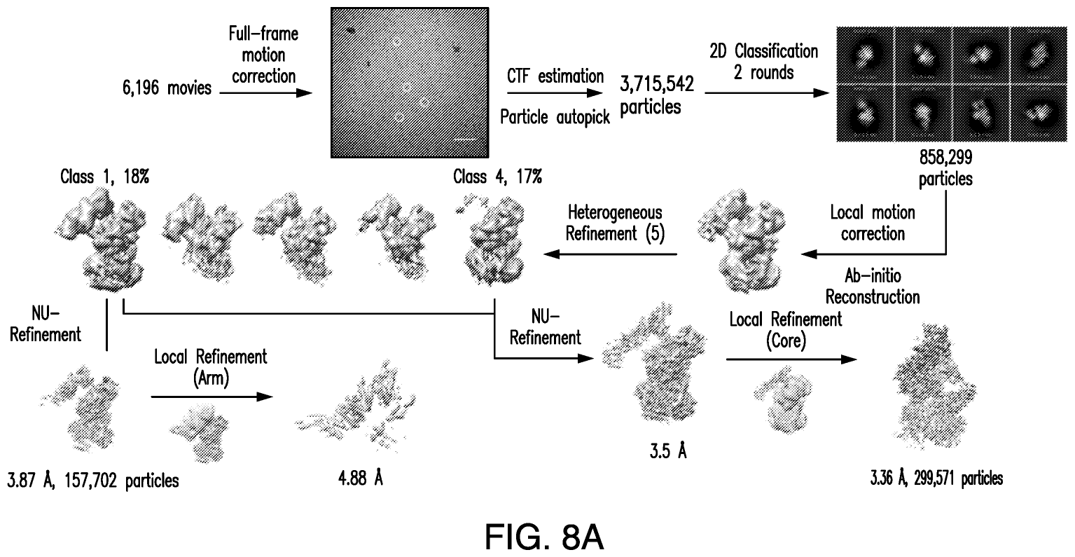

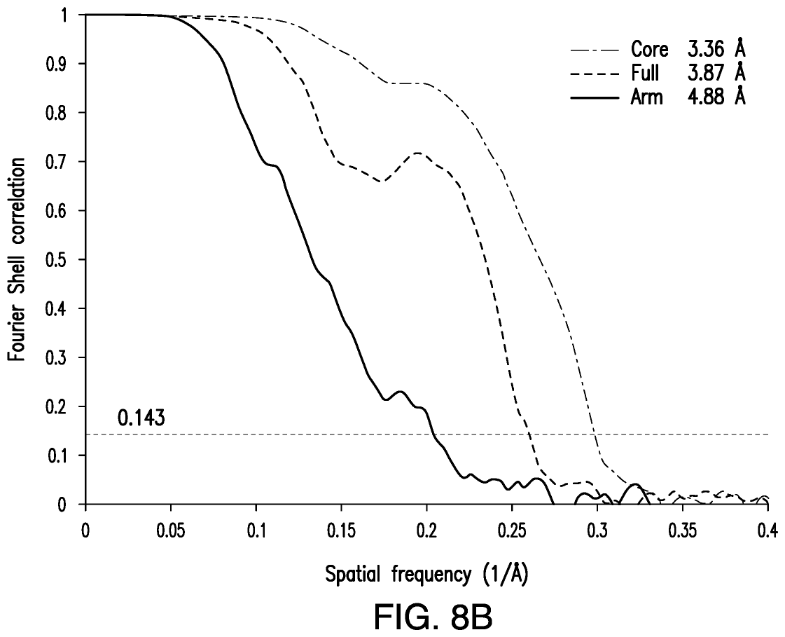

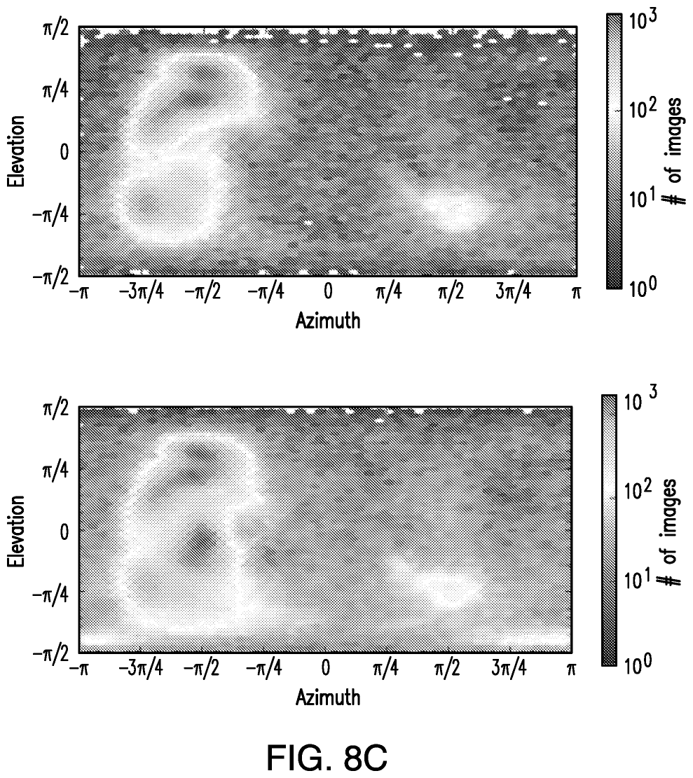

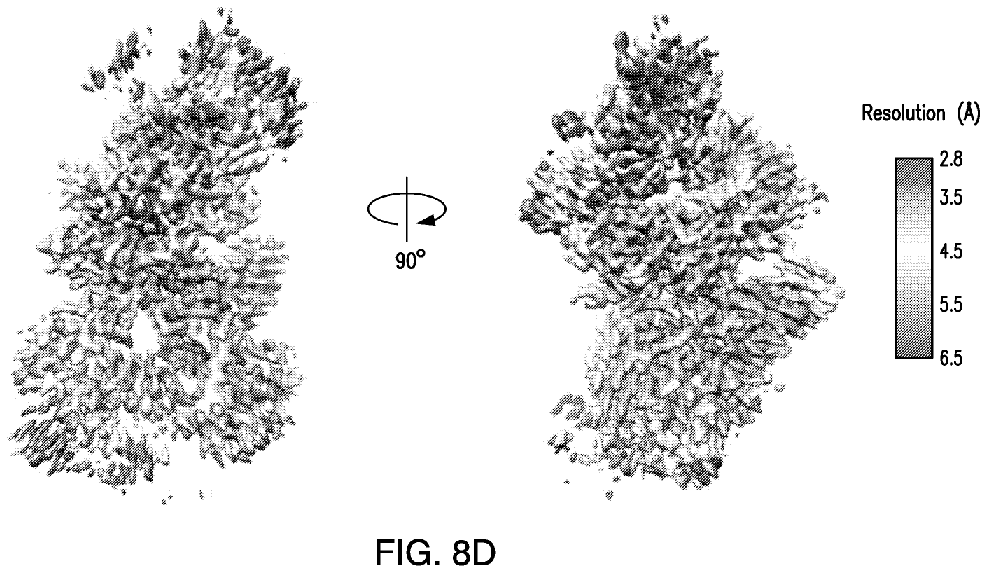

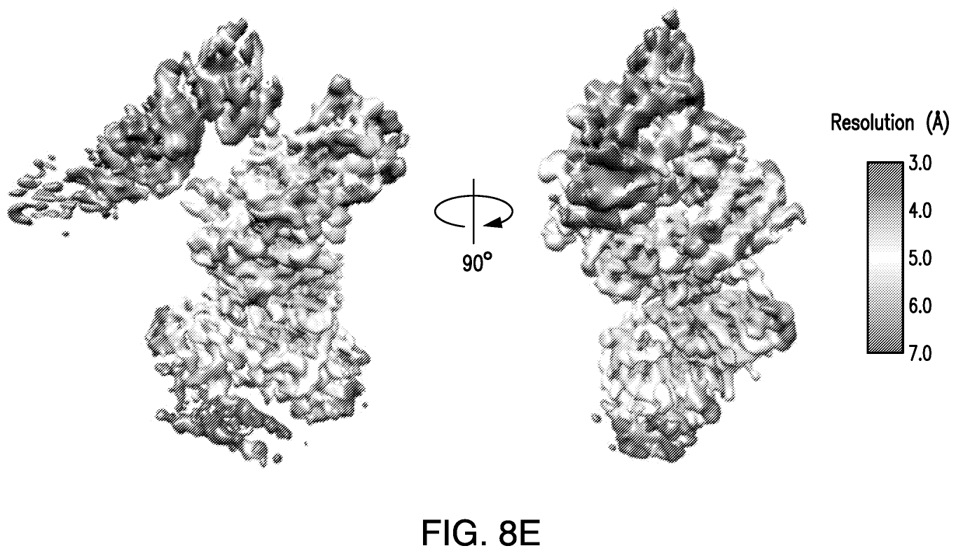

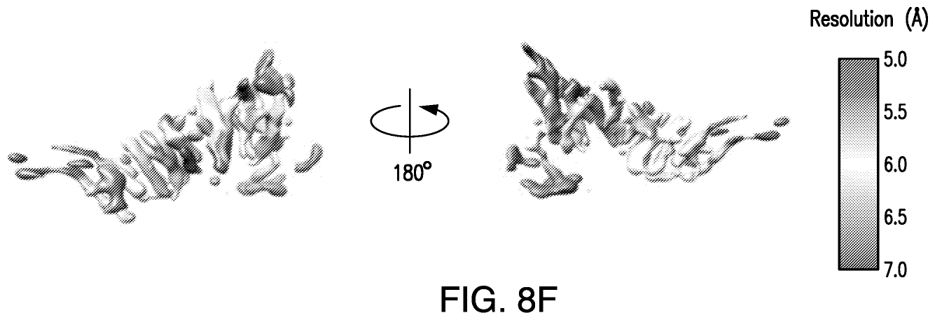

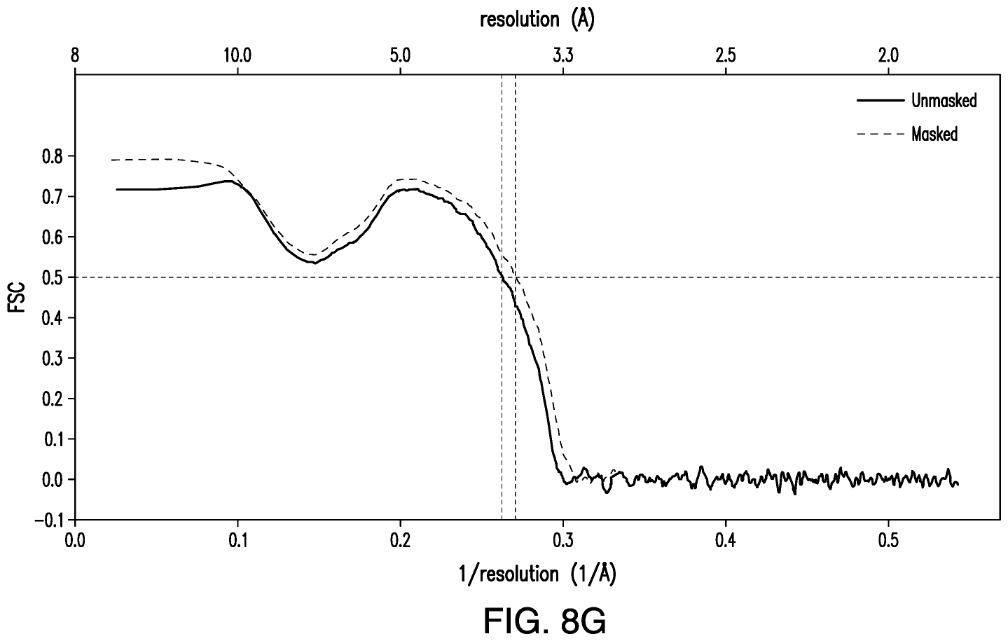

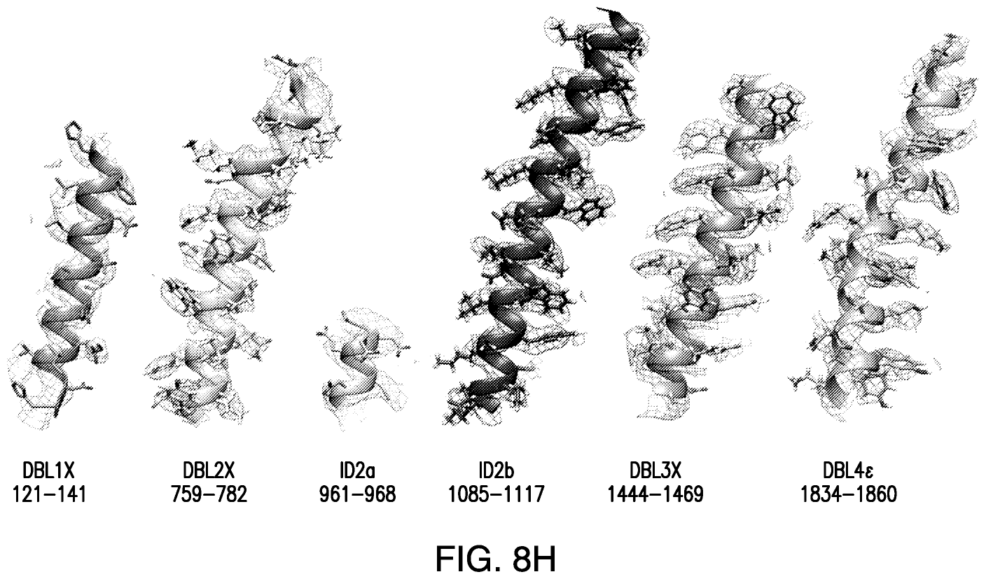

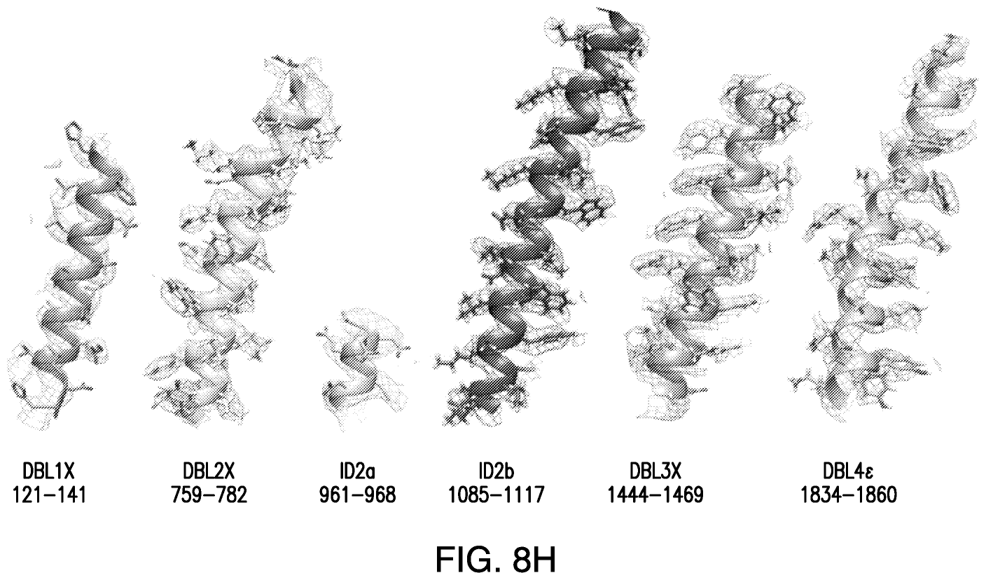

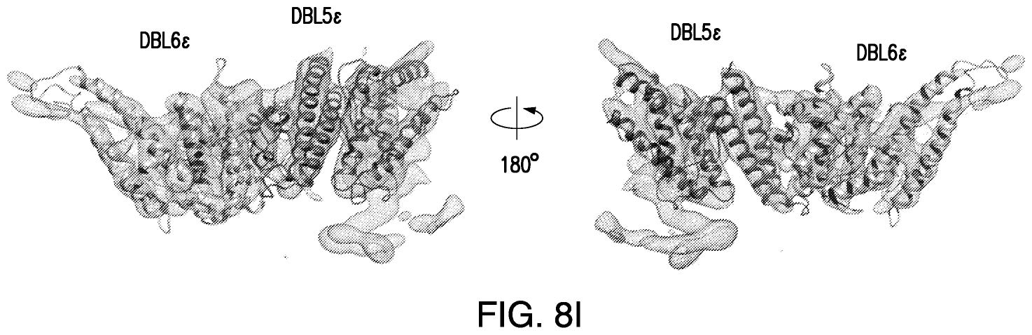

VAR2CSA was expressed from parasite strain NF54 (VAR2CSA NF54) in Expi293 cells and purified for the Cryo-EM study of VAR2CSA in complex with CSA 6,196 movies were collected allowing for a 3.82 Å reconstruction of VAR2CSA NF54 in complex with CSA (FIG. 8a). VAR2CSA NF54 exhibits an architecture comprised of a stable core and a flexible arm (FIG. 1a-c). Local refinement of the core improved the resolution to 3.36 Å, and local refinement of the arm resulted in a 4.88 Å map (FIG. 8a-f). A CSA dodecamer spans the core domain and binds in a channel termed the major binding channel (FIG. 1b-d). Another potential binding site for CSA was observed in a second channel termed the minor binding channel with weak density that could be modeled as a CSA monosaccharide (FIG. 1b, d). This binding of CSA polymer within channels of VAR2CSA is reminiscent of the binding model proposed for EBA-175 binding to glycophorin A during P. falciparum invasion of erythrocytes, where the glycophorin A receptor feeds through channels created by EBA-17530. EBA-175 is a protein related to VAR2CSA that belongs to the erythrocyte binding-like (EBL) family involved in the recognition of sialic acid on erythrocyte glycoproteins during erythrocyte invasion by P. falciparum. The inventor's final model for VAR2CSA NF54 spans residues 32 to 2607 of VAR2CSA NF54 with a few flexible loops and ID1 omitted as these segments were not ordered in the reconstruction (FIG. 1c, 8g-I). Table 2 provides Cryo-EM data.

TABLE 3
Cryo-EM data statistics.

| | VAR2CSA | | | | |
|---|---|---|---|---|---|
| | FCR3 | VAR2CSA | VAR2CSA | VAR2CSA | VAR2CSA |
| | (Crosslink) | FCR3 | FCR3 | NF54 + CSA | NF54 + CSA |
| | Core | Core | Arm | Core | Arm |
| Data collection and processing | | | | | |
| Magnification | 130,000 | 130,000 | 130,000 | 130,000 | 130,000 |
| Voltage(kV) | 300 | 300 | 300 | 300 | 300 |
| Electron exposure (e−/Å²) | 71.2 | 57 | 57 | 57 | 57 |
| Defocus range (μm) | −1.0 to −2.0 | −0.7 to −2.0 | −0.7 to −2.0 | −0.7 to −2.0 | −0.7 to −2.0 |
| Pixel size (Å) | 1.06 | 1.06 | 1.06 | 1.06 | 1.06 |
| Symmetry imposed | C1 | C1 | C1 | C1 | C1 |
| Initial particle images (no.) | 2,010,465 | 4,583,211 | 4,583,211 | 3,715,542 | 3,715,542 |
| Final particle images (no.) | 319,520 | 271,442 | 271,442 | 299,571 | 157,702 |
| Map resolution (Å) | | | | | |
| FSC threshold | 0.143 | 0.143 | 0.143 | 0.143 | 0.143 |
| Map resolution range (Å) | 3.38 | 4 | 4.69 | 3.36 | 4.88 |
| Refinement | | | | | |
| Initial model used (PDB code) | 4PIT | | 2Y8D | 4C4M | |
| Model resolution (Å) | 3.4 | 4.0 | 6.5 | 3.4 | 7.2 |
| 0.143 FSC threshold | | | | | |
| Map sharpening B factor (Å2) | −40.32 | −129.03 | −159.35 | −64.44 | −209.55 |
| Model composition | | | | | |
| Nonhydrogen atoms | 20361 | 20364 | 4521 | 19647 | 4357 |
| Protein residues | 1441 | 1441 | 537 | 1424 | 523 |
| Ligands | — | — | — | | |
| B factors (Å2) | | | | | |
| Protein | 65.07 | 26.17 | 46.87 | 22.33 | 462.86 |
| Ligand | — | — | — | 2.62 | |
| R.M.S. deviations | | | | | |
| Bond lengths (Å) | 0.001 | 0.001 | 0.003 | 0.001 | 0.002 |
| Bond angles (°) | 0.365 | 0.367 | 0.631 | 0.344 | 0.559 |
| Validation | | | | | |
| MolProbity score | 1.83 | 1.89 | 1.69 | 1.94 | 1.87 |
| Clashscore | 10.46 | 13.47 | 6.68 | 13.07 | 12.25 |
| Poor rotamers (%) | 0.54 | 0.00 | 0.00 | 0.54 | 0 |
| Ramachandran plot | | | | | |
| Favored (%) | 95.81 | 96.25 | 95.45 | 95.53 | 96.07 |
| Allowed (%) | 4.19 | 3.75 | 4.55 | 4.47 | 3.93 |
| Disallowed (%) | | | | | |

### An Interwoven Domain Architecture Stabilizes VAR2CSA

VAR2CSA is primarily composed of α-helices and extensive loops that adopt an overall shape resembling the number 7 (FIG. 1c). CryoSPARC 3D variability analysis confirms that the region composed of DBL2X to ID3 forms a relatively stable core, while DBL5ε-DBL6ε forms a flexible arm and DBL1X exhibits some structural flexibility. The 6 individual DBL domains of VAR2CSA adopt the classical DBL domain fold, consisting of an α-helical core decorated by extensive loops (FIG. 2a). The individual domains interact in an interwoven manner to stabilize the compact tertiary structure (FIG. 1c, d). DBL4ε, the most conserved DBL domain of the six, is located at the center of VAR2CSA and unites the whole structure by directly interacting with all the other domains except DBL1X and DBL5ε (FIG. 1c, d). DBL1X and DBL5ε are connected to DBL4ε via the NTS and ID3, respectively (FIG. 1c, d). The NTS (residue 32-49) is a twisted loop surrounding DBL1X and serves as the mortar holding together DBL1X and DBL4ε, with high conservation among diverse VAR2CSA strains (FIG. 2a-c). ID3 is a long helix that closely interacts with ID2 and connects DBL5ε with the core (FIG. 2a, d). A total of 31 pairs of disulfide bridges were identified in the final model (FIG. 2e and Table 3).

TABLE 4
Intra- and Inter - Domain disulfide bonds
Intra-domain disulfide bonds

| | |
|---|---|
| DBL1X | C51-C227, C66-C105, C151-C258 |
| DBL2X | C643-C745, C769-C901, C783-C801, C805-C955, C797- |
| | C957 |
| ID2a | C978-C996 |
| ID2b | C1088-C1193, C1140-C1171 |
| DBL3X | C1210-C1411, C1221-C1264, C1242-C1255, C1339- |
| | C1431, C1456-C1528, C1470-C1483, C1487-C1556 |
| DBL4ε | C1578-C1614, C1670-C1777, C1671-C1901, C1802- |
| | C1904, C1816-C1833, C1891-C1902 |
| ID3 | |
| DBL5ε | C2202-C2218 |
| DBL6ε | C2334-C2480, C2520-C2604, C2534-C2551, C2598-C2602 |

Inter-domain disulfide bonds

| | |
|---|---|
| ID2a-ID2b | C966-C1090 |
| DBL4X-ID3 | C1837-C1927 |

### Structural Conservation Within the EBL and PfEMP1 Families

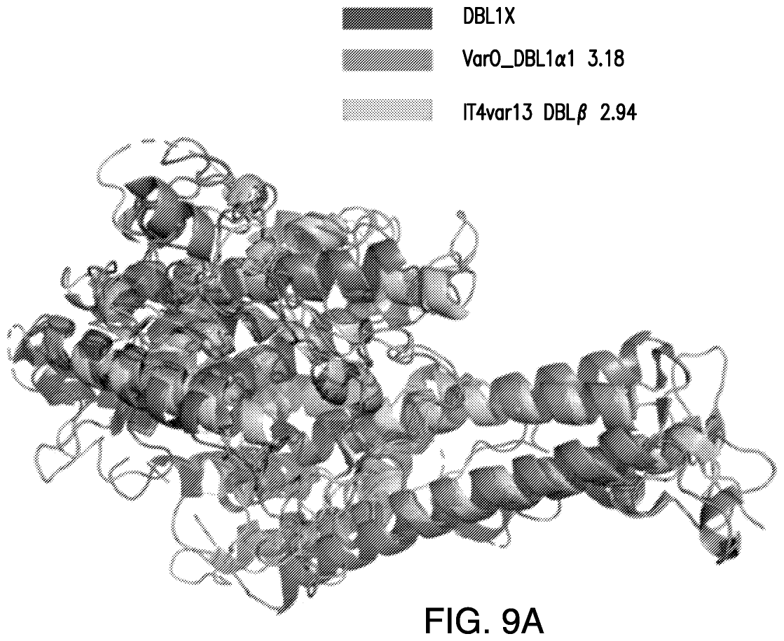

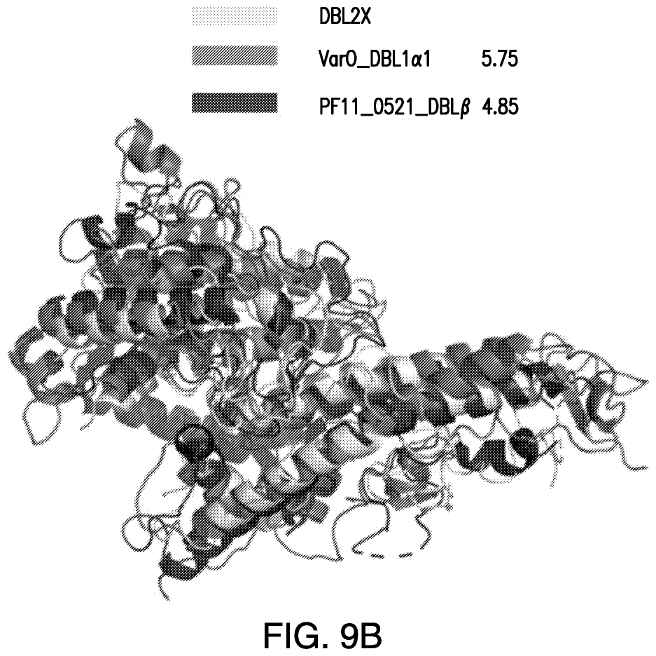

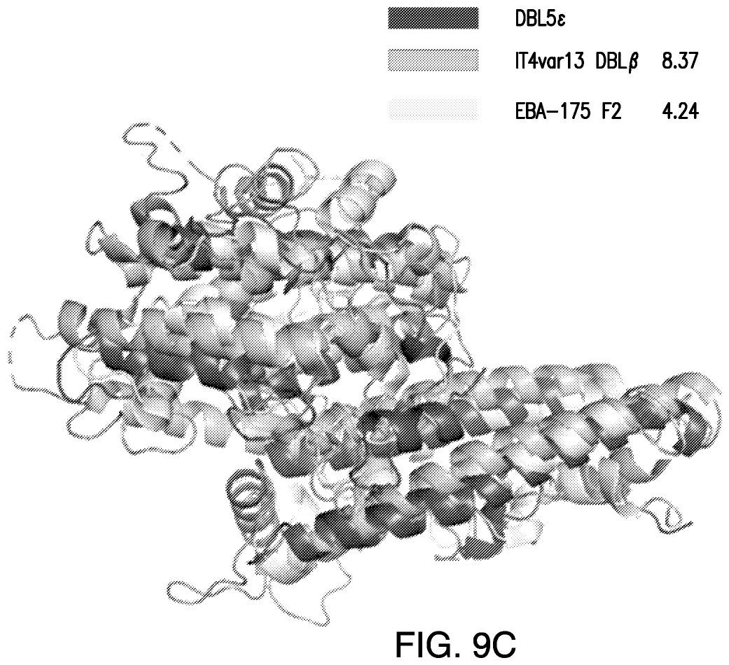

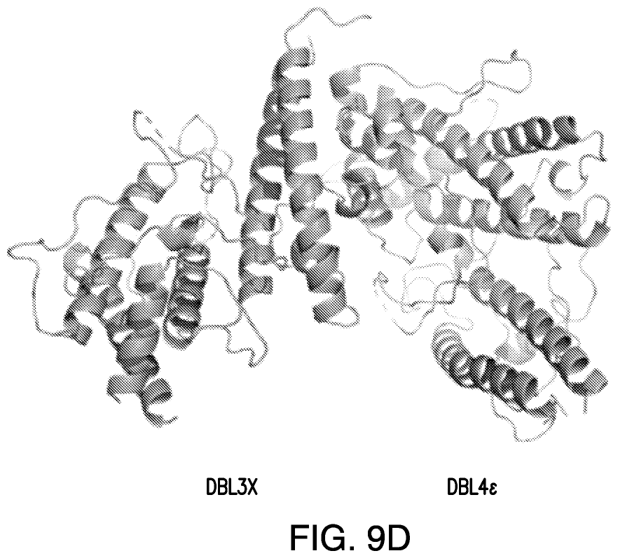

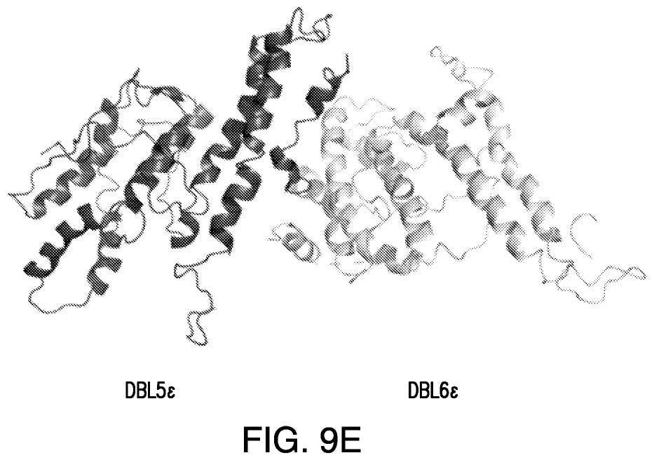

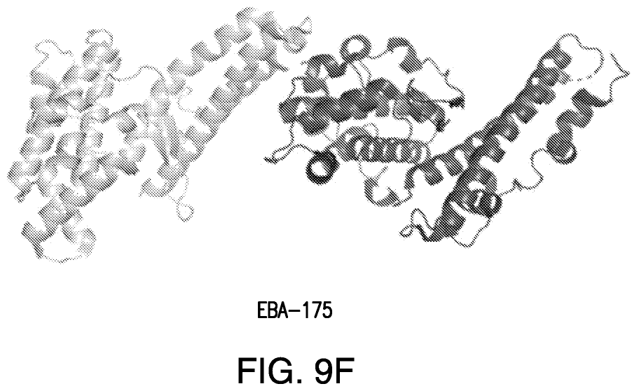

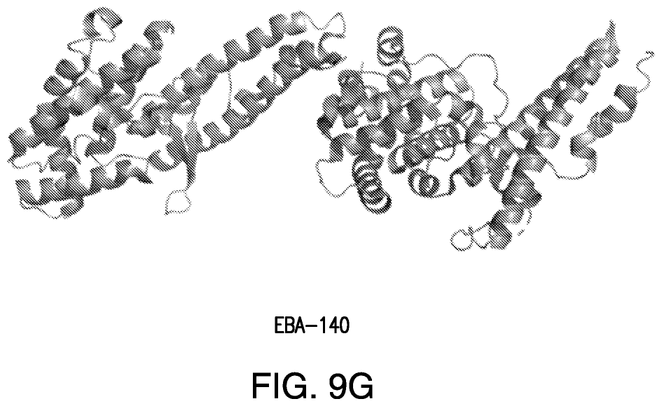

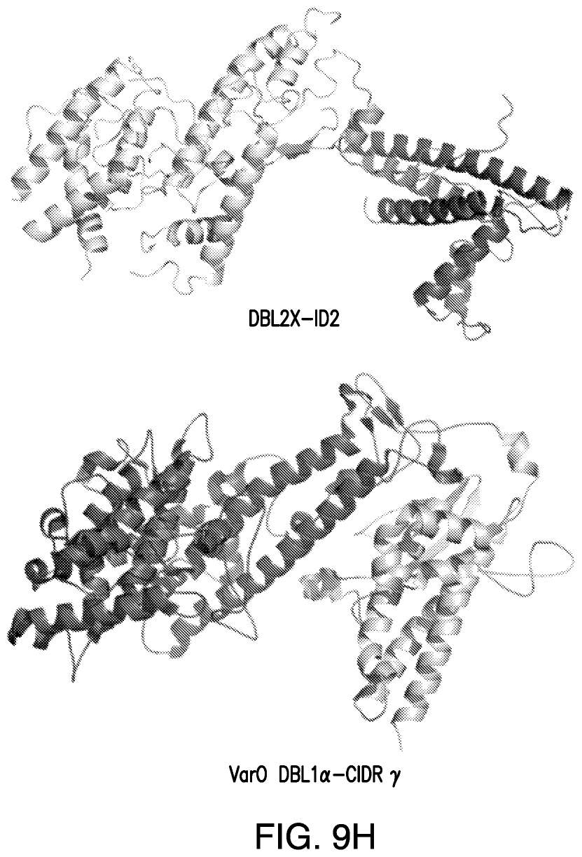

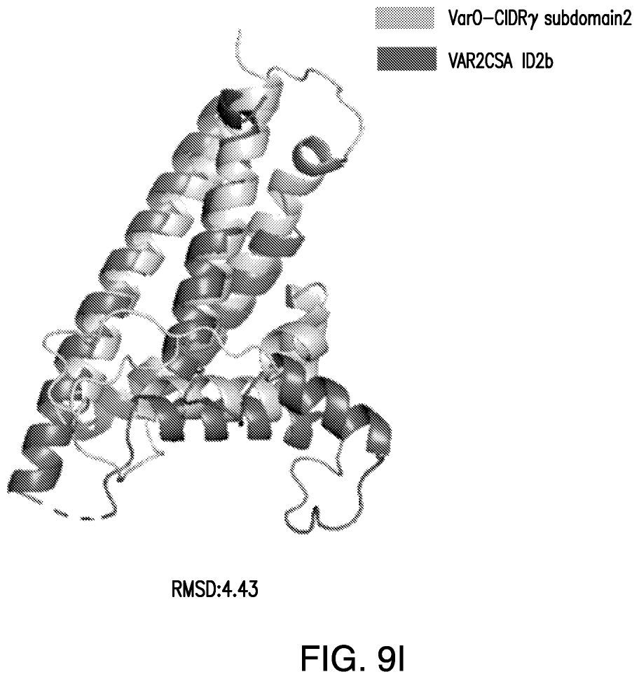

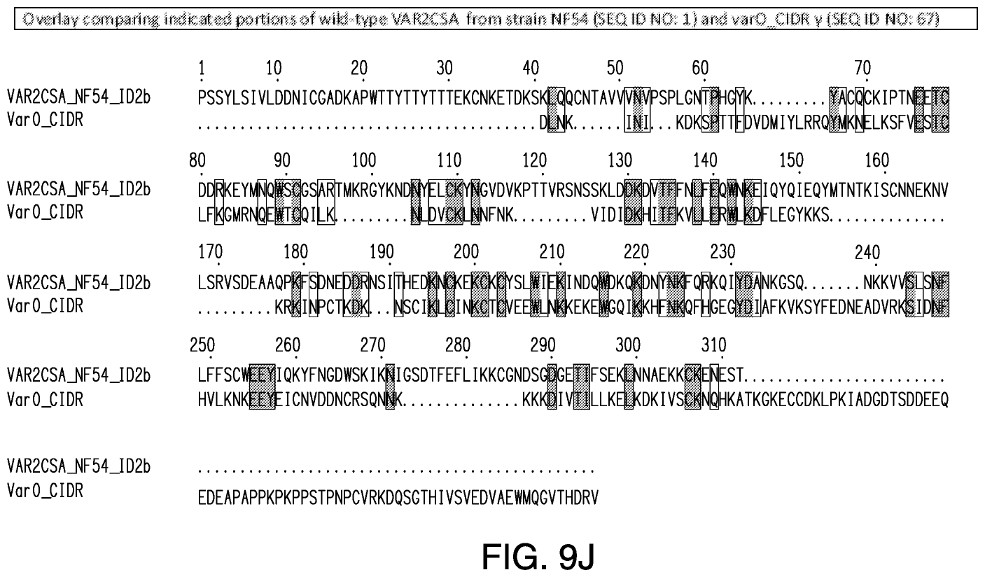

The VAR2CSA structure represents the first characterized structure of a full-length PfEMP1 protein, and provides the first structural models for DBL1X, DBL2X, ID2a, ID2b, ID3 and DBL5ε (FIG. 2a). Structural alignments were performed for these domains using the DALI search. DBL1X, DBL2X and DBL5ε adopt structures similar to other DBL domains from PfEMP1 and EBA-175 (FIG. 9a-c). A tandem packing of the dual DBL domains DBL3X/4ε and DBL5ε/6ε was also observed. These dual DBL domains exhibit a twisted pattern reminiscent of other tandem packed DBL pairs of EBA-140 and EBA-175, although the angle between DBL domains differs (FIG. 9d-g). EBA-140 and EBA-175 both belong to the EBL family that mediates the recognition of sialic acid on erythrocyte glycoproteins.

The arrangement of DBL2X-ID2 represents a conserved architecture within the PfEMP1 protein family. The structure of DBL2X-ID2 from VAR2CSA is similar to the DBL1α-CIDRγ domains of PfEMP1-VarO, although they adopt different DBL-ID/CIDR orientation (FIG. 9h). VarO binds the ABO blood group trisaccharide that mediates rosetting of infected red blood cells. The individual DBL domains (DBL2X and DBL1α) are structurally similar, and the VAR2CSA ID2b domain has a strong similarity to the VarO CIDRγ subdomain 2 despite low sequence similarity (FIG. 9b, I, and j). The DBL-ID/CIDR angle differs between VAR2CSA and PfEMP1-VarO, but this tandem arrangement suggests that the DBL-ID/CIDR pairing among other PfEMP1 family members may have a similar architecture. These structural delineations will better inform and define the diverse PfElVIP1 domain architectures.

### Multiple Domains Within the Core Domains Create Major and Minor CSA-Binding Channels

The atomic resolution reconstructions provided assignment of a CSA polymer comprising twelve monomers bound in a positively charged channel that is formed by NTS, DBL1X, DBL2X and DBL4ε. (FIG. 3a,b). This channel is referred to herein as the major CSA binding channel. Five sulfated N-acetylgalactosamine-4-sulfate (ASG) and six glucuronic acid (BDP) residues were assigned and built into the density. Furthermore, density for an additional residue is observed at the start of the chain that can accommodate a monosaccharide, but this density was not of sufficient quality to facilitate adequate modeling of this single residue (FIG. 3a).

The major binding channel can be separated into two non-continuous CSA-binding sites (FIG. 3a). The first binding site (major binding site 1) is located on the surface of DBL2X and binds CSA residues BDP-8 to ASG-11 (FIG. 3a, c, d). The sulfate group of ASG-11 forms hydrogen bonds with N557 while BDP-10 has interactions with R829, K561 and the main chain of A822 (FIG. 3c). ASG-9 forms multiple hydrogen bonds with K562, N576, K828 and Q832 (FIG. 3d). The interaction of CSA with major binding site 1 is further strengthened by the hydrogen bonds between BDP-8 and K828 (FIG. 3d).

The second binding site (major binding site 2) lies deep in the hole of the funnel-shaped channel and is surrounded by NTS, DBL1X, DBL2X and DBL4ε (FIG. 3a, b). Multiple hydrogen bonds are also formed in this region: ASG-5 with K835, E1880 and K1889; BDP-4 with K48; ASG-3 with K48 and R846; BDP-2 with R846 and the main chain of 11785 (FIG. 3e). Y45 further stabilizes the interaction by packing tightly with BDP-2 (FIG. 3e). BDP-6 and ASG-7 do not exhibit direct interactions with VAR2CSA and may serve to link the two binding sites together (FIG. 3f).

Weak density (the size of a single ASG monosaccharide) was also identified in a separate region of VAR2CSA, which is referred to herein as the minor binding channel that forms a potential second binding site (FIG. 10a). The minor binding channel is made up of the residues from the C-terminus of DBL2X and N-terminus of ID2a, two regions previously implicated in CSA binding. Similar to the major binding channel, the minor channel is rich in positively charged residues (FIG. 10b).

The CSA-binding residues in both channels are highly conserved among different VAR2CSA alleles (FIG. 3g and FIG. 10c). In addition, while individual segments of VAR2CSA demonstrate CSA binding, the full-length protein binds CSA with far greater affinity than any segment alone. Without wishing to be bound by a particular theory, the structure described above provides rationale for these observations. DBL1X, DBL2X, ID2a, ID2b, DBL3X, DBL4ε and ID3 all interact extensively to create an interwoven architecture (FIG. 1b, d). The CSA binding is likely dependent on an intact core structure implicating multiple domains in high affinity CSA binding.

### VAR2CSA Adopts Preformed CSA-Binding Channels

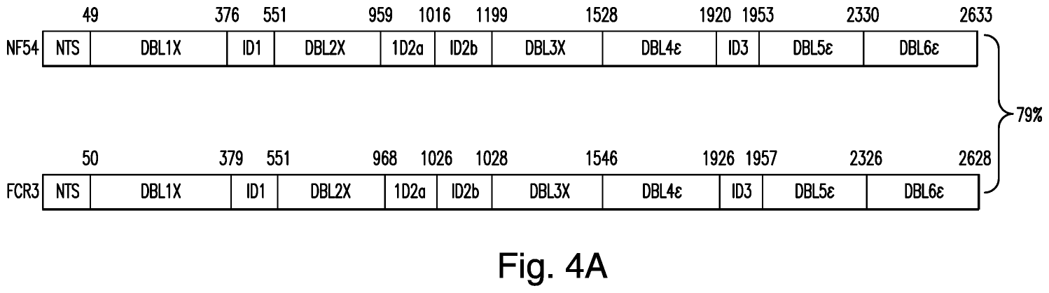

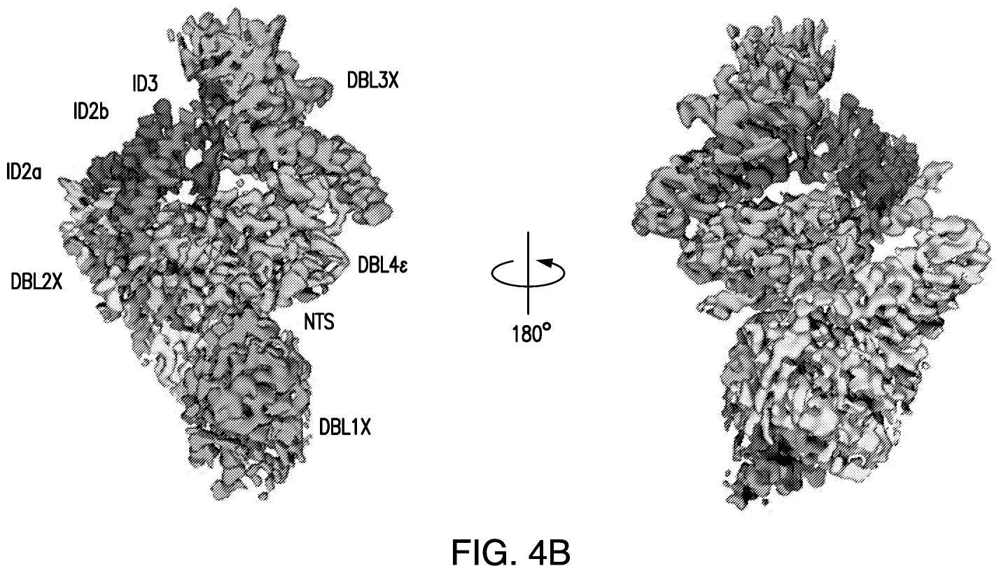

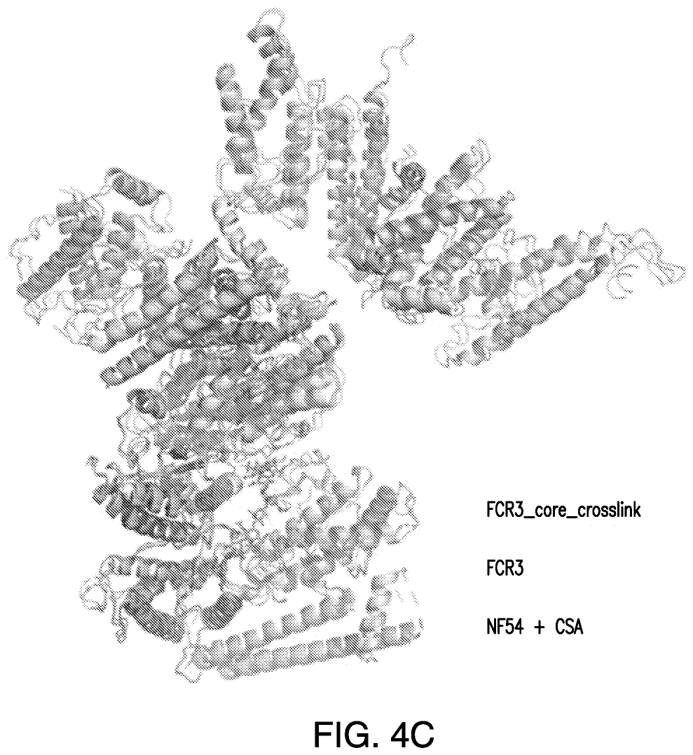

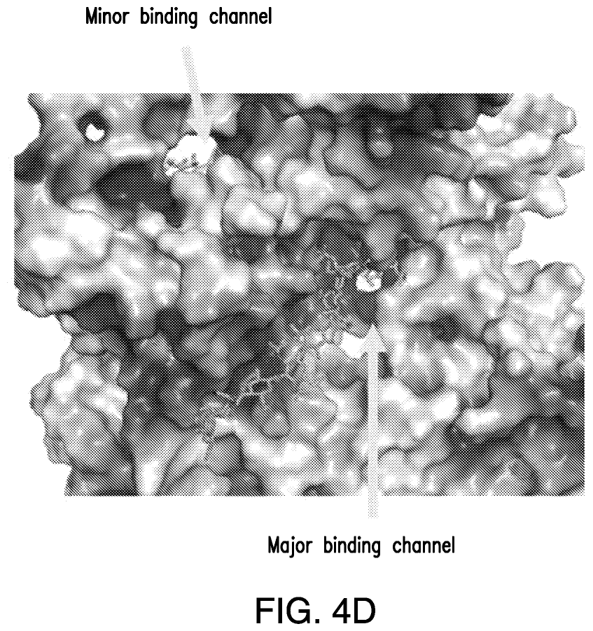

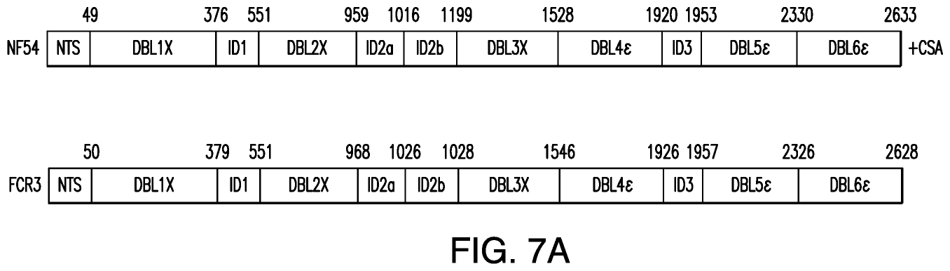

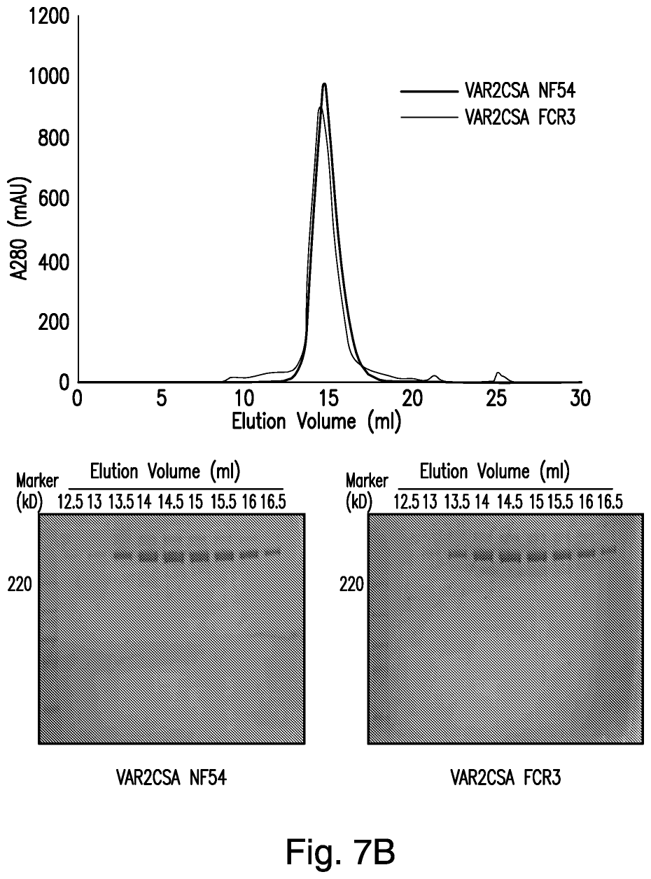

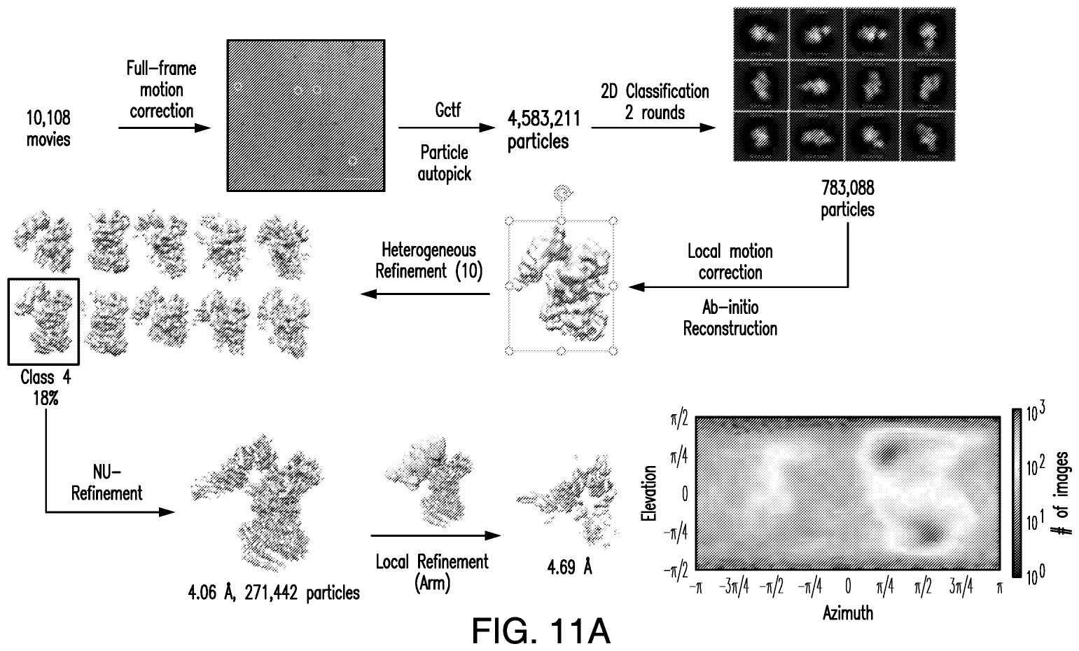

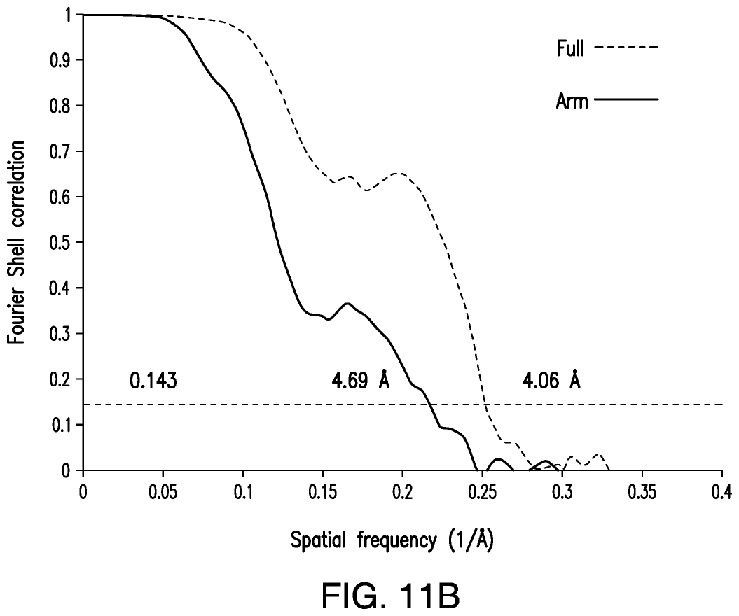

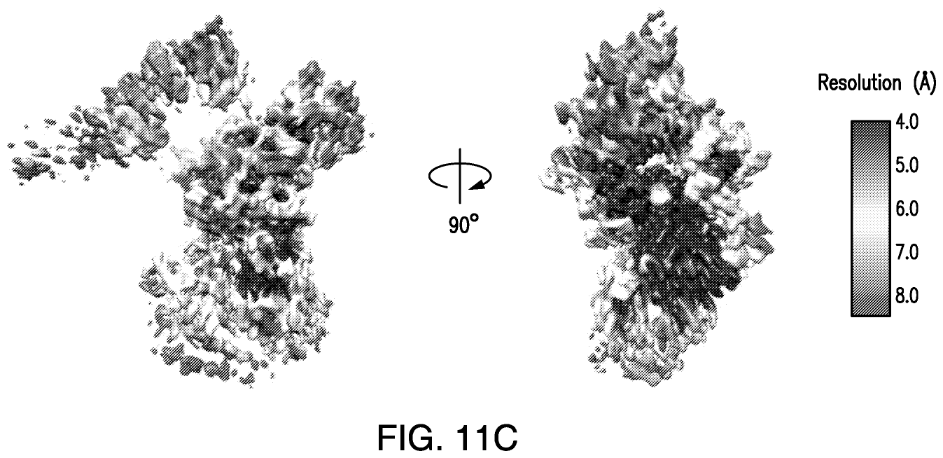

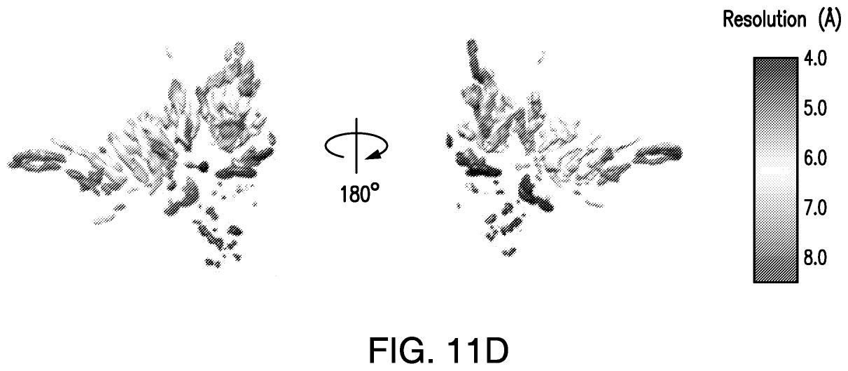

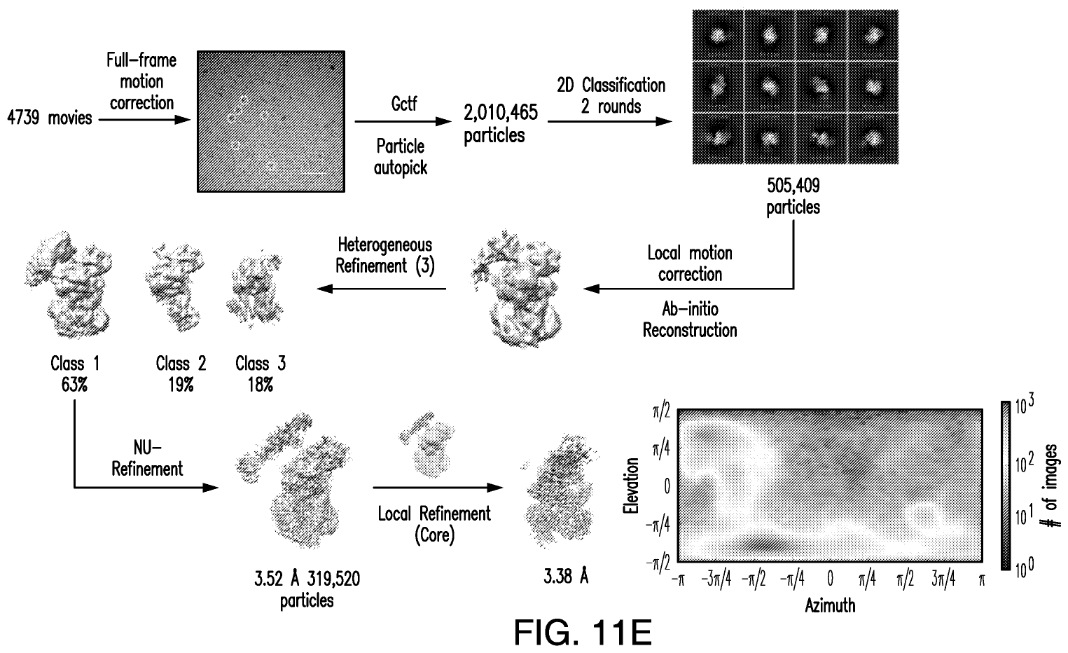

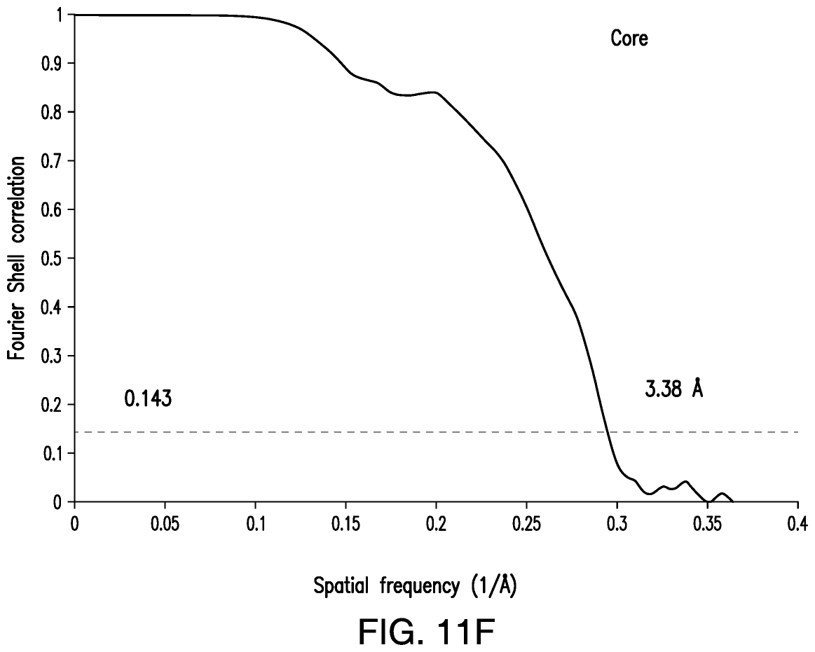

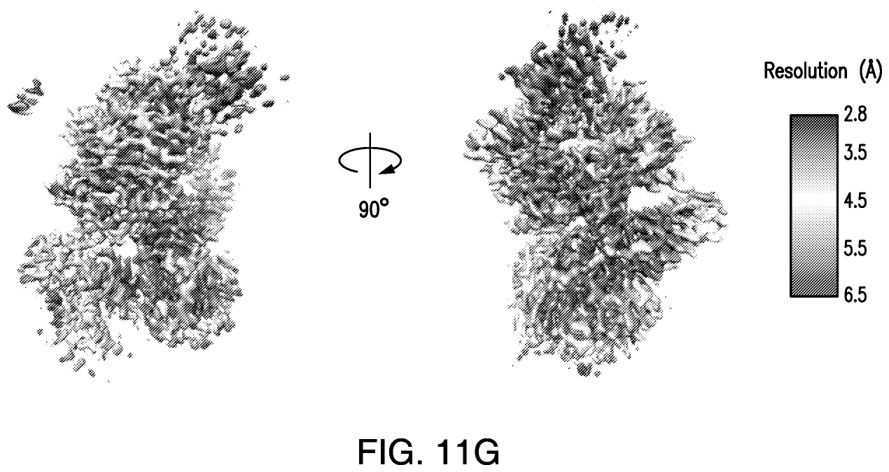

In addition to the CSA-VAR2CSA complex, the structure of CSA-free VAR2CSA from the parasite strain FCR3 was also solved (FIGS. 7 and 11 and Table 2). The sequence of these two VAR2CSA alleles share a 79% identity (FIG. 4a). The structure of VAR2CSA FCR3 may potentially inform development of a strain-transcending vaccine by revealing any conformational changes due to CSA binding, as well as commonalities and differences between strains.

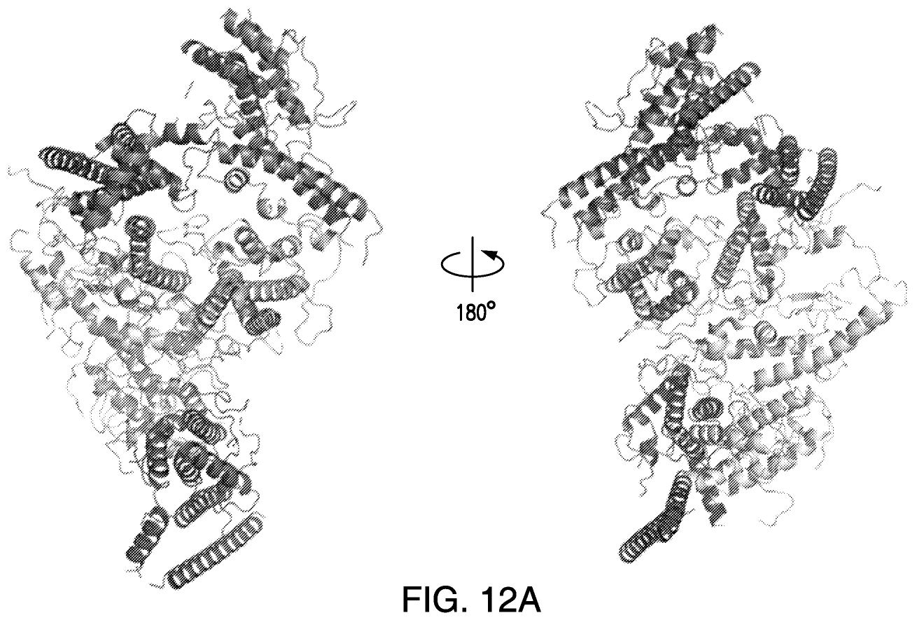

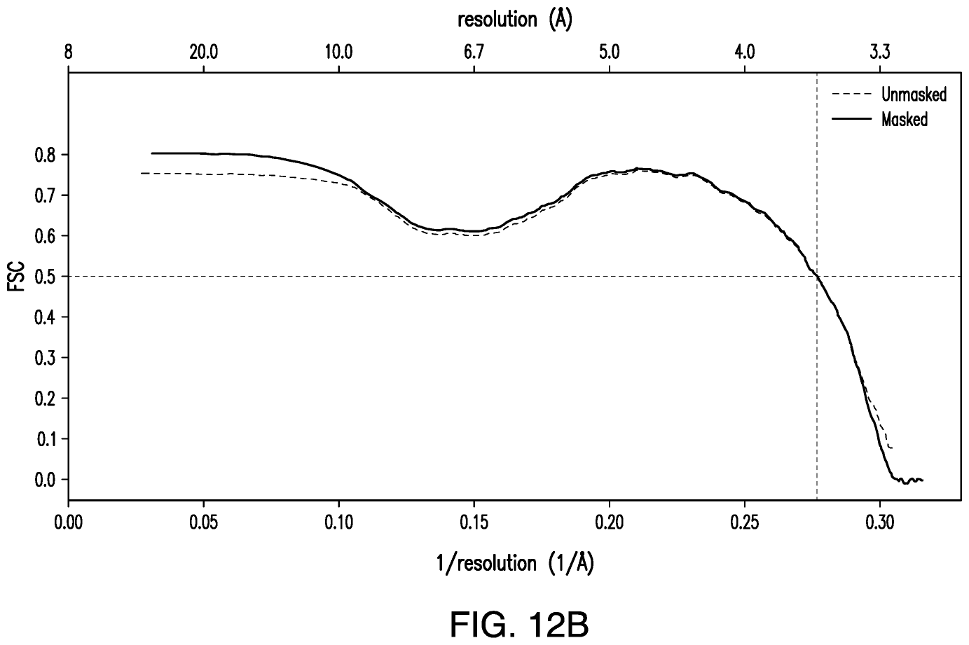

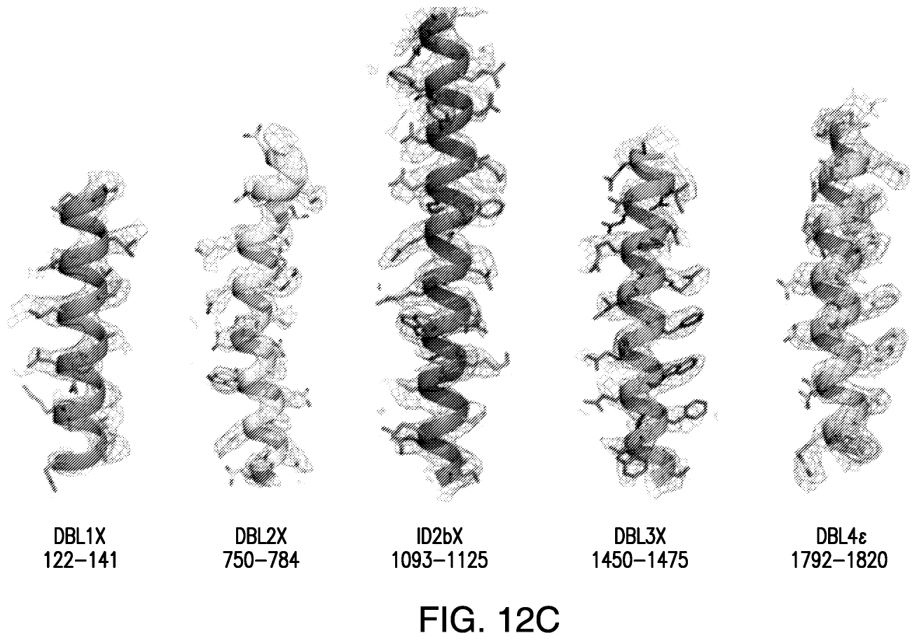

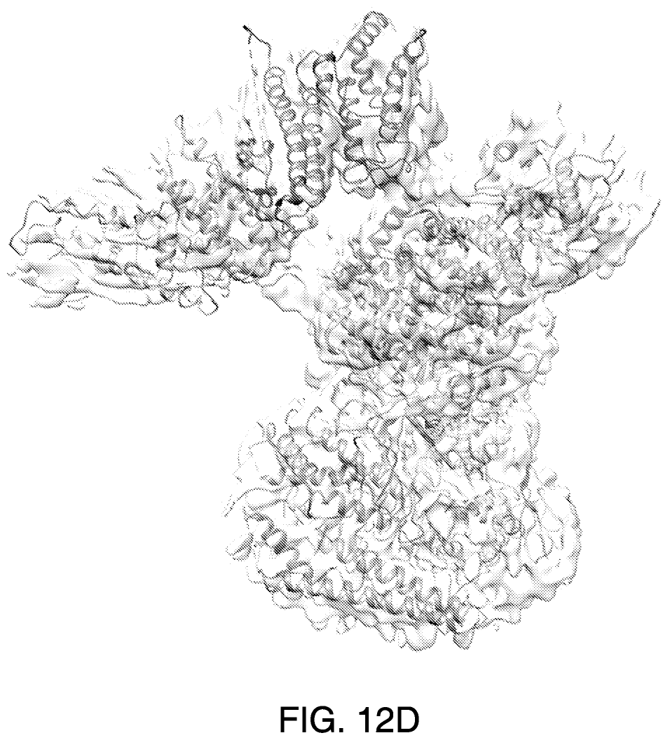

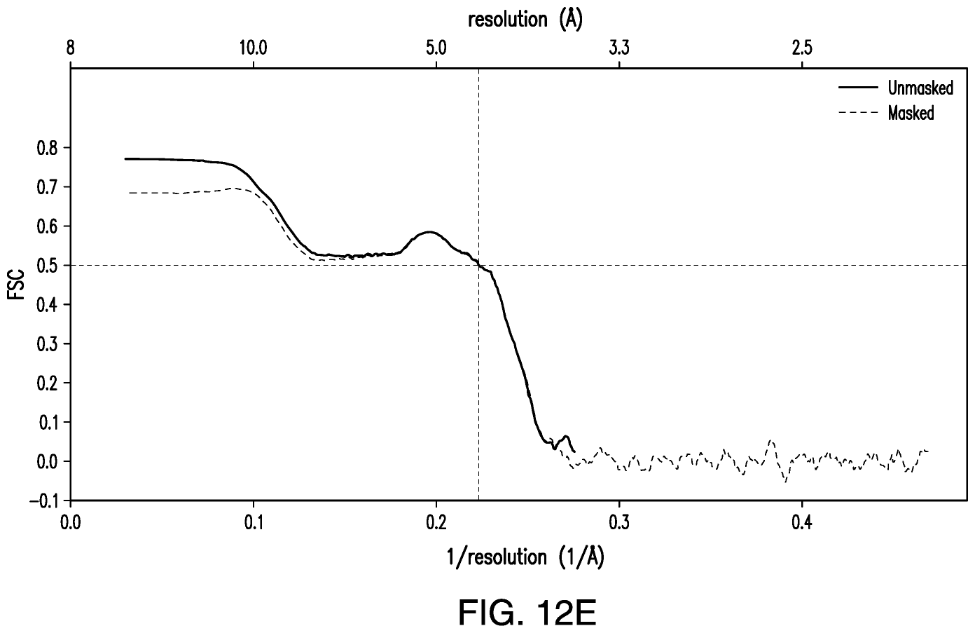

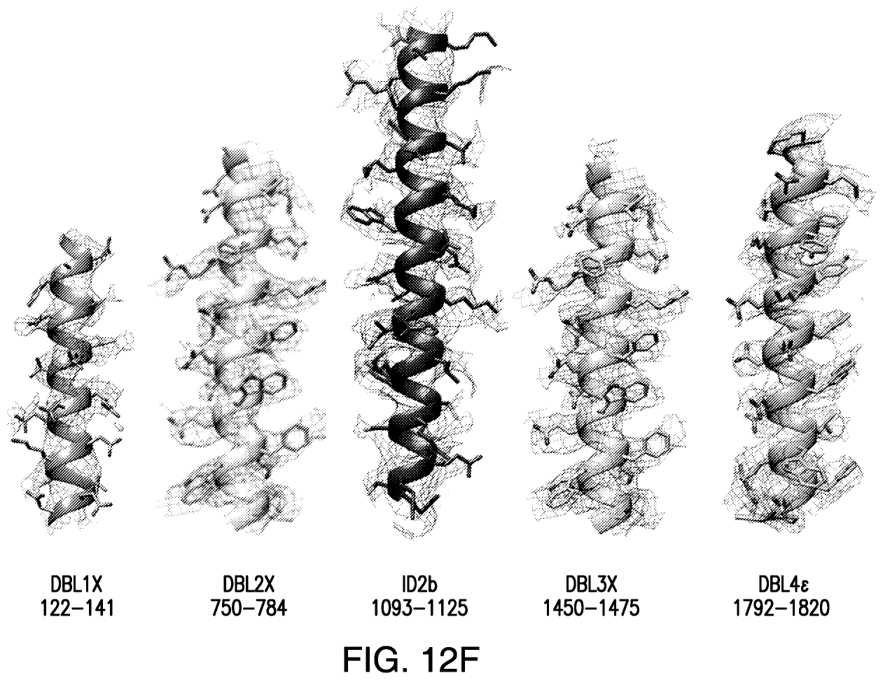

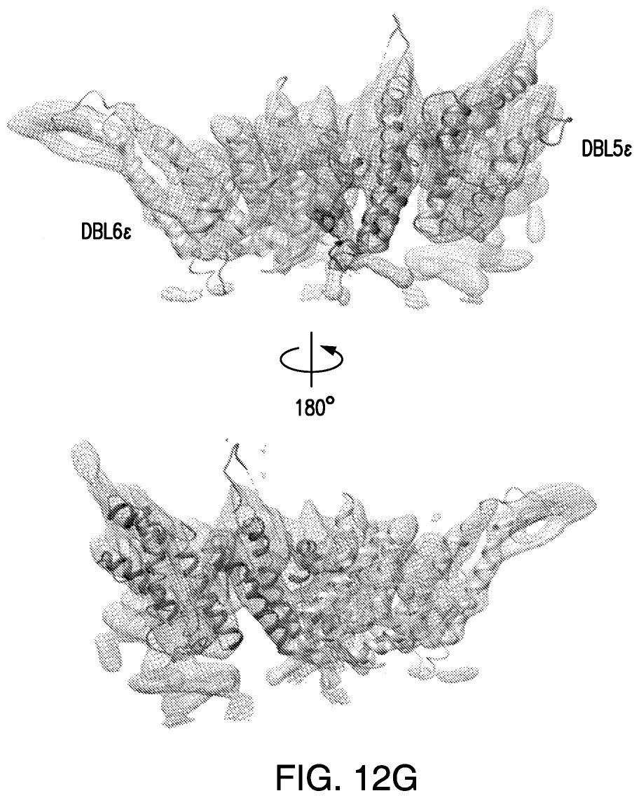

The Cryo-EM structure of the CSA-free full-length VAR2CSA FCR3 to a resolution of 4 Å after collecting 10,108 movies (FIG. 11a-c). The reconstructed map of apo VAR2CSA FCR3 exhibits a similar shape to the CSA bound VAR2CSA, and also resembles the number 7 with a stable core and flexible arm. Local refinement of the arm resulted in a 4.7 Å map (FIG. 11d). In order to further improve the resolution and the accuracy, the full-length ectodomain was cross-linked under mild conditions and collected a second dataset of 4,739 micrograph movies. This dataset resulted in a reconstruction of the stable core comprising DBL1X to ID3 to 3.4 Å resolution, enabling accurate model building for this segment that comprises the majority of VAR2CSA (FIG. 4b; 11e-g, 12, Table 2). The 4.7 Å reconstruction from masked local refinement of DBL5ε and 6ε allowed docking and refinement of the C-alpha positions of DBL5ε as well as the available crystal structure of DBL6ε (PDB:2Y8D) into this map (FIG. 12g). The final model for VAR2CSA FCR3 spans residues 23 to 2602 of VAR2CSA with a few flexible loops and ID1 omitted, as these segments were not ordered in the reconstruction (FIG. 12d). Comparison of the DBL1X-ID3 map generated from the cross-linked and non-cross-linked sample reveals no noticeable conformational changes in the core, indicating the crosslink did not affect conformation (FIG. 4c).

No major conformation changes were observed between the structures of CSA-bound and CSA-free VAR2CSA (FIG. 4c). The structural similarity between VAR2CSA FCR3 and NF54 also suggests different VAR2CSA variants are likely to have similar overall architecture (FIG. 4c). CSA could be well docked in the corresponding major and minor binding channels on VAR2CSA FCR3, which is similarly positively charged (FIG. 4c, d). This suggests that the CSA-binding mode identified herein is conserved between strains, and that VAR2CSA does not require major conformational changes to enable CSA binding. However, some flexibility is observed in the region DBL1X-DBL2X, suggesting limited flexing of the molecule may facilitate CSA binding.

### Analysis of VAR2CSA Variability and Placental Malaria Candidate Vaccine Designs

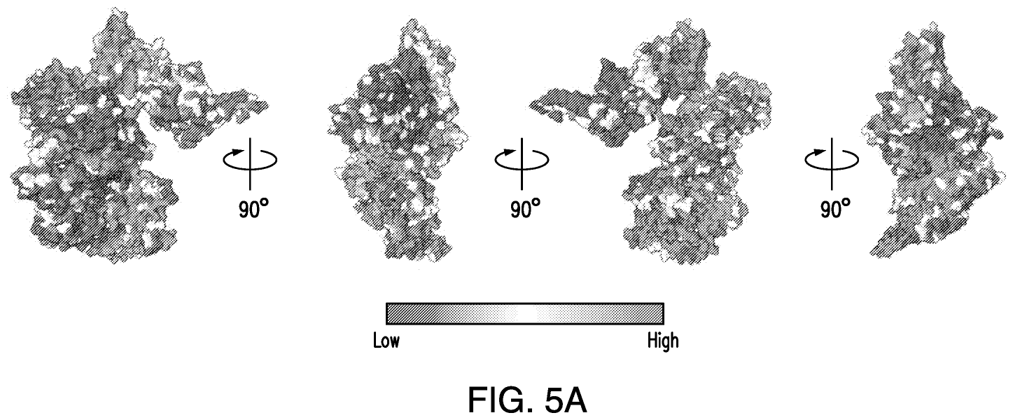

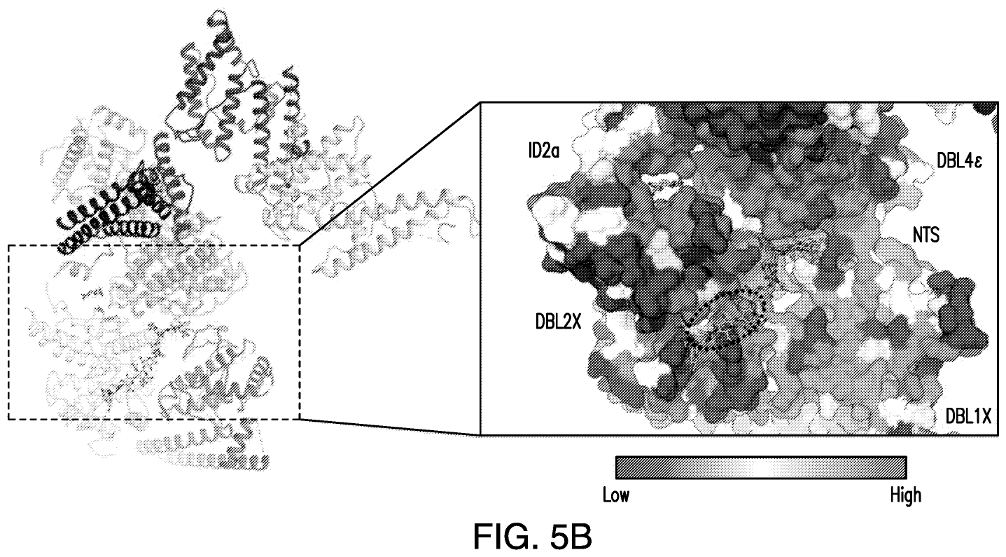

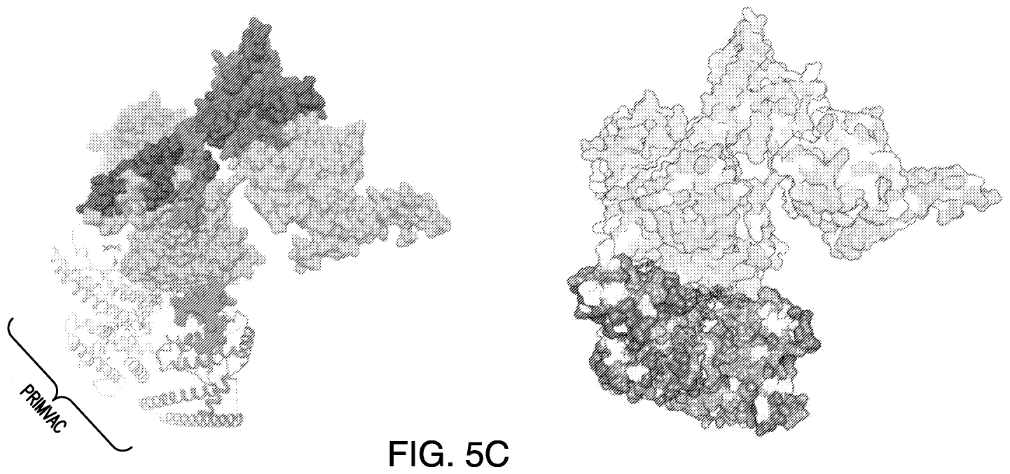

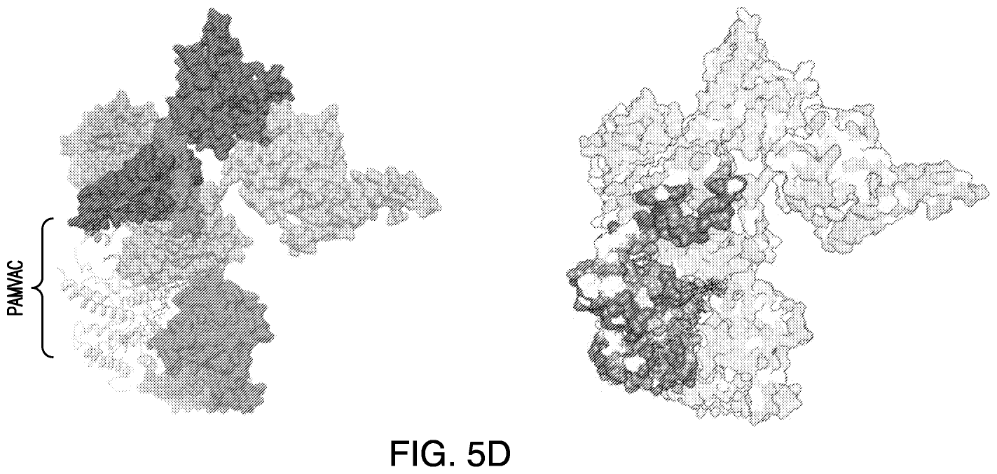

High sequence polymorphism among diverse VAR2CSA variants is one of the major barriers to strain-transcending vaccine development. The conservation of fourteen VAR2CSA sequences was analyzed and mapped onto the structure (FIG. 5a). Residues in the CSA-binding sites within the major and minor binding channels are conserved, but the flanking regions are not (FIG. 5b). Without wishing to be bound by a particular theory, the high conservation of the residues within both channels that directly bind CSA indicate these residues are under selective pressure to be maintained across strains. These results suggest that all strains retain these residues to ensure CSA binding. The variability observed in the flanking regions that are distant from the CSA-binding residues and do not directly contact CSA suggests that variability at these positions should not impact CSA binding, but may play a role in immune evasion.

The CSA-binding site 2 is buried deeply in the major binding channel and may not be accessible to antibodies (FIG. 5b). Although binding site 1 is exposed on the VAR2CSA surface, the DBL2X surface surrounding the conserved CSA binding channel residues is highly heterogeneous among diverse VAR2CSA strains (FIG. 5b). Moreover, there is also extensive polymorphism surrounding the conserved residues within the minor CSA binding channel (FIG. 5b). Other than the key CSA-binding residues, a large number of the surface residues are polymorphic among different VAR2CSA strains (FIG. 5a).

The interwoven domain architecture identified in the structure is consistent with the finding that multiple domains play a role in binding CSA, as multiple domains create the binding channels. The structure of full-length VAR2CSA described herein reveals larger CSA-binding sites with conserved targets for strain-transcending antibodies. This information will guide improvements on existing candidate vaccines and facilitate structure-based design of a strain-transcending placental malaria vaccine.

### Epitope Mapping on VAR2CSA

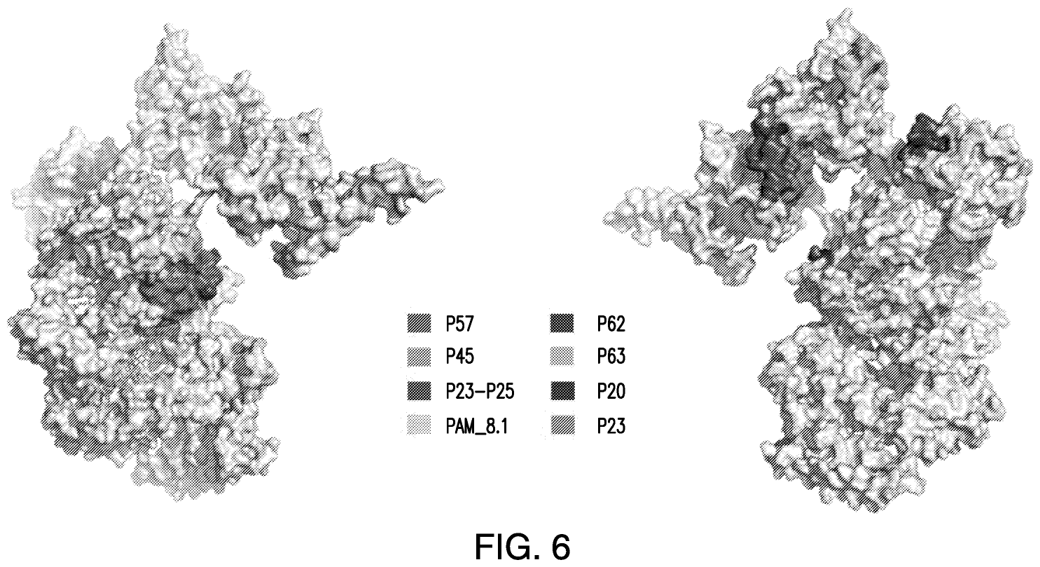

The structure of full-length VAR2CSA provided a template to investigate previously discovered antibody epitopes. Known epitopes were mapped on the structure (FIG. 6). Four multigravidae sera with neutralizing activity showed enhanced binding to distinct linear peptides using overlapping peptide scanning of DBL4ε. All the sera showed antibody binding to peptides P23-P25 and one sample also showed reactivity to peptides P45 and P57. Interestingly, mapping of these peptides on the 3D structure revealed all these peptides cluster together and are located at the entrance of the deeply buried binding site 2 of the major CSA binding channel (FIG. 6). Separately, naturally acquired antibodies to ID1-DBL2-ID2a and DBL4 recombinant constructs were found to have neutralizing activity against both homologous and heterologous isolates, and these results are consistent with the structural analysis identifying these domains as important for CSA binding. Other known epitopes of antibodies from multigravid women were mapped (FIG. 6). The epitopes of PAM8.1, which is an antibody derived from multigravid woman, was mapped to a strain-specific loop region on DBL3X. However, this loop is not visible in the structure (FIG. 6). Peptide P62 found within DBL3X and peptide P63 within DBL5ε are two peptides that react strongly with Tanzanian female plasma. Lastly, peptides P20 and P23 are two cryptic epitopes on DBL5ε which are shown to cross-react with the antibodies derived from Plasmodium vivax DBP (PvDBP). However, the neutralizing mechanisms of the antibodies that react to these epitopes requires further study.

### Discussion

The Cryo-EM structure of VAR2CSA in CSA-bound and CSA-free states determined here support a model of binding depicted in FIG. 6a. A major CSA-binding channel was identified that has two non-continuous CSA-binding sites, and a potential minor CSA-binding channel on VAR2CSA was identified, both of which are pre-formed by multiple domains. Although the CSA binding channel residues are highly conserved among various VAR2CSA alleles, a few CSA binding channel residues at the extremities of the binding sites exhibit polymorphism (FIG. 3g). In addition, the conserved residues are flanked by highly polymorphic residues (FIG. 5b). These variabilities may contribute to diverse binding affinity and PM severity of various VAR2CSA isolates. The surface-exposed binding site 1 of the major binding channel is formed solely by DBL2X (FIG. 3a). The buried binding site 2 of the major binding channel and the minor binding channel are formed by the NTS, DBL1X, DBL2X, ID2a and DBL4ε domains (FIG. 3a). The finding that DBL2X appears in all CSA-binding sites suggests its central role in CSA binding.

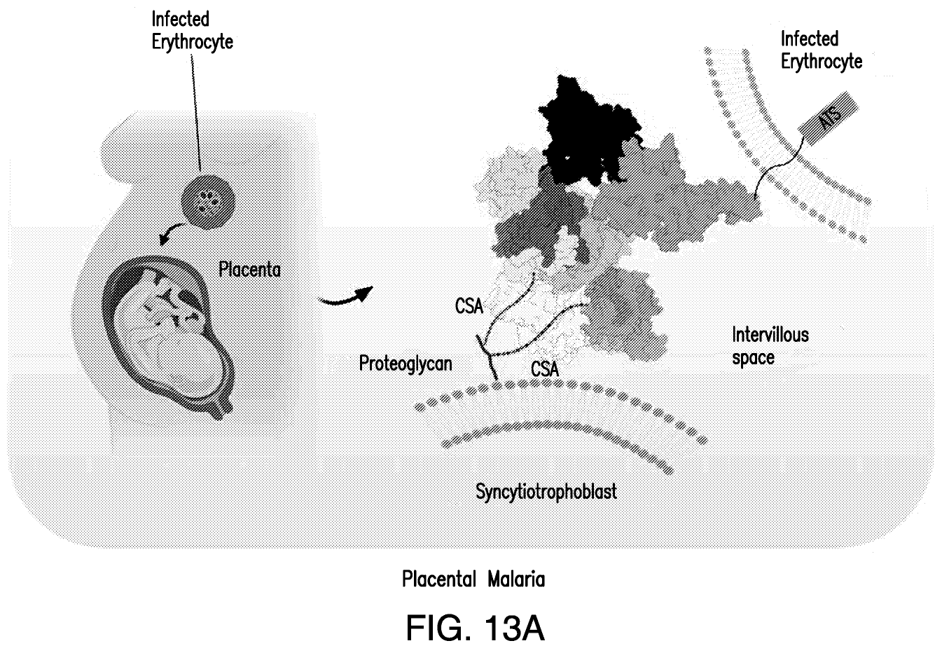

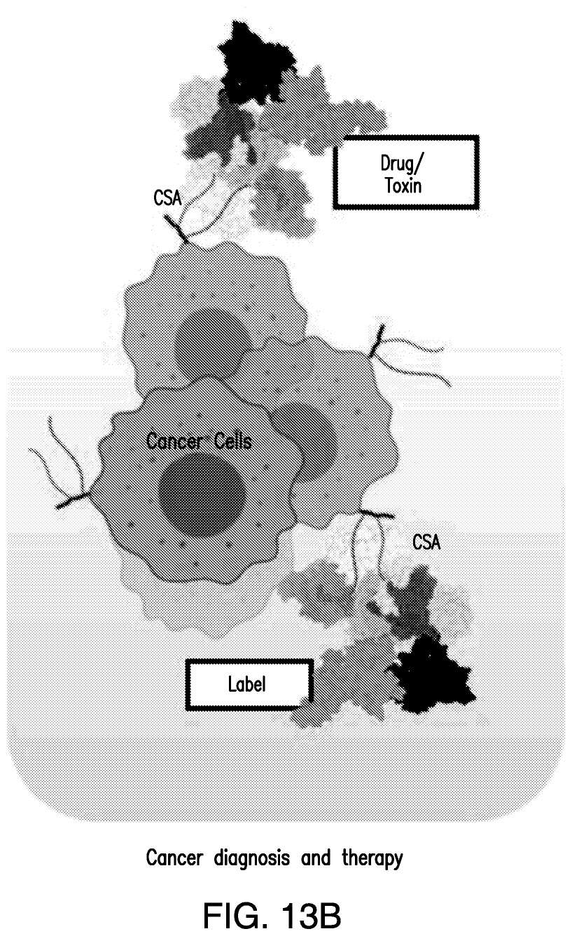

However, the multidomain binding model identifies all the CSA-binding regions and explains why the full-length VAR2CSA has much stronger CSA-binding affinity than any individual or short continuous domains (FIG. 13a). This study also identified DBL4ε as a key component of the CSA-binding channel. The binding residues of DBL4ε are buried in the hole of the channel and they work together with segments from the NTS, DBL1X and DBL2X to form the binding site 2 of the major binding channel.

The similar overall architecture of VAR2CSA from parasite strains NF54 and FCR3 implies that the VAR2CSA adopts a conserved shape. Some VAR2CSA proteins have been shown to have an additional DBL domain termed DBL7ε. This DBL domain would be connected to the C-terminus of DBL6ε that is fully solvent exposed and away from both the arm and core of all structures reported here. DBL7ε can readily be accommodated as an extension of the arm and is unlikely to alter the remaining architecture of VAR2CSA.

In the above study, CSA from bovine trachea was used, which consists of a mixture of CSA with different sulfation patterns and of different lengths. Although five of the CSA disaccharides are fully sulfated in the structure, the sulfation status of the first CSA monosaccharide was not determined. Since the CSA completely traverses through the binding channel of VAR2CSA, it is also plausible that VAR2CSA may slide along a CSA chain to search for a highly sulfated cluster prior to strong binding. Furthermore, the fact that CSA is tethered to the proteoglycan in the placenta might facilitate the binding of multiple CSA glycans to the different CSA binding channels on one VAR2CSA molecule or distinct VAR2CSA molecules that are located on a same knob.

The high variability of VAR2CSA from distinct P. falciparum strains poses a challenge to the development of strain-transcending vaccines for placental malaria. Mapping the VAR2CSA sequence variability onto the 3D structure of VAR2CSA shows the CSA-binding site 1 on DBL2X is highly conserved but is surrounded by highly polymorphic residues (FIG. 5b). This explains the low heterogenous neutralizing activity observed for the placental malaria candidate vaccines. The highly polymorphic segments likely impact antibodies that bind at or close to the CSA-binding sites, preventing the development of antibodies capable of binding to the VAR2CSA variants. The structure of VAR2CSA bound to CSA presented here serves as a template to design and develop vaccines against placental malaria that will overcome strain-specific responses by focusing the immune response to conserved regions.

Multiple pieces of evidence suggest the immunogens that encompass the region NTS-DBL2X can bind to antibodies from multigravid women living in pandemic regions, and can induce protective antibodies in clinical trials. The previously identified linear peptide epitopes on DBL4ε reside right next the major CSA binding channel. Accordingly, it is theorized that these linear peptides may be part of larger conformational epitopes that target the major CSA binding channel.

Interestingly, the form of CSA bound by VAR2CSA is exclusively expressed in the placenta in healthy individuals, but is expressed and presented in cells from diverse cancers of epithelial and mesenchymal origin. This expression allows for the specific targeting of cancer cells by delivering therapeutics that utilize VAR2CSA as a carrier, and for VAR2CSA-based cancer diagnostics. Clear structural definition of the functional segments from VAR2CSA required to bind CSA will lead to improvements for placental malaria vaccine development as well as cancer therapeutics and diagnostics (FIG. 13b). Improving the affinity of VAR2CSA fragments for cancer therapy by structure-guided design may allow for improved treatments that require lower doses for efficacy.

In summary, this example describes a study of VAR2CSA that defines the CSA-binding elements that comprise conserved segments of VAR2CSA to target for strain-transcending protective immunity. This information supports precise design of vaccines to provide much needed medical countermeasures against placental malaria and will inform the development of potent targeted cancer therapeutics and diagnostics.

### EXAMPLE 2

This example describes design and characterization of immunogen polypeptides comprising the CSA binding regions of VAR2CSA.

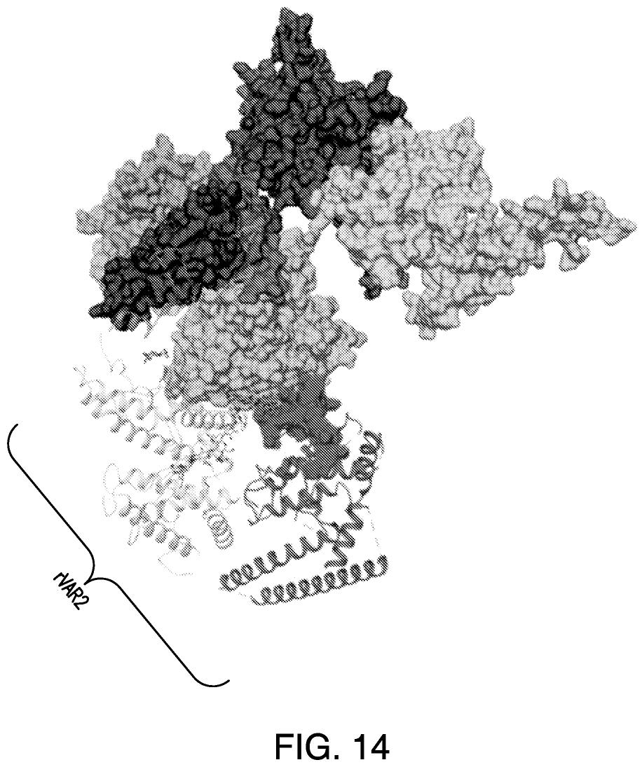

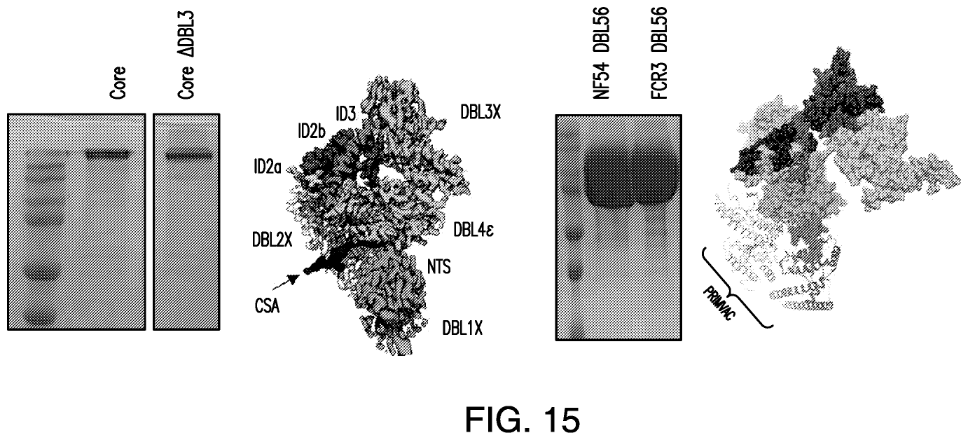

Sequences (SEQ ID NOS 3-54) were designed based on the structure of VAR2CSA elucidated by the inventors. These sequences are not found in nature. Photographs depicting the results of electrophoresis for the NF54 Core ΔDBL3 (“major and minor channels”, SEQ ID NO: 3), NF54 Core (SEQ ID NO: 5), NF54 DBL56 (“arm”, SEQ ID NO: 7), and FCR3 DBL56 (“arm”, SEQ ID NO: 8), are presented as FIG. 14.

The disclosed sequences may be used to produce recombinant immunogen polypeptides. Testing is contemplated to confirm that these peptides have improved affinity for CSA and may be used specifically for targeting CSA. The affinity will be further improved by development using multimers or nanoparticles to increase the avidity of binding. Disclosed sequences will be fused to polypeptide sequences that form multimers with 2 to 60 monomers per multimer. Improved CSA affinity will allow the immunogen polypeptides to specifically target CSA expressing cancer cell types.

Additionally, rodent studies is contemplated to confirm that the immunogen polypeptides induce an immune response, and allow test subjects to raise inhibitory antibodies.

### EXAMPLE 3

This example provides an overview of several novel immunogen polypeptides and investigations thereof according to the present disclosure.

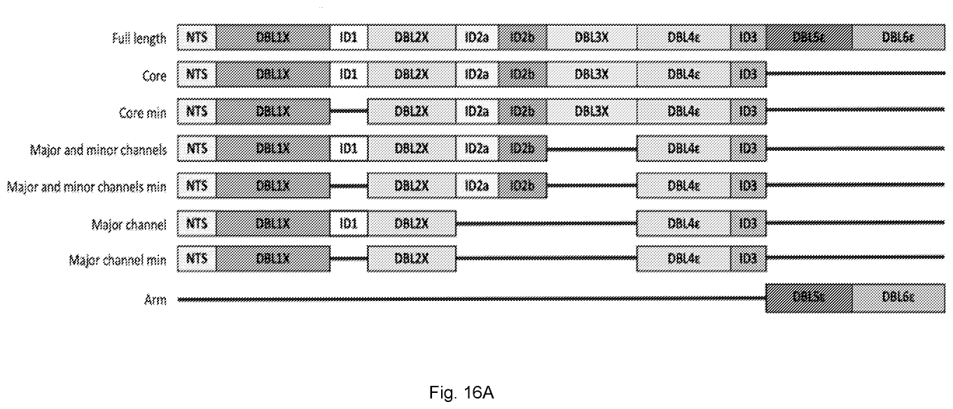

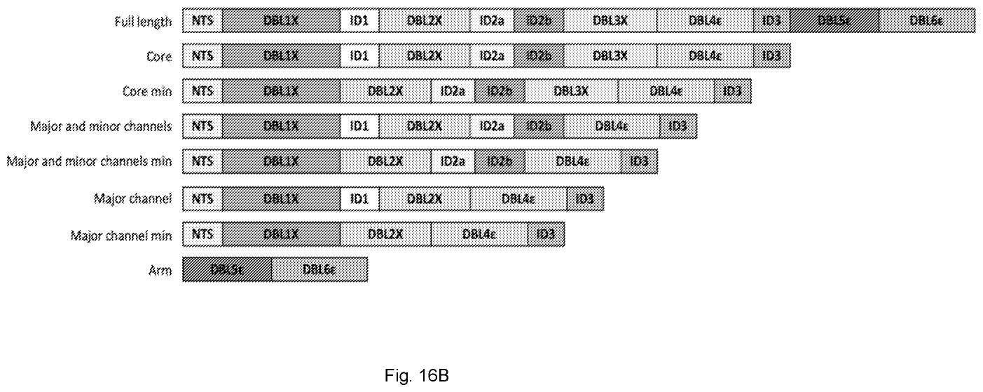

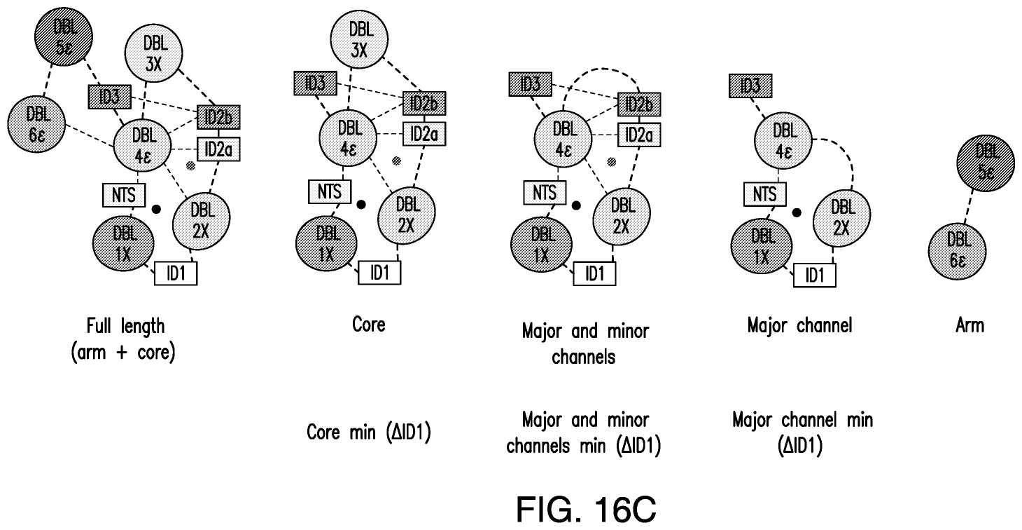

The inventors have designed and produced novel immunogen polypeptides from the P. falciparum strains NF54 and FCR3 (See FIGS. 16A-C). These designs have been evaluated in preclinical animal studies for their ability to raise antibodies that prevent adherence of P. falciparum-infected red blood cells to CSA in standardized assays.

Preclinical studies in rats in combination with in vitro assays for CSA-based adherence of infected red cells are the standard in the field to evaluate novel placental malaria candidates. Animal studies were conducted in rats on immunogen polypeptides derived from the NF54 strain. Serology studies indicated that all of the immunogen polypeptides raise antibody titers greater than adjuvant control, and equal or greater antibody titers than the full-length VAR2CSA ectodomain control after three vaccinations.

Three of these disclosed immunogen polypeptides in particular show potential for further development: the “core”, the “major and minor channel”, and the “major channel”. As will be further described in subsequent Examples, these immunogens elicit strong strain-transcending inhibitory activity upon vaccination of rats. Studies were performed using an adjuvant suitable for translation to clinical studies and form a strong foundation for the further development of these candidates as vaccines.

### EXAMPLE 4

This Example confirms that the disclosed immunogen polypeptides are stable can be produced at yields equal to or greater than the native full length VAR2CSA ectodomain.

Multiple design were developed based on the VAR2CSA cryoEM structures and mechanisms of CSA binding described herein. Designs were developed based on VAR2CSA from both the NF54 and FCR3 strains.

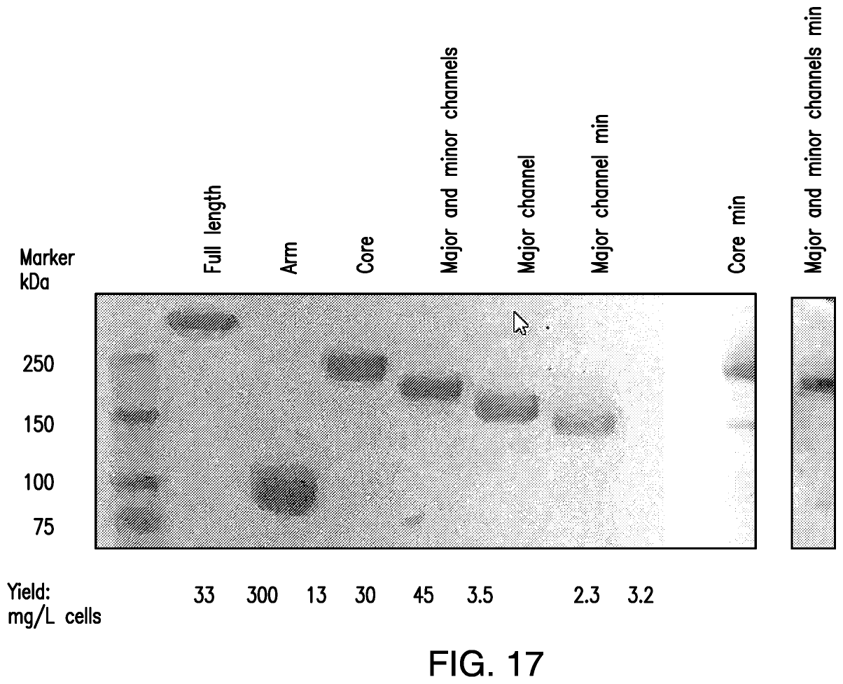

SDS page analysis of the purified immunogens for the NF54 strains proves high purity of the purified recombinant immunogens (FIG. 17). Notably, the yields of the three lead immunogens (Core, Major and minor channels, and Major channel) are equal to or better than the Full length construct.

### EXAMPLE 5

This Example demonstrates the evaluation of disclosed immunogen polypeptides' ability to bind directly to CSA.

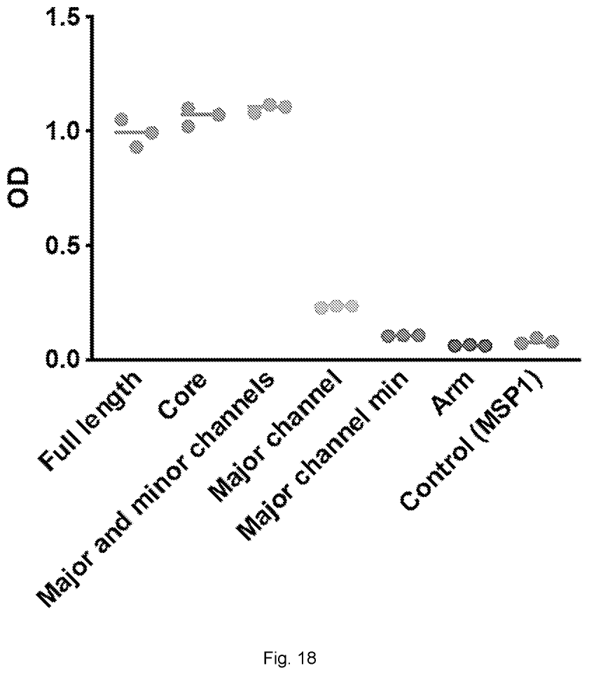

Structural and mechanistic studies suggest that the CSA binding activity is located in two channels within the Core domain (NTS to DBL4) of VAR2CSA, and the Arm segment (DBL5 and DBL6) is dispensable for binding. The ability of the protein designs to bind CSA was evaluated, and, consistent with the predicted model of binding, the core and the major and minor channels designs that contain both CSA-binding channels bound to CSA (FIG. 18). The Major channel design lacks one of the two CSA-binding channels (see FIGS. 16A and 16B) as expected binds CSA although binding is diminished compared to the Core and FL proteins (FIG. 18). The Arm did not bind CSA, again consistent with our model of binding where the domains in the Arm are not part of the CSA-binding channels. The Major channel min also lost binding suggesting the potential for an altered structure or lack of elements required for full binding.

### EXAMPLE 6

This Example demonstrates that the immunogenic polypeptides produce antibody titers greater than adjuvant control.

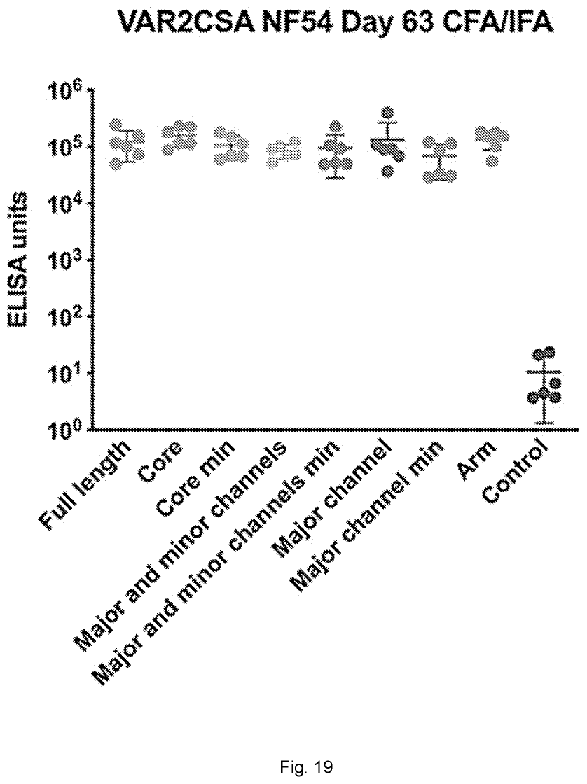

Rats are the model organism to evaluate placental malaria vaccine candidates. Rats were immunized with three doses of the disclosed immunogens using two different adjuvants, CFA/IFA and AddaS03. Vaccines were administered on days 0, 21 and 42, and samples were colleted on days 14, 35 and 63. All immunogens were first evaluated with CFA/IFA and those that showed inhibitory activity further tested using AddaS03 (see Example 8 below). Serology studies indicated that all immunogens raise antibody titers greater than adjuvant control, and equal to or greater than the full-length immunogen control after three vaccinations with CFA/IFA (FIG. 19).

### EXAMPLE 7

This Example demonstrates that disclosed immunogen polypeptides elicit antibodies that inhibit the homologous parasite strain.

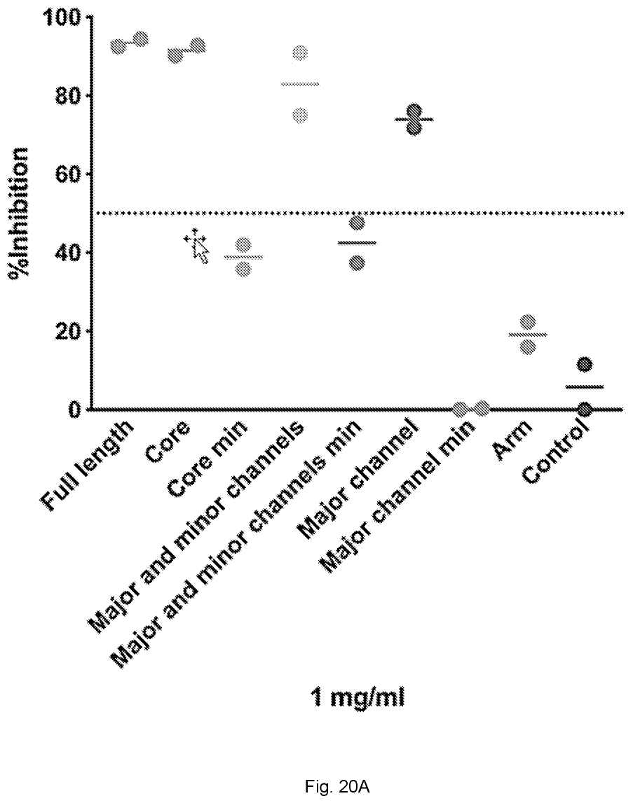

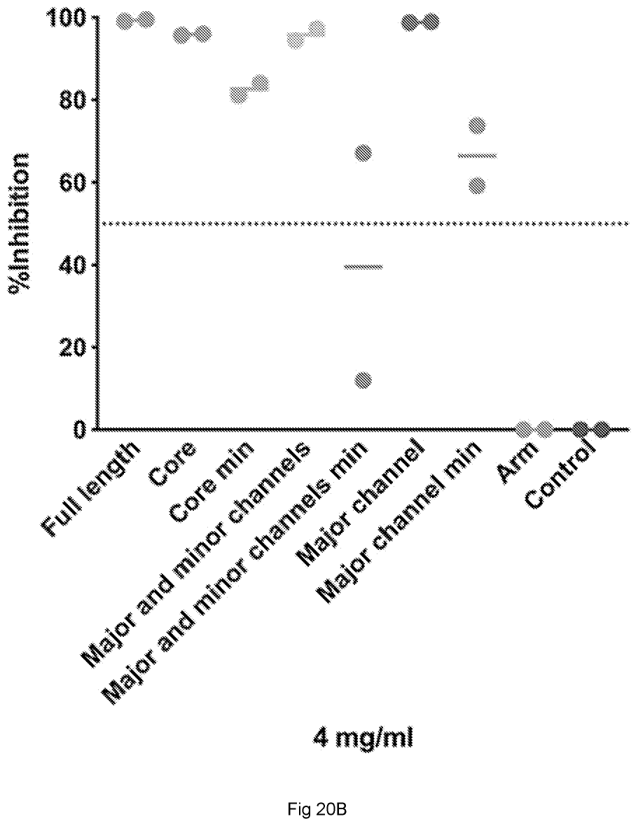

Purified IgG from the serum of immunized rats were pooled and evaluated for the ability to prevent adherence of P. falciparum NF4-infected red blood cells to CSA using the standardized binding inhibition assay (BIA) developed to evaluate placental malaria vaccine candidates. Three designs, as well as the full length ectodomain control, showed robust BIA activity: the core, the major and minor channels, and the major channel. (FIGS. 20A and 20B.) In contrast the other designs, particularly the Arm, showed no activity in this assay. This is significant as a large fraction of the human antibody response is directed to domains in the Arm, and our studies indicate these antibodies are likely non-functional.

### EXAMPLE 8

This Example demonstrates that disclosed immunogen polypeptides showing activity upon immunization with CFA/IFA demonstrate strong activity with AddaS03.

AddaS03 (Invivogen, San Diego, CA) is the research grade equivalent of the clinical AS03 adjuvant approved for human use in multiple successful vaccines. The AddaS03 data presented below are particularly significant as they indicate our findings will be readily translated to human patients.

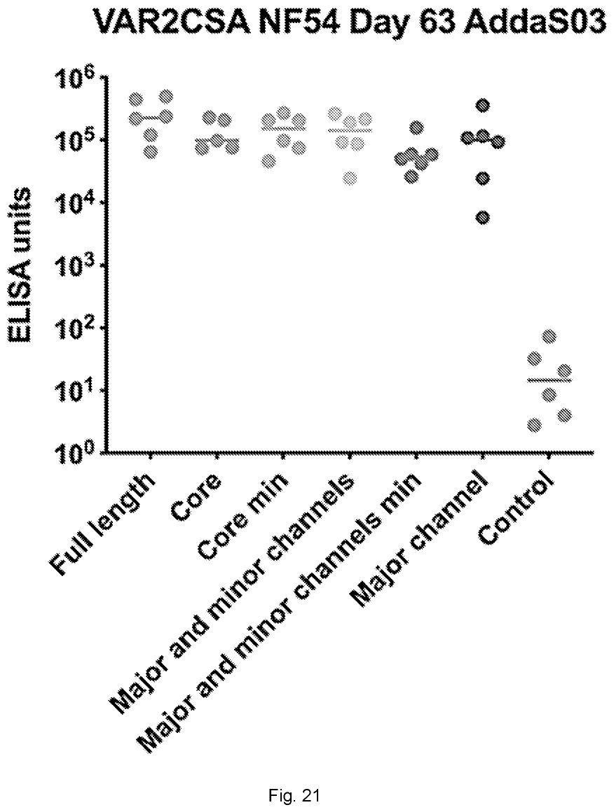

Selected immunogen polypeptides that demonstrated inhibitory activity with CFA/IFA were adjuvanted in AddaS03 and evaluated by immunization of rats. Vaccines were administered on days 0, 21 and 42, and samples were collected on days 14, 35 and 63. Serology studies indicated that all immunogens raise antibody titers greater than adjuvant control, and equal to or greater than the full-length immunogen control after three vaccinations. (FIG. 21.)

### EXAMPLE 9

This Example demonstrates that the core, major and minor channels, and major channel polypeptides adjuvanted in AddaS03 show strong homologous inhibitory activity in assays that are correlated with placental sequestration of parasites.

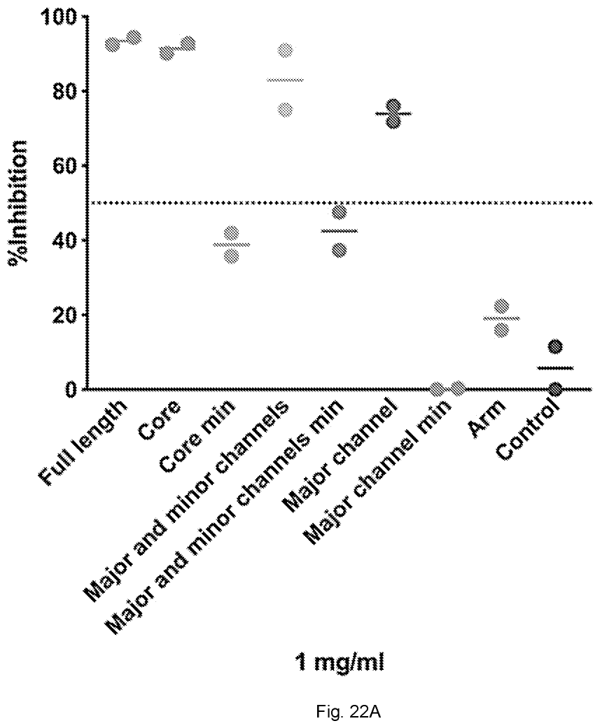

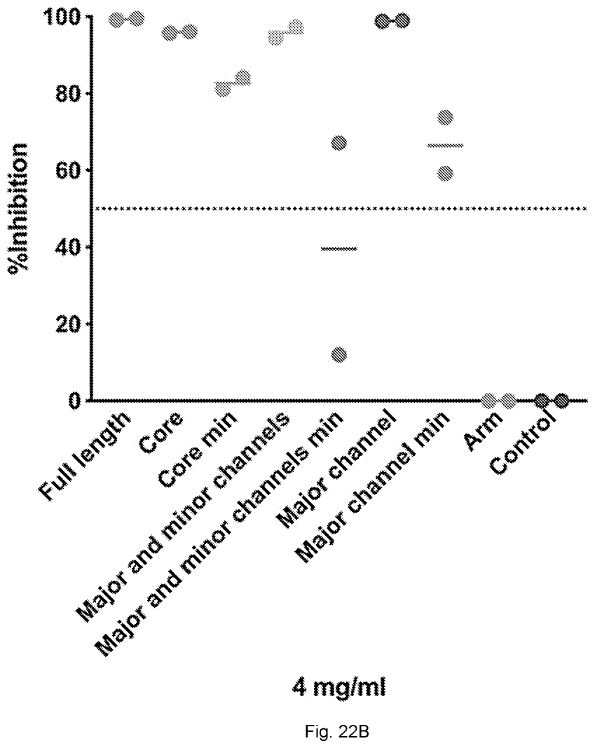

Purified IgG from the serum of immunized rats were pooled and evaluated for the ability to prevent adherence of P. falciparum NF4-infected red blood cells to CSA using the standardized binding inhibition assay (BIA) developed to evaluate placental malaria vaccine candidates. All three designs, as well as the full length ectodomain control, showed robust BIA activity. (FIG. 22A and 22B.)

### EXAMPLE 10

This Example demonstrates the examination of vaccination parameters

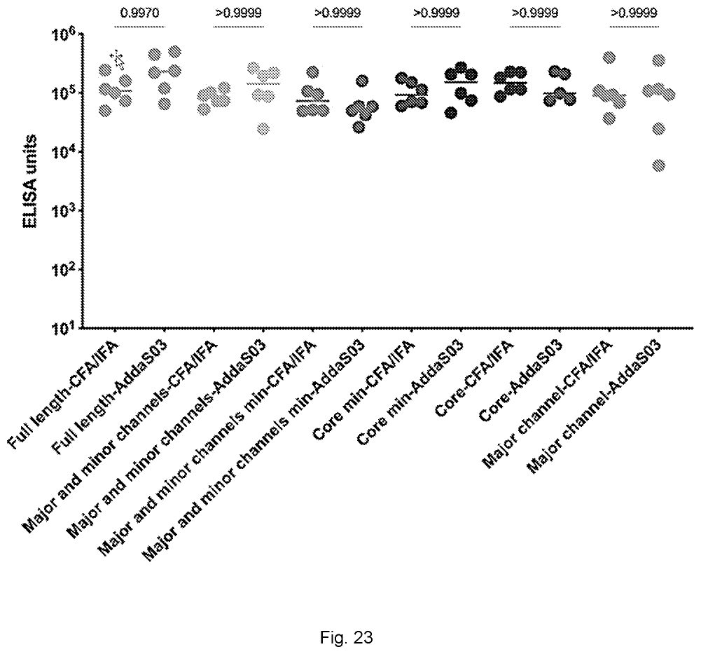

Immunogen polypeptides were formulated using either CFA/IFA or AddaS03. Differences in antibody titers elicited by each formulation are not statistically significant. (FIG. 23.) This indicates that AddaS03 may be a suitable adjuvant.

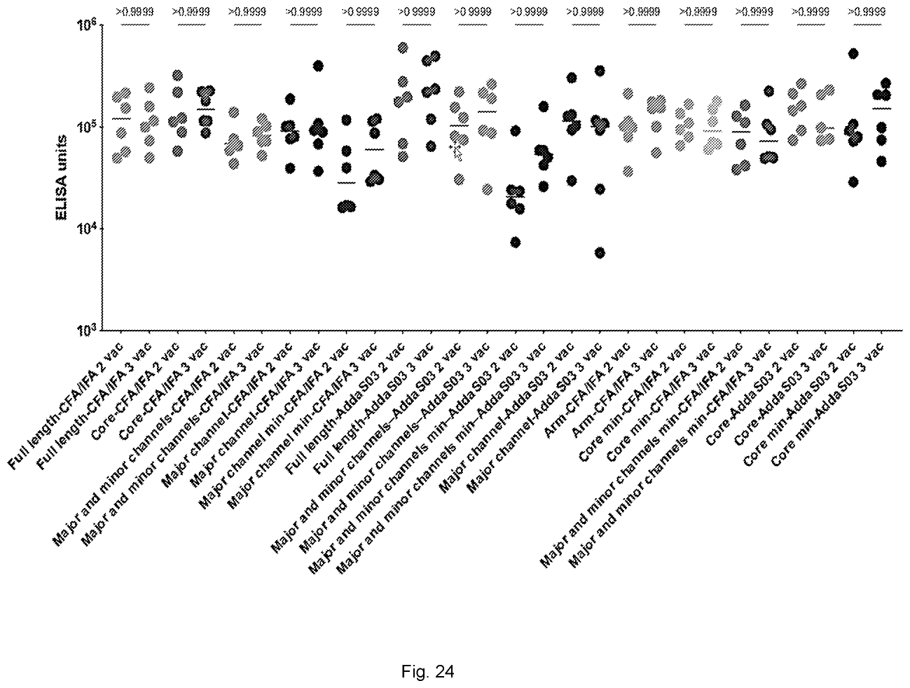

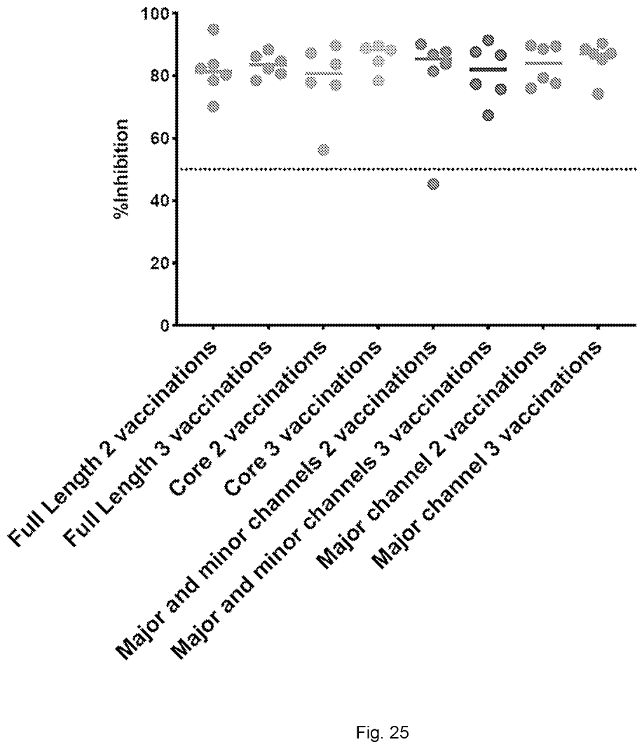

It was also determined that there are no statistical differences in the antibody titers between the second and third vaccination doses. (FIG. 24.) These data indicate a two vaccination regimen may be suitable. Further, immunogen polypeptides formulated with AddaS03 induce strong homologous inhibitory antibodies with either two or three vaccinations. (FIG. 25.)

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An immunogen polypeptide comprising:
a) all or a portion of the major CSA binding channels of VAR2CSA;
b) all or a portion of the minor CSA binding channels of VAR2CSA;
c) all or a portion of the arm segment of VAR2CSA; or
d) combinations thereof.

2. The immunogen polypeptide of claim 1, wherein the immunogen polypeptide comprises all or a portion of each of the NTS, DBL1X, DBL2X and DBL4ε regions of VAR2CSA.

3. The immunogen polypeptide of claim 1, wherein the VAR2CSA fragment comprises all or a portion of ID1.

4. The immunogen polypeptide of claim 1, wherein the immunogen polypeptide comprises all or a portion of each of the ID2a and ID2b regions of VAR2CSA.

5. The immunogen polypeptide of claim 1, wherein the immunogen polypeptide comprises all or a portion of each of the DBL3X region of VAR2CSA.

6. The immunogen polypeptide of claim 1, wherein the immunogen polypeptide comprises all or a portion of the ID3 region of VAR2CSA.

7. The immunogen polypeptide of claim 1, wherein the immunogen polypeptide comprises one or more conserved CSA binding channel residues.

8. The immunogen polypeptide of claim 7, wherein the conserved CSA binding channel residues are located in the major CSA binding channels of VAR2CSA.

9. The immunogen polypeptide of claim 1, wherein the immunogen polypeptide comprises one or more amino acids corresponding to amino acids 44-46, 47-49, 556-558, 560- 563, 575-577, 821-823, 827-836, 911-913, 951-953, 967-971, 1784-1786, 1879-1881, or 1888-1890 of SEQ ID NO: 1.

10. The immunogen polypeptide of claim 9, wherein the immunogen polypeptide comprises one or more amino acids corresponding to amino acids 45, 48, 557, 561, 562, 576, 822, 828, 829, 832, 835, 912, 952, 968, 970, 1785, 1880, 1889 of SEQ ID NO: 1.

11. The immunogen polypeptide of claim 1, wherein the immunogen polypeptide has at least 80% identity with any one of SEQ ID NOs: 3-54, and a pharmaceutically acceptable carrier.

12. The immunogen polypeptide of claim 11, wherein the immunogen polypeptide comprises any one of SEQ ID Nos: 3-54.

13. The immunogen polypeptide of claim 1, wherein the immunogen polypeptide is isolated or purified.

14. A pharmaceutical composition comprising one or more immunogen polypeptides according to claim 1, and a pharmaceutically acceptable carrier.

15. The pharmaceutical composition of claim 14, wherein the composition comprises multimers of the one or more immunogen polypeptides.

16. The pharmaceutical composition of claim 14, wherein the one or more immunogen polypeptides are in nanoparticle form.

17. The pharmaceutical composition of claim 14, further comprising one or more additional therapeutic agents.

18. The pharmaceutical composition of claim 17, wherein the one or more additional therapeutic agents are coupled to the one or more immunogen polypeptides.

19. The pharmaceutical composition of claim 17, wherein the one or more additional therapeutic agents comprise an anti-cancer agent.

20. The pharmaceutical composition of claim 14, wherein the composition is suitable for parenteral administration to a subject

21. A nucleic acid encoding one or more immunogen polypeptides according to claim 1, wherein the nucleic acid is isolated or purified.

22. The nucleic acid of claim 21, wherein the immunogen polypeptide has at least 80% identity with any one of SEQ ID NOs: 3-54.

23. The nucleic acid of claim 22, wherein the immunogen polypeptide comprises any one of SEQ ID NOs: 3-54.

24. A vector comprising the nucleic acid of claim 21.

25. A pharmaceutical composition comprising one or more nucleic acids according to claim 21, and a pharmaceutically acceptable carrier.

26. The pharmaceutical composition of claim 25, wherein the composition is suitable for parenteral administration to a subject.

27. A method for vaccinating a subject against malaria, the method comprising
administering the composition of claim 14 to the subject.

28. The method of claim 27, wherein the malaria is placental malaria.

29. The mehtod of claim 27, wherein the subject is a mammal.

30. The method of claim 29, wherein the mammal is a human.

31. The method of claim 27, wherein the administration results in an immune response in the subject, and wherein the immune response in the subject is sufficient to reduce risk of malaria infection.

33. The method use of claim 32, wherein the immune response in the subject is strain transcending.

34-42. (canceled)

43. A method of producing an immunogen polypeptide of claim 1 in a cell in a subject in need thereof, comprising administering to the subject a pharmaceutical composition comprising a modified messenger RNA (mmRNA) such that the mmRNA is introduced into the cell, wherein the mmRNA comprises a translatable region encoding the immunogen polypeptide, and wherein the immunogen polypeptide is produced in the cell.

44. The immunogen polypeptide of claim 1, wherein the immunogen polypeptide comprises all or a portion of each of the NTS, DBL1X, ID1, DBL2X DBL4ε, and ID3 regions of VAR2CSA.
